(19) United States
(12) Patent Application Publication
(10) Pub. No.: US 2024/0075049 A1
(43) Pub. Date: Mar. 7, 2024

(54) DRUGS, THERAPEUTIC COMBINATIONS AND METHODS FOR PREVENTING VIRAL AND MICROBIAL INFECTIONS AND THEIR SEQUELAE

(71) Applicant: TOPELIA AUST LTD (ACN 652 771 670), Five Dock (AU)

(72) Inventor: Thomas Julius BORODY, Five Dock (AU)

(21) Appl. No.: 18/268,589

(22) PCT Filed: Dec. 20, 2021
(86) PCT No.: PCT/AU2021/051526

§ 371 Date: Jun. 20, 2023

Related U.S. Application Data
(60) Provisional application No. 63/128,152, filed on Dec. 20, 2020.
(60) Provisional application No. 63/178,671, filed on Apr. 23, 2021.
(60) Provisional application No. 63/215,041, filed on Jun. 25, 2021.
(60) Provisional application No. 63/223,432, filed on Jul. 19, 2021.
(60) Provisional application No. 63/241,498, filed on Sep. 7, 2021.
(60) Provisional application No. 63/257,192, filed on Oct. 19, 2021.

(51) Int. Cl. A61K 31/7048; A61K 31/07; A61K 31/13; A61K 31/145; A61K 31/167; A61K 31/216; A61K 31/277; A61K 31/352; A61K 31/4025; A61K 31/4166; A61K 31/427; A61K 31/4402; A61K 31/4965; A61K 31/58; A61K 31/593; A61K 31/65; A61K 31/7052; A61K 31/7056; A61K 31/7068; A61P 31/14

(52) U.S. Cl. CPC A61K 31/7048; A61K 31/07; A61K 31/13; A61K 31/145; A61K 31/167; A61K 31/216; A61K 31/277; A61K 31/352; A61K 31/4025; A61K 31/4166; A61K 31/427; A61K 31/4402; A61K 31/4965; A61K 31/58; A61K 31/593; A61K 31/65; A61K 31/7052; A61K 31/7056; A61K 31/7068; A61P 31/14

(57) ABSTRACT

In alternative embodiments, provided are drugs, therapeutic combinations and methods for preventing or ameliorating, or decreasing the chances of having any adverse effects from, decreasing the severity of adverse effects from, a viral infection such as a coronavirus infection or a microbial infection including a protozoan, helminthiasis, insect and/or parasitic infection such as: malaria that can be caused by a parasite of the genus Plasmodium; filariasis, leprosy or streptocerciasis that can be caused by a parasite of the superfamily Filarioidea; leprosy that can be caused by a parasite of the genus Mycobacterium; river blindness or onchocerciasis that can be caused by parasitic worms such as parasites of the genus Onchocerca; hookworm or roundworm infections that can be caused by parasites of the genus Ancylostoma or Necator; trichuriasis or whipworm infection that can be caused by a parasite of the genus Trichuris; roundworm or an Ascaris infections; mite-carried infections such as scabies; infections such as typhus caused by lice or parasites of the order Phthiraptera; enterobiasis that can be caused by pinworm or parasites of the genus Enterobius; pulicosis or infections cause by fleas or insects of the order Siphonaptera or of the genus Pulex, and other infections and infestations.

### FIELD

This invention generally relates virology, microbiology and infectious diseases. In alternative embodiments, provided are drugs, therapeutic combinations and methods for preventing, or decreasing the chances of having any adverse effects from, decreasing the severity of adverse effects from, or treating or ameliorating a viral infection such as a coronavirus infection (such as COVID-19, or any of its variants, such as delta or omicron variants) or a microbial infection including a protozoan, helminthiasis, insect and/or parasitic infection such as: malaria that can be caused by a parasite of the genus Plasmodium (such as P. vivax, P. falciparum, P. malariae, P. ovale, or P. knowlesi); dengue fever; filariasis, leprosy or streptocerciasis that can be caused by a parasite of the superfamily Filarioidea (such as Brugia malayi, Brugia timori, Wuchereria bancrofti, Loa boa, Mansonella streptocerca, Mansonella ozzardi, or Mansonella perstans); leprosy that can be caused by a parasite of the genus Mycobacterium (such as M. leprae or M. lepromatosis); river blindness or onchocerciasis that can be caused by parasitic worms such as parasites of the genus Onchocerca (such as O. volvulus); hookworm or roundworm infections that can be caused by parasites of the genus Ancylostoma (such as A. duodenale or A. ceylanicum) or Necator (such as N. americanus); trichuriasis or whipworm infection that can be caused by a parasite of the genus Trichuris (such as T. trichiura); roundworm or an Ascaris infection that can be caused by Ascaris lumbricoides; mite-carried infections such as scabies that can be caused by the parasite of the genus Sarcoptes (such as S. scabiei); infections such as typhus caused by lice or parasites of the order Phthiraptera (such as Pediculus humanus capitis); enterobiasis that can be caused by pinworm or parasites of the genus Enterobius (such as E. vermicularis); pulicosis or infections cause by fleas or insects of the order Siphonaptera or of the genus Pulex (such as P. irritans), and other infections and infestations.

### BACKGROUND

Coronavirus infections have previously caused SARS (Severe Acute Respiratory Syndrome) and MERS (Middle East Respiratory Syndrome) and are particularly difficult to treat with anti-viral agents, and single drug regimens have not been found to be effective against the current coronavirus infection called COVID-19. No known anti-infective agents used singly or alone are able to prevent a coronavirus infection.

### SUMMARY OF INVENTION

In alternative embodiments, provided are methods for preventing, or substantially preventing, or decreasing the chances of having any adverse effects from, decreasing the severity of adverse effects from, or treating or ameliorating a viral infection or a microbial infection, or a protozoan, helminthiasis, insect and/or parasitic infection, in an individual in need thereof, comprising administering to the individual in need thereof:
(a) (i) a loading dosage comprising an avermectin class drug (optionally ivermectin) in a dosage of:
   (1) about 300 μg/kg to 30 mg/kg (or 30 mg per 2.2 pounds (lb)) or about 18 mg to 1800 mg in a 60 kg (about 132 lb), or a dosage of 50 μg/kg, 75 μg/kg or 100 μg/kg, or a loading dose of an avermectin class drug (optionally ivermectin) of between about 300 μg/kg to between 30 mg/kg to 60 mg/kg or between about 18 mg to about 1200 mg or 1600 mg to 1800 mg in a 60 kg (about 132 lb) person, or between about 300 μg (mcg) to about 40 to 70 mg/kg, or a dosage of 60 to 120 mg to about 1600 to 1800 mg for an adult; or, (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; and
   (ii) after administration of the loading dosage of (i), administering a maintenance dosage of ivermectin of between about 20 mcg/kg (μ/kg) to 5000 mcg/kg (μ/kg) or between about 200 to 2000 mcg/kg (μ/kg) per dose, where 200 mcg/kg is equivalent to a 12 mg dosage in a 60 kg adult, and 2000 mcg/kg is equivalent to 120 mg per dose, or at about 50 μg/kg, 75 μg/kg or 100 μg/kg;
(b) a drug, a formulation or a therapeutic combination of drugs comprising an avermectin class drug (optionally ivermectin) at a dosage of:
   (i) about 300 μg/kg to 30 mg/kg (or 30 mg per 2.2 pounds (lb)) or about 18 mg to 1800 mg in a 60 kg (about 132 lb), or a dosage of 50 μg/kg, 75 μg/kg or 100 μg/kg, or at a loading dose of ivermectin of between about 30 μg/kg to 60 mg/kg or between about 18 mg to about 1200 mg or 1600 mg to 1800 mg in a 60 kg (about 132 lb) person, or between about 40 to 70 mg/kg, or a dosage of 60 to 120 mg to about 1600 to 1800 mg, or is dosage at 50 μg/kg, 75 μg/kg or 100 μg/kg, for an adult, or
   (ii) between about 18 to 50 mg, or about 18 mg, 24 mg, 30 mg, 36 mg or 40 mg, or between about 50 mg to 100 mg, or 60 to 120 mg to about 1600 to 1800 mg for an adult;
(c) a synthetic nucleoside analog or derivative, or N4-hydroxycytidine, or a prodrug of N4-hydroxycytidine, optionally molnuvpiravir (Merck), or favipiravir (also known as T-705 or AVIGAN™), or favilavir, Toyama Chemical, Fujifilm, Japan, or FABIFLU™ (Glenmark Pharmaceuticals),
wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days;
(d) an anti-androgen drug, and optionally the anti-androgen drug is bicalutamide, optionally CASODEX™, or dutasteride (or AVODART™),
   and optionally the anti-androgen drug is a nonsteroidal anti-androgen (NSAA) or an androgen receptor (AR) antagonist, and optionally the NSAA or AR antagonist comprises proxalutamide (or its developmental name GT-0918) (Suzhou Kintor Pharmaceuticals, Inc., a subsidiary of Kintor Pharmaceutical Limited), or flutamide (or niftolide, or EULEXIN™), or bicalutamide (or CASODEX™) or enzalutamide (or XTANDI™),
   and optionally the anti-androgen drug comprises a 5α-reductase inhibitor, and optionally the 5α-reductase inhibitor comprises finasteride (or PROSCAR™, PROPECIA™, or FINIDE™),
   and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
   and optionally the anti-androgen drug, or NSAA, or bicalutamide, proxalutamide, flutamide or niftolide, bicalutamide, enzalutamide or dutasteride, is administered at dosages of about 50 to 100 mg optionally administered once, twice (BID), three times (TID) or four times a day, or is administered at dosages of about 50 to 100 mg per day,
   and optionally the anti-androgen drug, or NSAA, or bicalutamide, proxalutamide, flutamide or niftolide, bicalutamide, enzalutamide or dutasteride, is administered with an avermectin class drug, or ivermectin, optionally also administered with hydroxychloroquine, zinc, zinc salt or zinc chelate and/or a vitamin (optionally vitamin D (optionally vitamin D2, or ergocalciferol, or Vitamin D3 or cholecalciferol, optionally administered at about 1000 to 4000 ugm/day), or vitamin C, B or A),
   and optionally the anti-androgen drug, or NSAA, or bicalutamide, proxalutamide, flutamide or niftolide, bicalutamide, enzalutamide or dutasteride, is administered with colchicine (or COLCRYS™, MITIGARE™), and optionally also zinc (such as a zinc salt or zinc chelate) and/or a vitamin (optionally vitamin D (optionally vitamin D2, or ergocalciferol, or Vitamin D3 or cholecalciferol, optionally administered at about 1000 to 4000 ugm/day), or vitamin C, B or A),
   and optionally the anti-androgen drug, or NSAA, or bicalutamide, proxalutamide, flutamide or niftolide, bicalutamide, enzalutamide or dutasteride, is administered with an antibiotic (optionally azithromycin or doxycycline), and optionally also zinc (such as a zinc salt or zinc chelate) and/or a vitamin (optionally vitamin D (optionally vitamin D2, or ergocalciferol, or Vitamin D3 or cholecalciferol, optionally administered at about 1000 to 4000 ugm/day), or vitamin C, B or A), and optionally also with hydroxychloroquine;
(e) an anti-malarial drug, wherein optionally the anti-malarial drug comprises mefloquine (or LARIAM™, MEPHAQUIN™, or MEFLIAM™), wherein optionally the mefloquine is formulated for oral administration, optionally in tablet or capsule form, optionally as 200 mg, 250 mg or 300 mg tablets;
(f) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™ or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof,
(g) an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
and optionally the DDS system comprises: a polyether ester urethane comprising 65% D, L-lactide, 19% polyethylene glycol, and 16% glycolide interlinked with an aliphatic di-isocyanate, or comprises VISCOPRENE™,
and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously,
(h) a nicotinic antagonist, a dopamine agonist or a noncompetitive N-Methyl-D-aspartate (NMDA) antagonist, optionally amantadine, or GOCOVRI™, or SYIVIADINE™, or SYMMETREL™, optionally dosaged at between about 100 to 200 mg per dose, optionally formulated as tablets or capsules,
(i) a mitochondrial sensitizer, optionally proguanil or chlorguanide (or PALUDRINE™), or a malarial cytochrome bc1 complex inhibitor, optionally atovaquone (or MEPRON™), or a combination of proguanil and atovaquone (or MALARONE™),
and optionally the proguanil, atovaquone or the combination of proguanil and atovaquone are formulated for oral administration, optionally as tablets, optionally the unit dosage of atovaquone is 250 mg, 300 mg, 350 mg, 400 mg, 500 mg or 1 gram, and the unit dosage of proguanil is 100 mg, 250 mg, 300 mg, 350 mg or 400 mg; and/or
(j) a drug combination or therapeutic regimen comprising any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i).

In alternative embodiments of methods as provided herein:
the avermectin class drug comprises: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;
the drug combination is administered to prevent or substantially prevent, and/or to treat and/or ameliorate, to decrease the symptoms of:
   a coronavirus infection, optionally a COVID-19 infection;
   malaria that can be caused by a parasite of the genus Plasmodium (optionally P. vivax, P. falciparum, P. malariae, P. ovale, or P. knowlesi);
   dengue fever or dengue shock syndrome that can be caused by a virus of the Flaviviridae family or a dengue virus;
   hepatitis or hepatocellular carcinoma associated with viral hepatitis that can be caused by a virus of the Flaviviridae family or a virus of the genus Hepacivirus or Hepacivirus C virus or hepatitis C;
   filariasis, leprosy or streptocerciasis that can be caused by a parasite of the superfamily Filarioidea (optionally Brugia malayi, Brugia timori, Wuchereria bancrofti, Loa loa, Mansonella streptocerca, Mansonella ozzardi, or Mansonella perstans);
   leprosy that can be caused by a parasite of the genus Mycobacterium (optionally M. leprae or M. lepromatosis);
   river blindness or onchocerciasis that can be caused by parasitic worms such as parasites of the genus Onchocerca (optionally O. volvulus);
   hookworm or roundworm infections that can be caused by parasites of the genus Ancylostoma (optionally A. duodenale or A. ceylanicum) or Necator (optionally N. americanus);
   trichuriasis or whipworm infection that can be caused by a parasite of the genus Trichuris (optionally T. trichiura); roundworm or an Ascaris infection that can be caused by Ascaris lumbricoides;
   mite-carried infections such as scabies that can be caused by the parasite of the genus Sarcoptes (optionally S. scabiei);
   infections such as typhus caused by lice or parasites of the order Phthiraptera (optionally Pediculus humanus capitis);
   enterobiasis that can be caused by pinworm or parasites of the genus Enterobius (optionally E. vermicularis); and/or
   pulicosis or infections cause by fleas or insects of the order Siphonaptera or of the genus Pulex (optionally P. irritans);
the loading dose of the avermectin class drug (optionally ivermectin) is between about 15 to 150 mg/kg, or is about 18, 24, 30, 35, 40, 35, 50, 55, 60, 65, 70, 75, 80, 85, 90, 95, 100, 110 or 120 or more mg/kg;
the maintenance dosage of (b) is administered 1, 2, 3, 4, 5, 6, 7, 8, 9, 10, 11, 12, 13 or 14 days, or every 3 weeks or every month or every two months or longer, after the first loading dosage;
the maintenance dosage of (b) is administered every 1, 2, 3, 4, 5, 6, 7, 8, 9, 10, 11, 12, 13 or 14 days, every 3 weeks, or monthly, over the 4 to 8 weeks, 6 to 10 weeks, 8 to 12 weeks, 10 to 20 weeks, 15 to 30 weeks or 20 to 52 weeks, or more, after the initial or loading dose is given;
an antibiotic or anti-viral is administered with the loading dosage of the avermectin class drug (optionally ivermectin); zinc or a zinc salt or zinc chelate is administered with the loading dosage of the avermectin class drug (optionally ivermectin); or zinc, zinc salt or zinc chelate and an antibiotic is administered with the loading dosage of the avermectin class drug (optionally ivermectin), and optionally the antibiotic comprises doxycycline, azithromycin or hydroxychloroquine (HCQ),
and optionally a drug combination, optionally formulated as one formulation (for example, as a tablet capsule) comprises: ivermectin, doxycycline and zinc chelate, or comprises: ivermectin 12 mg, doxycycline 100 mg and zinc chelate 25 mg;
an antibiotic or anti-viral is administered with the maintenance dose of the avermectin class drug (optionally ivermectin); zinc or a zinc salt is administered with the maintenance dosage of the avermectin class drug (optionally ivermectin); or zinc or a zinc salt and an antibiotic is administered with the maintenance dosage of the avermectin class drug (optionally ivermectin), an optionally the antibiotic comprises doxycycline or azithromycin; and/or
an additional drug is or drugs are administered with the loading dose and/or the maintenance dose, of the avermectin class drug (optionally ivermectin), or before the loading dose and/or the maintenance dose, or any time between administration of the loading dose and the maintenance dose,
and optionally the additional drug comprises or drugs comprise one or any combination of:
or any one or more of the following is administered with the drug combination or therapeutic regimen of any combination of (a) to (i) as described above, or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i), as described above;
   an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally meplazumab), a cyclooxygenase (COX) (optionally, COX2) inhibitor, a glucocorticoid (optionally a synthetic glucocorticoid, hydrocortisone, dexamethasone (or DEXTENZA™, OZURDEX™, or NEOFORDEX™) or cortisol, or CORTEF™), plitidepsin or dehydrodidemnin B, or APLIDIN™, or a nonsteroidal anti-inflammatory drug (NSAID), wherein optionally the NSAID comprises indomethacin (or indomethacin) or INDOCID™ or INDOCIN™, or naproxen, or NAPROSYN™ or ALEVE™, or a cyclooxygenase inhibitor, or a COX-1 or an COX-2 inhibitor, or aspirin, or ibuprofen or ADVIL™ MOTRIN™ or NUROFEN™, or celecoxib or CELEBREX™, or parecoxib or DYNASTAT™, or etoricoxib or ARCOXIA™
   and optionally the anti-inflammatory therapy or anti-inflammatory therapy drug is also administered or formulated with an antibiotic (optionally azithromycin or doxycycline), ivermectin, hydroxychloroquine (optionally, PLAQUENIL™) and/or zinc or any zinc salt (optionally zinc sulfate, optionally at (50 mg daily),
   a thiazolide class drug, optionally nitazoxanide (or ALINIA™, NIZONIDE™) or tizoxanide (or 2-Hydroxy-N-(5-nitro-2-thiazolyl)benzamide);
   molnupiravir, optionally co-administered with and/or formulated with an avermectin class drug (optionally ivermectin), an antibiotic (optionally doxycycline or azithromycin) and/or zinc, or co-administered with and/or formulated with ivermectin, hydroxychloroquine, an antibiotic (optionally doxycycline or azithromycin) and/or zinc;
   a mucolytic therapy or drug, optionally acetylcysteine, ambroxol, bromhexine (or BISOLVON™), carbocisteine, erdosteine, mecysteine or dornase alfa, or an expectorant, optionally guaifenesin;
   an H2 antagonist, or H2RA, or H2 blockers, or a compound, drug or formulation that decreases stomach acid production or decreases stomach pH, wherein optionally the compound, drug or formulation comprises famotidine (or PEPCID™), ranitidine (or ZANTAC™), nizatidine (or AXID™ or TAZAC™), roxatidine acetate, lafutidine, or cimetidine (or TAGAMET™), and optionally the famotidine is administered at a dosage of between about 10 to 60 mg per day, or between about 20 to 40 mg per day;
   a dendrimer, optionally astodrimer sodium (Starpharma, Melbourne, Australia);
   an antihistamine class drug such as azelastine, or ASTELIN™, OPTIVAR™, ALLERGODIL™, bepotastine (or TALION™, BEPREVE™), brompheniramine, fexofenadine or ALLEGRA™, pheniramine or AVIL™, or chlorpheniramine;
   a selective serotonin reuptake inhibitor (SSRI) class drug, optionally fluvoxamine, or LUVOX™, FAVERIN™, FLUVOXIN™; a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™ or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof,
   clofazimine, or LAMPENE™, optionally dosaged at about 100 mg per day, or between about 50 mg and 150 mg per day, and optionally also including colchicine;
   clofazimine, or LAMPENE™, optionally dosaged at about 100 mg per day, or between about 50 mg and 150 mg per day, and chloroquine (or ARALEN™), chloroquine phosphate, chloroquine diphosphate and/or hydroxychloroquine (HCQ) (optionally, PLAQUENIL™), optionally also comprising zinc (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and zinc (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day); vitamin B12, vitamin B6 (or pyridoxine); vitamin K; vitamin A; vitamin E; and/or, vitamin C (optionally administered at 500 mg bid), and optionally also including administration of zinc (optionally a zinc sulphate, acetate, gluconate or picolinate, or zinc oxide nanoparticles, optionally at a dosage of between about 1 mg to 250 mg, or about 50 mg per day), and optionally also including colchicine,
   a combination of clofazimine (optionally dosaged at about 100 mg or 150 mg per day, or between about 50 mg and 200 mg per day), fluvoxamine, and zinc (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), fluvoxamine and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally further comprising zinc (optionally a zinc sulphate, acetate, gluconate or picolinate, or zinc oxide nanoparticles, optionally at a dosage of between about 1 mg to 250 mg), and optionally also including colchicine;hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);
   chloroquine (or ARALEN™), chloroquine phosphate, chloroquine diphosphate and/or hydroxychloroquine (HCQ) (optionally, PLAQUENIL™);
   a corticosteroid or glucocorticoid class drug such as ciclesonide (or ALVESCO™, OMNARIS™, OMNIAIR™, ZETONNA™ or ALVESCO™), budesonide (optionally RHINOCORT™ or PULMICORT™), prednisolone (or ORAPRED™), methyl-prednisolone, prednisone (or DELTASONE™ or ORASONE™) or hydrocortisone (or CORTEF™), wherein optionally the corticosteroid or glucocorticoid class drug (optionally ciclesonide) is inhaled;
   a selective estrogen receptor modulator (SERM), or toremifene (or FARESTON™), or clomifene or clomiphene (or CLOMID™, SEROPHENE™);
   an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
   and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin or a tetracycline class drug, wherein optionally the tetracycline class drug comprises doxycycline, or DORYX™, DOXYHEXA™, DOXYLIN™); chloroquine (or ARALEN™), chloroquine phosphate, chloroquine diphosphate and/or hydroxychloroquine (optionally, PLAQUENIL™); zinc, zinc salt or zinc chelate; remdesivir (optionally, GS-5734TH, Gilead Sciences); oseltamivir (or TAMIFLU™); and/or, hydrocortisone; or, any combination thereof;
   a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or a tetracycline tetracycline class drug, and optionally the tetracycline class drug comprises doxycycline, or DORYX™ DOXYHEXA™, DOXYLIN™;
   at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid);
   copper, optionally administered or formulated at a dosage of between about 1 to 200 mg per day, wherein optionally the copper is administered or formulated as cupric chloride and administered intravenously formulated at about 0.4 mg/ml;
   selenium, optionally administered as selenious acid formulated at about 65.4 mcg/ml (or μ/ml), and optionally the selenium is administered at a dosage of between about 50 to 100 optionally between about 60 to 100 μgm per day is administered to an adult, and only up to 60 μgm per day for pediatric patients;
   favipiravir (or T-705 or AVIGAN™ or favilavir, or FABIFLU™, Glenmark Pharmaceuticals), optionally at 800 mg bid;
   zinc, a zinc salt or a zinc chelate (optionally comprising a zinc sulphate, acetate, gluconate or picolinate) or zinc oxide nanoparticles, optionally at a dosage of between about 1 mg to 250 mg;
   colchicine, or COLCRYS™, MITIGARE™;
   at least one antibiotic (wherein optionally the antibiotic is doxycycline (optionally, DORYX™, DOXYHEXA™, DOXYLIN™) (optionally formulated or administered at a dosage of between about 25 mg to 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
   at least one anti-viral drug or medication, or anti-microbial drug, or palliative agent or drug, wherein optionally the anti-viral drug or medication, or anti-microbial drug, is or comprises efavirenz (for example, SUSTIVA™), tenofovir (optionally tenofovir alafenamide or tenofovir disoproxil, or VIREAD™), emtricitabine and tenofovir, nevirapine (or the combination efavirenz with emtricitabine and tenofovir, or ATRIPLA™), amprenavir (for example, AGENERASE™), nelfinavir (for example, VIRACEPT™) and/or remdesivir (for example, GS-5734™, Gilead Sciences), a viral RNA-dependent RNA polymerase inhibitor, optionally favipiravir (optionally AVIGAN™) or sofosbuvir (optionally SOVALDI™, SOFORAL™); or, an adenosine analog (optionally galidesivir, optionally BCX4430, IMMUCILLIN-A™),
   and optionally the anti-viral drug or medication is or comprises an anti-retroviral drug or drug combination, and optionally the anti-retroviral drug or drug combination comprises: darunavir and cobicistat (for example, REZOLSTA™ or PREZCOBIX™); atazanavir (or REYATAZ™) and cobicistat (or EVOTAZ™); a nucleoside analog reverse-transcriptase inhibitor (NRTI) (optionally abacavir, or ZIAGEN™), lamivudine and dolutegravir (TRIUMEQ™); tenofovir (or tenofovir disoproxil or tenofovir disoproxil, or VIREAD™, or emtricitabine) and elvitegravir and cobicistat (for example, STRIBILD™); tenofovir (or disoproxil or emtricitabine) and elvitegravir and cobicistat (COMPLERA™ or EVIPLERA™); efavirenz (optionally, SUSTIVA™), emtricitabine and tenofovir (or ATRIPLA™); lamivudine, nevirapine and stavudine (for example, TRIOMUNE™); atazanavir (or REYATAZ™) and cobicistat (for example, EVOTAZ™); lamivudine and raltegravir (for example, DUTREBIS™); lamivudine and dolutegravir (or DOVATO™); doravirine, lamivudine and tenofovir (for example, DELSTRIGO™); or lamivudine, zidovudine and nevirapine (for example, CUOVIR-N™), and optionally the anti-viral drug or drug combination comprises daclatasvir (optionally DAKLINZA™);
   a viral, or a coronavirus or a COVID-19, protease inhibitor, wherein optionally the protease inhibitor comprises: ASC09 (CAS registry no. 1000287-05-7) (Janssen Research and Development, LLC), ritonavir (optionally NORVIR™), or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir, cobicistat or darunavir and cobicistat, or PF-07321332 (also called nirmatrelvir), PF-07304814 or PF-008335231 (Pfizer), or remdesivir (for example, GS-5734™, Gilead Sciences) or remdesivir (for example, GS5734™, Gilead Sciences) or ritonavir (optionally NORVIR™) in combination with PF-07321332, PF-07304814 or PF-008335231 (Pfizer) optionally as an oral formulation,
and optionally the PF-07321332, or nirmatrelvir, or the combination of nirmatrelvir and ritonavir, or PAXLOVID™, are administered on a twice daily regimen, optionally for five to ten days, optionally unit doses of PF-07321332 is 300 mg, or two 150 mg tablets of PF-07321332, with one 100 mg tablet of ritonavir, optionally given twice-daily for five days, or between about 5 to 21 days:

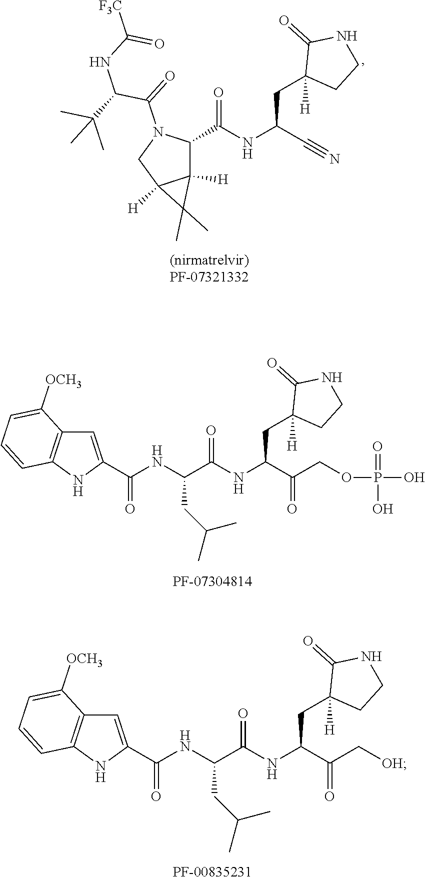 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C ; COc1cccc2[nH]c(C(=O)N[C@@H](CC(C)C)C(=O)N[C@@H](C[C@@H]3CCNC3=O)C(=O)COP(=O)(O)O)cc12 ; COc1cccc2[nH]c(C(=O)N[C@@H](CC(C)C)C(=O)N[C@@H](C[C@@H]3CCNC3=O)C(=O)CO)cc12]

a blood clot inhibiting drug such as aspirin, warfarin (or COUMADIN™) or rivaroxaban (or XARELTO™);
lopinavir, ritonavir (optionally NORVIR™), or the combination lopinavir and ritonavir (or KALETRA™, ALTERA™, ALUVIA™, KALMELTREX or LOPIIVIUNE™), opaganib (or YELIVA™), oseltamivir (or TAMIFLU™), and/or zanamivir (or RELENZA™);
an inhibitor or S-phase kinase-associated protein 2 (SKP2), or dioscin, or niclosamide, or NICLOCIDE™, FENASAL™, or PHENASAL™;
a tyrosine kinase inhibitor (TKi), wherein the TKi comprises: masitinib (or MASIVET™, or KINAVET™); or imatinib (or GLEEVEC™, GLIVEC™); or gefitinib (or IRESSA™), or erlotinib (or TARCEVA™), or dasatinib (or SPRYCEL™, DASANIX™);
ribavirin (optionally NORVIR™) or tribavirin (or COPEGUS™, REBETOL™, or VIRAZOLE™), interferon beta 1b, or a combination of ribavirin and interferon beta, or a combination of lopinavir and ritonavir and interferon-beta-1b;
a nucleoside analog reverse-transcriptase inhibitor (NRTI) (optionally abacavir, or ZIAGEN™) acyclovir or aciclovir (optionally ZOVIRAX™), adefovir (optionally HEPSERA™), amantadine (optionally GOCOVRI™, SYMADINE™, SYMMETREL™), rintatolimod (or AMPLIGEN™), amprenavir (optionally, AGENERASE™), aprepitant (or EMEND™), umifenovir (or ARBIDOL™), atazanavir (or REYATAZ™), tenofovir, a combination of efavirenz and emtricitabine and tenofovir (or ATRIPLA™), balavir, baloxavir marboxil (XOFLUZA™), bepotastine (or TALION™, BEPREVE™), bevirimat, bictegravir, biktarvy, brilacidin, cidofovir, caspofungin, lamivudine and zidovudine (optionally, COMBVIR™), cobicstat (or TYBOST™), colisitin (or polymyxin E, or XYLISTIN™, or COLY-MYCIN M™), cocaine, darunavir (or PREZISTA™), a non-nucleoside reverse transcriptase inhibitor (NNRTI) such as delavirdine (or RESCRIPTOR™), didanosine (or VIDEX™), docosanol (or 1-docosanol, also known as behenyl alcohol), dolutegravir (or TIVICAY™), ecoliever, edoxudine, efavirenz (or SUSTIVA™), elvitegravir (or VITEKTA™), emtricitabine (or EMTRIVA™), enfuvirtide, entecavir (or BARACLUDE™), epirubicin (or ELLENCE™), epoprostenol (or prostacyclin, or FLOLAN™), etravirine (or INTELENCE™), famciclovir (or FAMVIR™), fomivirsen, fosamprenavi, foscarnet (or FOSCAVIR™), fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir (or VIRACEPT™), nevirapine, nexavir, nitazoxanide, ritonavir (or NORVIR™), a nucleoside analogue (optionally brincidofovir (or TEMBEXA™), didanosine (or VIDEX™), favipiravir (also known as T-705 or or AVIGAN™, or favilavir, Toyama Chemical, Fujifilm, Japan), vidarabine, galidesivir (optionally, BCX4430, IMMUCILLIN-A™), remdesivir (optionally, GS-5734TH, Gilead Sciences), cytarabine, gemcitabine, emtricitabine, lamivudine (or EPIVIR™), zalcitabine, entecavir, stavudine (or ZERIT™), telbivudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir (optionally NORVIR™), saquinavir, sofosbuvir, stavudine, telaprevir, tegobuv, tenofovir (optionally tenofovir alafenamide (or VEMLIDY), or tenofovir disoproxil or VIREAD™, or tenofovir with emtricitabine, or DESCOVY™), tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™) or any combination thereof; and/or
or any combination thereof.

In alternative embodiments the following compound (or its isomer, or stereoisomer, or enantiomer, or deuterated version, or bioisostere) is used singly or in various combinations (for example, formulated with, or administered separately) with another drug or drugs, such as an anti-viral drug, for example, with ritonavir; and optionally can be used before, during or after vaccination or administration of a causative agent of infection: (1R,2S,5S)—N-[(1S)-1-cyano-2-[(3S)-2-oxopyrrolidin-3-yl]ethyl]-3-[(2S)-3,3-dimethyl-2-(2,2,2-trifluoroacetamido) butanoyl]-6,6-dimethyl-3-azabicyclo[3.1.0]hexane-2-carboxamide administered orally or by inhalation (or nasally), for example, as liquid, solid, powder, mist or spray, which can target a protease (such as the 3CL protease in COVID-19) and optionally has the following structure and molecular weight:

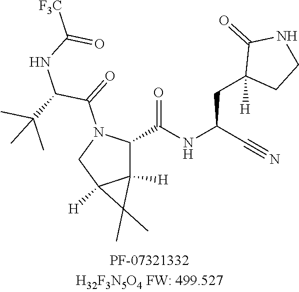 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

This protease inhibitor (PF-07321332, or nirmatrelvir), or the combination of nirmatrelvir and ritonavir, or PAXLOVID™) may be used alone, or optionally before and after the vaccination and/or administration of an attenuated causative agent of infection, optionally administered with ritonavir (or NORVIR′) or lopinavir, or with any of the numerous antiviral agents as provided herein.

In alternative embodiments the following compounds (or their isomers, or stereoisomers, or enantiomer, or bioisostere) can be used singly or in various combinations with drugs, drug combinations or methods as provided herein:

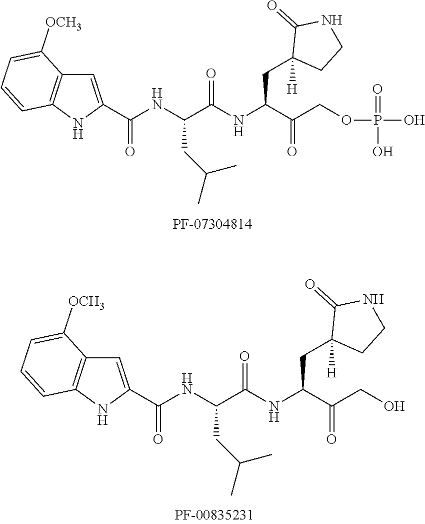 [SMILES: COc1cccc2[nH]c(C(=O)N[C@@H](CC(C)C)C(=O)N[C@@H](C[C@@H]3CCNC3=O)C(=O)COP(=O)(O)O)cc12 ; COc1cccc2[nH]c(C(=O)N[C@@H](CC(C)C)C(=O)N[C@@H](C[C@@H]3CCNC3=O)C(=O)CO)cc12]

These compounds (PF-07304814 and/or PF-00835231) (or its isomer, or stereoisomer, or enantiomer, or deuterated version, or bioisostere) may be used alone before and after the vaccination and/or administration of the attenuated causative agent of infection, optionally administered with ritonavir (or NORVIR™) or lopinavir, or with any of the numerous antiviral agents as provided herein.

In alternative embodiments, the PF-07321332, or nirmatrelvir (or the combination of nirtnatreivir and ritonavir, or PAXLOVID™) and ritonavir (or NORVIR™) or lopinavir combination; or the PF-07304814 and/or PF-00835231 and ritonavir (or NORVIR™) or lopinavir combination; or the KALETRA™, ALTERA™, ALUVIA™, KALMELTREX, LOPIMUNE™ or LOPINAVIR™ and/or zanamivir (or RELENZA™) combination; is administered separately, or together (for example, formulated together) as a tablet, gel, geltab or capsule, as a powder, in a liquid, in a mist or a spray, or as a lozenge. In alternative embodiments, the PF-07321332 (or PAXLOVID™) and ritonavir (or NORVIR™) or lopinavir combination; or the PF-07304814 and/or PF-00835231 and ritonavir (or NORVIR™) or lopinavir combination; or the KALETRA™, ALTERA™, ALUVIA™, KALMELTREX, LOPIMUNE™ or LOPINAVIR™ and/or zanamivir (or RELENZA™) combination; is administered (which in some embodiments the administration prevents the need for hospitalization of an individual in need thereof, or a patient); and in alternative embodiments, the combination the PF-07321332, or nirmatrelvir, or the combination of nirmatrelvir and ritonavir, or PAXLOVID™, and/or a ritonavir (or NORVIR™) or lopinavir combination) is administered before, at the same time as, and/or after an anti-viral (for example, anti-COVID) vaccination.

In alternative embodiments, the PF-07321332, or nirmatrelvir, or the combination of nirmatrelvir and ritonavir, or PAXLOVID™, or a ritonavir (or NORVIR™) and/or lopinavir combination; or the PF-07304814 and/or PF-00835231 and ritonavir (or NORVIR™) or lopinavir combination; or the KALETRA™, ALTERA™, ALUVIA™, KALMELTREX, LOPIMUNE™ or LOPINAVIR™ and/or zanamivir (or RELENZA™) combination; is administered 1, 2, 3, 4, 5, 6, 7, 8, 9, 10, 11, 12, 13 or 14 or more days before, and/or on the day of, a first dose of the at least one of a plurality of dosages of the vaccine is administered, or a dose of the inactivated, attenuated, or a live, viable or infectious causative agent of the infection is administered.

In alternative embodiments, the PF-07321332, or nirmatrelvir, or the combination of nirmatrelvir and ritonavir, Or PAXLOVID™ or the lopinavir combination; or the PF-07304814 and/or PF-00835231 and ritonavir (or NORVIR™) or lopinavir combination; or the KALETRA™, ALTERA™, ALUVIA™, KALMELTREX, LOPIMUNE™ or LOPINAVIR™ and/or zanamivir (or RELENZA™) combination; is administered 1, 2, 3, 4, 5, 6, 7, 8, 9, 10, 11, 12, 13 or 14 or more days after a first dose of the at least one of a plurality of dosages of the vaccine is administered, or a dose of the inactivated, attenuated, or a live, viable or infectious causative agent of the infection is administered.

In alternative embodiments, the PF-07321332, or nirmatrelvir, or the combination of nirmatrelvir and ritonavir, or PAXLOVID™, or the ritonavir (or NORVIR™) or lopinavir combination; or the PF-07304814 and/or PF-00835231 and ritonavir (or NORVIR™) or lopinavir combination; or the KALETRA™, ALTERA™, ALUVIA™, KALMELTREX, LOPIMUNE™ or LOPINAVIR™ and/or zanamivir (or RELENZA™) combination; is administered both before and after a first dose of the at least one of a plurality of dosages of the vaccine is administered, or a dose of the inactivated, attenuated, or the live, viable or infectious causative agent of the infection is administered.

In alternative embodiments, provided are methods for treating, preventing, ameliorating, slowing the progress of, decreasing the severity of a coronavirus infection comprises administering a therapeutic combination of drugs or drug, a pharmaceutical dosage form, a drug delivery device, or a product of manufacture as provided herein to an individual that suffers from long term effects, or chronic effects or symptoms, of the viral infection, also called “long-haulers”, or people who have not fully recovered from COVID-19 weeks or even months after first experiencing symptoms, where some long haulers experience continuous symptoms for weeks or months, while others feel better for weeks, then relapse with old or new symptoms. In alternative embodiments, methods as provided herein are used to prevent the so-called “long-hauler” syndrome, or to treat or prevent continuous symptoms for weeks or months, or to prevent or treat relapsing with old or new symptoms.

In alternative embodiments, provided are products of manufacture comprising a drug or drug combination (or therapeutic drug combination) as used in a method of as provided herein, wherein optionally the product of manufacture comprise or is manufactured or fabricated as a blister pack or package, a clamshell, a tray, a shrink wrap, or equivalent,
and optionally the product of manufacture contains or has fabricated therein a first delivery packet (or, what is indicated or configured on the package, kit or container comprises a blister package, a clamshell, a tray, a shrink wrap and the like to be the first dosage taken by the individual) comprising: dosage of an avermectin class drug (optionally ivermectin); or, a loading dosage of an avermectin class drug (optionally ivermectin),
and optionally the avermectin class drug is dosaged at about 30 mg/kg (or 30 mg per 2.2 pounds (lb)) or about 1200 mg or 1600 mg to about 1800 mg in a 60 kg (about 132 lb) person,
and optionally the loading dosage of the avermectin class drug is between about 30 to 60 mg/kg or is between about 1600 mg to 1800 mg to 3600 mg in a 60 kg (about 132 lb) person,
and optionally the avermectin class drug is dosaged as used in a method of any of the preceding embodiments,
and optionally the product of manufacture contains or has fabricated therein one or more additional delivery packets containing therein an additional drug or drugs, or vitamin or nutritional supplement, optionally an additional drug or drugs, or vitamin or nutritional supplement as used in a method of any of the preceding embodiments.

In alternative embodiments, provided are uses of a drug or drug combination as used in a method or a product of manufacture as provided herein, in the manufacture of a medicament for preventing, or substantially preventing, a viral infection (wherein optionally the viral infection is a coronavirus infection such as COVID-19) or a microbial infection, or a protozoan, helminthiasis, insect and/or parasitic infection, in an individual in need thereof, wherein optionally viral infection is a coronavirus infection, and optionally the coronavirus infection is a COVID-19 infection or strain, Glade, or variant thereof.

In alternative embodiments, provided are drug or drug combination as used in a method or a product of manufacture as provided herein, for use in preventing, or substantially preventing, a viral infection (wherein optionally the viral infection is a coronavirus infection such as COVID-19) or a microbial infection, or a protozoan, helminthiasis, insect and/or parasitic infection, in an individual in need thereof, wherein optionally viral infection is a coronavirus infection, and optionally the coronavirus infection is a COVID-19 infection or strain, Glade, or variant thereof.

In alternative embodiments, provided are kits comprising a drug or drug combination as described herein or as used in any method as provided herein, wherein optionally the kit comprises instructions for practicing a method as provided herein.

In alternative embodiments, drug combinations, methods and kits as provided herein, comprise or comprise use of:
(i) 1R,2S,5S)—N-[(1S)-1-cyano-2-[(3 S)-2-oxopyrrolidin-3-yl]ethyl]-3-[(2S)-3,3-dimethyl-2-(2,2,2-trifluoroacetamido)butanoyl]-6,6-dimethyl-3-azabicyclo[3.1.0]hexane-2-carboxamide, or a compound having the following structure and molecular weight:

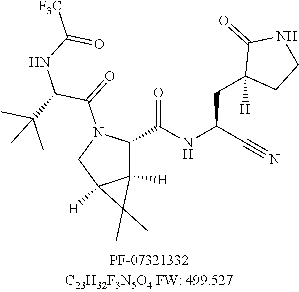 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

or stereoisomer, or enantiomer, or deuterated version thereof, and
(ii) ritonavir,
   which optionally are formulated together, or separately, and optionally are formulated together or separately in or as a liquid (optionally to be administered as a drink or in drops, optionally as nasal drops or in a mist), a tablet, a capsule, a gel, a geltab, a powder, a lozenge, an aerosol or spray.

In alternative embodiments of drugs, drug combinations, methods and kits as provided herein, the anti-viral drug combination is formulated in or as a pharmaceutical dosage form, optionally formulated to be administered orally, intramuscularly, subcutaneously, topically, by use of an enema, intravaginally, or intravenously, or formulated for subcutaneous administration, sublingual administration, inhalation or by aerosol (optionally by inhalation of a liquid, an aerosol, a spray, a mist or a powder), by absorbable patch, by use of an implant, or by use of an enema or a suppository.

In alternative embodiments of drugs, drug combinations, methods and kits as provided herein, the
(a) 1R,2S,5S)—N-[(1S)-1-cyano-2-[(3 S)-2-oxopyrrolidin-3-yl]ethyl]-3-[(2S)-3,3-dimethyl-2-(2,2,2-trifluoroacetamido)butanoyl]-6,6-dimethyl-3-azabicyclo[3.1.0]hexane-2-carboxamide, or a compound having the following structure and molecular weight:

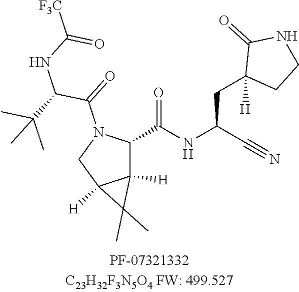 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

or stereoisomer, or enantiomer, or deuterated version thereof, and/or
(b) ritonavir,
   is or are administered:
(a) at a dosage of QD (once a day), bid (twice a day) or tid (three times a day) at a dosage of between about 100 to 600 mg per day or per dosage, or at about 100, 200, 300, 400, 500 or 600 mg per day or per dosage, or
(b) at a dosage of between about 10 mg to 3 gm per dose, or between about 10 mg to 3 gm per day, or 12 mg or 3 mg/kg orally twice daily, or 125 mg orally twice daily or 520 mg/130 mg solution twice per day (optionally administered with efavirenz, fosamprenavir, nelfinavir, or nevirapine), or
(c) is dosed either as a single dose or given one, two, three or four times a day, or
(d) at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days,
(e) for pediatric patients dosage at 16 mg or 4 mg/kg orally twice daily, or
(f) when combined with other drugs a lower dosage, optionally administered at 100 or 200 mg three times a day for between about 5 to 15 days, or for about 7, 8, 9, 10, 11 or 12 days.

In one aspect, forms of the invention may include the following:
1. A method for preventing, or substantially preventing, decreasing the chances of having any adverse effects from, decreasing the severity of adverse effects from, or treating or ameliorating a viral infection or a microbial infection, or a protozoan, helminthiasis, insect and/or parasitic infection, in an individual in need thereof, comprising administering to an individual in need thereof a drug or drug (or therapeutic) combination or composition comprising:
   (a)
      (i) a loading dosage comprising an avermectin class drug (optionally ivermectin) in a dosage of:
      (1) about 300 μg/kg to 30 mg/kg (or 30 mg per 2.2 pounds (lb)) or about 18 mg to 1800 mg in a 60 kg (about 132 lb), or a loading dose of the avermectin class drug (optionally ivermectin) of between about 300 μg/kg to 30 to 60 mg/kg or between about 18 mg to about 1200 mg or 1600 mg to 1800 mg in a 60 kg (about 132 lb) person, or between about 300 μgm (mcg) to 40 mg/kg or 70 mg/kg, or a dosage of between about 120 mg to 280 mg to about 1600 to 1800 mg for an adult; or
      (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; and
      (ii) after administration of the loading dosage of (i), administering a maintenance dosage of ivermectin of between about 20 mcg/kg (μ/kg) to 5000 mcg/kg (μ/kg) or between about 200 to 2000 mcg/kg (μ/kg) per dose, where 200 mcg/kg is equivalent to a 12 mg dosage in a 60 kg adult, and 2000 mcg/kg is equivalent to 120 mg per dose;
   (b) a drug, a formulation or a therapeutic combination of drugs comprising an avermectin class drug (optionally ivermectin) at a dosage of:
      (1) about 300 μg/kg to 30 mg/kg (or 30 mg per 2.2 pounds (lb)) or about 18 mg to 1800 mg in a 60 kg (about 132 lb), or at a loading dose of the avermectin class drug (optionally ivermectin) of between about 300 μg/kg to 30 to 60 mg/kg or between about 18 mg to about 1200 mg or 1600 mg to 1800 mg in a 60 kg (about 132 lb) person, or between about 300 μgm (mcg) to 40 mg/kg or 70 mg/kg, or a dosage of between about 120 mg to 280 mg to about 1600 to 1800 mg for an adult, or
      (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult;
   (c) a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™, Glenmark Pharmaceuticals),
   wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days;
   (d) an anti-androgen drug, and optionally the anti-androgen drug is bicalutamide, optionally CASODEX™, or dutasteride (or AVODART™),
      and optionally the anti-androgen drug is a nonsteroidal anti-androgen (NSAA) or an androgen receptor (AR) antagonist, and optionally the NSAA or AR antagonist comprises proxalutamide (or its developmental name GT-0918) (Suzhou Kintor Pharmaceuticals, Inc., a subsidiary of Kintor Pharmaceutical Limited), or flutamide (or niftolide, or EULEXIN™), or bicalutamide (or CASODEX™) or enzalutamide (or XTANDI™),
      and optionally the anti-androgen drug comprises a 5α-reductase inhibitor, and optionally the 5α-reductase inhibitor comprises finasteride (or PROSCAR™, PROPECIA™ or FINIDE™),
      and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
      and optionally the anti-androgen drug, or NSAA, or bicalutamide, proxalutamide, flutamide or niftolide, bicalutamide, enzalutamide or dutasteride, is administered with an avermectin class drug, or ivermectin, optionally also administered with hydroxychloroquine, zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A),
      and optionally the anti-androgen drug, or NSAA, or bicalutamide, proxalutamide, flutamide or niftolide, bicalutamide, enzalutamide or dutasteride, is administered with colchicine (or COLCRYS™, MITIGARE™), and optionally also zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A),
      and optionally the anti-androgen drug, or NSAA, or bicalutamide, proxalutamide, flutamide or niftolide, bicalutamide, enzalutamide or dutasteride, is administered with an antibiotic (optionally azithromycin or doxycycline), and optionally also zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A), and optionally also with hydroxychloroquine;
   (e) an anti-malarial drug, wherein optionally the anti-malarial drug comprises mefloquine (or LARIAM™, MEPHAQUIN™, or MEFLIAM™), wherein optionally the mefloquine is formulated for oral administration, optionally in tablet or capsule form, optionally as 200 mg, 250 mg or 300 mg tablets;
   (f) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™ or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof,
   (g) an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
   and optionally the DDS system comprises: a polyether ester urethane comprising 65% D, L-lactide, 19% polyethylene glycol, and 16% glycolide interlinked with an aliphatic di-isocyanate, or comprises VISCOPRENE™,
   and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously,
   (h) a nicotinic antagonist, a dopamine agonist or a noncompetitive N-Methyl-D-aspartate (NMDA) antagonist, optionally amantadine, or GOCOVRI™, or SYIVIADINE™, or SYMMETREL™, optionally dosaged at between about 100 to 200 mg per dose, optionally formulated as tablets or capsules, or
   (i) a mitochondrial sensitizer, optionally proguanil or chlorguanide (or PALUDRINE™), or a malarial cytochrome bc1 complex inhibitor, optionally atovaquone (or MEPRON™), or a combination of proguanil and atovaquone (or MALARONE™), and/or
   (j) a drug combination or therapeutic regimen comprising any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i).
2. The method of form 1, wherein the avermectin class drug comprises: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin.
3. The method of form 1 or form 2, wherein the drug or drug combination is administered to prevent or substantially prevent, or to treat or ameliorate, or decrease the severity of symptoms or pathology of:
   a viral infection, optionally a coronavirus, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection,
   a coronavirus infection, optionally a COVID-19 infection, optionally a COVID-19 variant infection, wherein optionally the COVID-19 variant is a delta or an omicron variant, or the coronavirus infection comprises a Middle East respiratory syndrome virus (MERS-CoV) infection;
   malaria that can be caused by a parasite of the genus Plasmodium (optionally P. vivax, P. falciparum, P. malariae, P. ovale, or P. knowlesi);
   dengue fever or dengue shock syndrome that can be caused by a virus of the Flaviviridae family or a dengue virus;
   hepatitis or hepatocellular carcinoma associated with viral hepatitis that can be caused by a virus of the Flaviviridae family or a virus of the genus Hepacivirus or Hepacivirus C virus or hepatitis C;
   filariasis, leprosy or streptocerciasis that can be caused by a parasite of the superfamily Filarioidea (optionally Brugia malayi, Brugia timori, Wuchereria bancrofti, Loa loa, Mansonella streptocerca, Mansonella ozzardi, or Mansonella perstans);
   leprosy that can be caused by a parasite of the genus Mycobacterium (optionally M leprae or M. lepromatosis);
   river blindness or onchocerciasis that can be caused by parasitic worms such as parasites of the genus Onchocerca (optionally O. volvulus);
   hookworm or roundworm infections that can be caused by parasites of the genus Ancylostoma (optionally A. duodenale or A. ceylanicum) or Necator (optionally N. americanus);
   trichuriasis or whipworm infection that can be caused by a parasite of the genus Trichuris (optionally T. trichiura); roundworm or an Ascaris infection that can be caused by Ascaris lumbricoides;
   mite-carried infections such as scabies that can be caused by the parasite of the genus Sarcoptes (optionally S. scabiei);
   infections such as typhus caused by lice or parasites of the order Phthiraptera (optionally Pediculus humanus capitis);
   enterobiasis that can be caused by pinworm or parasites of the genus Enterobius (optionally E. vermicularis); and/or
   pulicosis or infections cause by fleas or insects of the order Siphonaptera or of the genus Pulex (optionally P. irritans).
4. The method of any one of forms 1 to 3, wherein the loading dose of the avermectin class drug (optionally ivermectin) of between is about 15 to 150 mg/kg, or is about 18, 24, 30, 35, 40, 35, 50, 55, 60, 65, 70, 75, 80, 85, 90, 95, 100, 110 or 120 or more mg/kg.
5. The method of any one of the preceding forms, wherein the loading dosage is given once, or periodically, optionally every 2, 3, 4, 5, 6, 7, 8, 9, 10, 11, or 12 or more days.
6. The method of any one of the preceding forms, wherein the maintenance dosage of (a)(ii) is administered 1, 2, 3, 4, 5, 6, 7, 8, 9, 10, 11, 12, 13 or 14 days, or every 3 weeks or every month or every two months or longer after the first loading dosage.
7. The method of any one of the preceding forms, wherein the maintenance dosage of (a)(ii) is administered every 1, 2, 3, 4, 5, 6, 7, 8, 9, 10, 11, 12, 13 or 14 days, every 3 weeks, or monthly, over the 4 to 8 weeks, 6 to 10 weeks, 8 to 12 weeks, 10 to 20 weeks, 15 to 30 weeks or 20 to 52 weeks, or more, after the initial or loading dose is given.
8. The method of any one of the preceding forms, wherein: an antibiotic or anti-viral is administered with the loading dosage of the avermectin class drug (optionally ivermectin); zinc or a zinc salt or zinc chelate is administered with the loading dosage of the avermectin class drug (optionally ivermectin); or zinc or zinc chelate or a zinc salt and an antibiotic is administered with the loading dosage of the avermectin class drug (optionally ivermectin),
and optionally a drug combination, optionally formulated as one formulation (for example, as a tablet capsule) comprises: ivermectin, doxycycline and zinc chelate, or comprises: ivermectin 12 mg, doxycycline 100 mg and zinc chelate 25 mg.
9. The method of form 8, wherein the antibiotic comprises doxycycline, azithromycin or hydroxychloroquine (HCQ).
10. The method of any one of the preceding forms, wherein: an antibiotic or anti-viral is administered with the maintenance dose of the avermectin class drug (optionally ivermectin); zinc or a zinc salt or zinc chelate is administered with the maintenance dosage of the avermectin class drug (optionally ivermectin); or zinc or a zinc salt or zinc chelate and an antibiotic or anti-viral is administered with the maintenance dosage of the avermectin class drug (optionally ivermectin).
11. The method of form 10, wherein the antibiotic comprises doxycycline, azithromycin or hydroxychloroquine (HCQ).
12. The method of any one of the preceding forms, wherein an additional drug is or drugs, or therapy, is or are administered with the loading dose and/or the maintenance dose of the avermectin class drug (optionally ivermectin), or before the loading dose and/or the maintenance dose, or any time between administration of the loading dose and the maintenance dose.
13. The method of any one of the preceding forms, wherein an additional drug or therapy, is or are administered with the drug or drug combination of (a), (b), (c), (d), (e), (f), (g), (h) or (i), or any one or more of the following is administered with the drug combination or therapeutic regimen of any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i) of form 1:
   a thiazolide class drug, optionally nitazoxanide (or Alinia™, Nizonide™) or tizoxanide (or 2-Hydroxy-N-(5-nitro-2-thiazolyl)benzamide);
   molnupiravir, optionally co-administered with and/or formulated with an avermectin class drug (optionally ivermectin), an antibiotic (optionally doxycycline or azithromycin) and/or zinc, zinc salt or zinc chelate, or co-administered with and/or formulated with ivermectin, hydroxychloroquine, an antibiotic (optionally doxycycline or azithromycin) and/or zinc, zinc salt or zinc chelate;
   a mucolytic therapy or drug, optionally acetylcysteine, ambroxol, bromhexine (or BISOLVON™), carbocisteine, erdosteine, mecysteine or dornase alfa, or an expectorant, optionally guaifenesin;
   an H2 antagonist, or H2RA, or H2 blockers, or a compound, drug or formulation that decreases stomach acid production or decreases stomach pH, wherein optionally the compound, drug or formulation comprises famotidine (or PEPCID™), ranitidine (or ZANTAC™), nizatidine (or AXID™ or TAZAC™), roxatidine acetate, lafutidine, or cimetidine (or TAGAMET™), and optionally the famotidine is administered at a dosage of between about 10 to 60 mg per day, or between about 20 to 40 mg per day;
   a dendrimer, optionally astodrimer sodium (Starpharma, Melbourne, Australia);
   an antihistamine class drug such as azelastine, or ASTELIN™, OPTIVAR™, ALLERGODIL™, bepotastine (or TALION™, BEPREVE™) brompheniramine, fexofenadine or ALLEGRA™, pheniramine or AVIL™, or chlorpheniramine;
   a selective serotonin reuptake inhibitor (SSRI) class drug, optionally fluvoxamine, or LUVOX™, FAVERIN™, FLUVOXIN™;
   a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™ or ronifibrate, or simfibrate or CHOLESOLVIN™, or any combination thereof, clofazimine, or LAMPENE™, optionally dosaged at about 100 mg per day, or between about 50 mg and 150 mg per day, and optionally also including colchicine;
   clofazimine, or LAMPENE™, optionally dosaged at about 100 mg per day, or between about 50 mg and 150 mg per day, and chloroquine (or ARALEN™), chloroquine phosphate, chloroquine diphosphate and/or hydroxychloroquine (HCQ) (optionally, PLAQUENIL™), optionally also comprising zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally also including administration of zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine, a combination of clofazimine (optionally dosaged at about 100 mg or 150 mg per day, or between about 50 mg and 200 mg per day), fluvoxamine, and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), fluvoxamine and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally further comprising zinc, zinc salt or zinc chelate (optionally a zinc sulphate, acetate, gluconate or picolinate, or zinc oxide nanoparticles, optionally at a dosage of between about 1 mg to 250 mg), and optionally also including colchicine;
   hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);
   chloroquine (or ARALEN™), chloroquine phosphate, chloroquine diphosphate and/or hydroxychloroquine (HCQ) (optionally, PLAQUENIL™);
   a corticosteroid or glucocorticoid class drug such as such as ciclesonide (or Alvesco™, Omnaris™, Omniair™, Zetonna™ or Alvesco™), budesonide (optionally RHINOCORT™ or PULMICORT™), prednisolone (or ORAPRED™), methyl-prednisolone, prednisone (or DELTASONE™ or ORASONE™) or hydrocortisone (or CORTEF™), or a selective estrogen receptor modulator (SERM), or toremifene (or Fareston™), or clomifene or clomiphene (or CLOMID™, SEROPHENE™), wherein optionally the mode of administration for the corticosteroid or glucocorticoid class drug (optionally ciclesonide) is by inhalation (i.e., they are inhaled);
   a hydrocortisone or cortisol (optionally CORTEF™, SOLUCORTEF™), optionally hydrocortisone sodium succinate or hydrocortisone acetate or dexamethasome (optionally Dextenza™, Ozurdex™, Neofordex™),
      and optionally the corticosteroid or glucocorticoid class drug (for example budesonide or ciclesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,
      and optionally the corticosteroid or glucocorticoid class drug (optionally budesonide or ciclesonide) is formulated as a powder or for administration in an inhaler or by nasal spray, or for rectal administration,
      and optionally the corticosteroid or glucocorticoid class drug (for example, budesonide or ciclesonide) is administered together with or in combination with 10 mg to 80 mg, an antibiotic (optionally azithromycin or a tetracycline class drug,
      wherein optionally the tetracycline class drug comprises doxycycline, or DORYX™, DOXYHEXA™, DOXYLIN™), zinc, zinc salt or zinc chelate and/or a vitamin (optionally vitamin D or calcifediol, D2 (or ergocalciferol), D3 (or cholecalciferol), C, E, B12, B6);
   an anti-androgen drug, and optionally the anti-androgen drug is bicalutamide, optionally CASODEX™, or dutasteride (or AVODART™),
      and optionally the anti-androgen drug is a nonsteroidal anti-androgen (NSAA) or an androgen receptor (AR) antagonist, and optionally the NSAA or AR antagonist comprises proxalutamide (or its developmental name GT-0918) (Suzhou Kintor Pharmaceuticals, Inc., a subsidiary of Kintor Pharmaceutical Limited), or flutamide (or niftolide, or EULEXIN™), or bicalutamide (or CASODEX™) or enzalutamide (or XTANDI™),
      and optionally the anti-androgen drug comprises a 5α-reductase inhibitor, and optionally the 5α-reductase inhibitor comprises finasteride (or PROSCAR™, PROPECIA™, or FINIDE™),
      and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;
   an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2),
      and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin or a tetracycline class drug, wherein optionally the tetracycline class drug comprises doxycycline, or DORYX™, DOXYHEXA™, DOXYLIN™); chloroquine (or ARALEN™), chloroquine phosphate, chloroquine diphosphate and/or hydroxychloroquine (optionally, PLAQUENIL™); zinc, zinc salt or zinc chelate; remdesivir (optionally, GS-5734™, Gilead Sciences); oseltamivir (or TAMIFLU™); and/or, hydrocortisone; or, any combination thereof;
   a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or a tetracycline tetracycline class drug, and optionally the tetracycline class drug comprises doxycycline, or DORYX™ DOXYHEXA™, DOXYLIN™;
   a dendrimer, optionally astodrimer sodium (Starpharma, Melbourne, Australia);
   an antihistamine class drug such as azelastine, or ASTELIN™, OPTIVAR™, ALLERGODIL™, brompheniramine, fexofenadine or ALLEGRA™, pheniramine or AVIL™, or chlorpheniramine;
   a selective serotonin reuptake inhibitor (SSRI) class drug, optionally fluvoxamine, or LUVOX™, FAVERIN™, FLUVOXIN™;
   a nicotinic antagonist, a dopamine agonist or a noncompetitive N-Methyl-d-aspartic acid or N-Methyl-d-aspartate (NMDA) antagonist, wherein optionally the nicotinic antagonist, dopamine agonist or noncompetitive NMDA antagonist is 1-adamantylamine or amantadine, or GOCOVRI™ SYIVIADINE™, SYMMETREL™, optionally administered or dosaged at between about 50 mg to 150 mg, or about 100 mg, or 200 mg, per day for a period of between about 7 and 21 days, or about 14 days, and optionally the nicotinic antagonist, dopamine agonist or noncompetitive NMDA antagonist is also administered or formulated with an antibiotic (optionally azithromycin or doxycycline), ivermectin, hydroxychloroquine (optionally, PLAQUENIL™) and/or zinc, zinc salt or zinc chelate (optionally zinc sulfate, optionally at (50 mg daily), and optionally the amantadine is formulated or administered at 100 mg per day for the first two days of treatment, which optionally can then be elevated to 100 mg twice daily, optionally for the next 10 days;
   an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
      and optionally the DDS system comprises: a polyether ester urethane comprising 65% d,l-lactide, 19% polyethylene glycol, and 16% glycolide interlinked with an aliphatic di-isocyanate, or comprises VISCOPRENE™
      and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously;
   an immunosuppressive drug, wherein optionally the immunosuppressive drug comprises tocilizumab or atlizumab, or Actemra™, or RoActemra™, or a calcineurin inhibitor (CNI), wherein the CNI comprises ciclosporin (or cyclosporine or cyclosporin), or Neoral™, or Sandimmune™, or tacrolimus, or Protopic™, or Prograf™, and optionally the immunosuppressive drug is also administered or formulated with an antibiotic (optionally azithromycin or doxycycline), ivermectin, hydroxychloroquine (optionally, PLAQUENIL™) and/or zinc, zinc salt or zinc chelate (optionally zinc sulfate, optionally at (50 mg daily),
      and optionally the calcineurin inhibitor (CNI), wherein the CNI comprises ciclosporin (or cyclosporine or cyclosporin) is formulated combination of CNI (optionally cyclosporine) at a dose of 3 mg/kg (180 mg daily) together with 12 mg ivermectin once, and optionally also plus zinc 50 mg base and doxycycline 100 mg bid, optionally all for 10 days;
   a protein kinase inhibitor, wherein optionally the protein kinase inhibitor is a p38 mitogen-activated protein kinase inhibitor, or ralimetinib, and optionally the protein kinase inhibitor is also administered or formulated with an antibiotic (optionally azithromycin or doxycycline), ivermectin, hydroxychloroquine (optionally, PLAQUENIL™) and/or zinc or any zinc salt or zinc chelate (optionally zinc sulfate, optionally at (50 mg daily);
   an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally meplazumab), a cyclogenase (COx) (optionally COX2) inhibitor, a glucocorticoid (optionally a synthetic glucocorticoid, hydrocortisone, dexamethasone (or DEXTENZA™, OZURDEX™, or NEOFORDEX™) or cortisol, or CORTEF™), plitidepsin or dehydrodidemnin B, or APLIDIN™, or a nonsteroidal anti-inflammatory drug (NSAID), wherein optionally the NSAID comprises indomethacin (or indomethacin) or INDOCID™ or INDOCIN™, or naproxen, or NAPROSYN™ or ALEVE™, or a cyclooxygenase inhibitor, or a COX-1 or an COX-2 inhibitor, or aspirin, or ibuprofen or ADVIL™ MOTRIN™ or NUROFEN™, or celecoxib or CELEBREX™, or parecoxib or DYNASTAT™, or etoricoxib or ARCOXIA™
      and optionally the anti-inflammatory therapy or anti-inflammatory therapy drug is also administered or formulated with an antibiotic (optionally azithromycin or doxycycline), ivermectin, hydroxychloroquine (optionally, PLAQUENIL™) and/or zinc or any zinc salt or zinc chelate (optionally zinc sulfate, optionally at (50 mg daily),
      and optionally opaganib, or YELIVA™, or opaganib, or YELIVA™ administered or formulated together with an oral and/or inhaled or aerosol chloroquine (or ARALEN™), chloroquine phosphate, chloroquine diphosphate and/or hydroxychloroquine (optionally, PLAQUENIL™),
      and optionally the opaganib or YELIVA™ is formulated or administered at a dosage of QD (once a day), bid (twice a day) or tid (three times a day) at a dosage of between about 100 to 600 mg per day or per dosage, or at about 100, 200, 300, 400, 500 or 600 mg per day or per dosage,
   and optionally the opaganib, or YELIVA™ is also administered or formulated with an antibiotic (optionally azithromycin or doxycycline), ivermectin (optionally at 12 mg ivermectin, optionally administered on days 1, 3, 6 and 8), hydroxychloroquine (optionally, PLAQUENIL™) and/or zinc or any zinc salt or zinc chelate (optionally zinc sulfate, optionally at (50 mg daily);
   a calcium channel blocker, or verapamil (or ISOPTIN™, CALAN™), or a voltage gated potassium (KCNH2) channel or a voltage gated calcium channel (CACNA2D2) blocker, or amiodarone (or CORDARONE™, NEXTERONE™);
   a suramin, or ANTRYPOL™, BAYER 305™, or GERMANIN™;
   a PPAR agonist, optionally fenofibrate, or TRICOR™, FENOBRAT™, FENOGLIDE™, or LIPOFEN™, and optionally the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™ or ronifibrate, or simfibrate or CHOLESOLVIN™, or any combination thereof, or a combination of fenofibrate and simvastatin, or CHOLIB™;
   a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™ Glenmark Pharmaceuticals),
      wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days,
      and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin),
      and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin) with an antibiotic, and optionally the antibiotic comprises azithromycin, minocycline, amoxicillin, niclosamide, nitazoxanide, hydroxychloroquine or doxycycline), and optionally the synthetic nucleoside analog or derivative, avermectin class drug, and antibiotic are administered together or as separate formulations, and optionally are administered every one, two, three, four or five weeks for between about one month and one year or more;
      and optionally molnuvpiravir, ivermectin and hydroxychloroquine are administered together or as separate formulations, and optionally are administered every one, two, three, four or five weeks for between about one month and one year or more;
      and optionally the synthetic nucleoside analog or derivative (optionally N4-hydroxycytidine, or the prodrug of N4-hydroxycytidine, optionally molnuvpiravir or favipiravir), and antibiotic (optionally doxycycline or hydroxychloroquine) is administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D),
      and optionally the synthetic nucleoside analog or derivative, or N4-hydroxycytidine, or the prodrug of N4-hydroxycytidine, optionally molnuvpiravir or favipiravir, is administered with an antibiotic (optionally the antibiotic comprises azithromycin, minocycline, amoxicillin, niclosamide, nitazoxanide, hydroxychloroquine or doxycycline), optionally also administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
         and optionally any of these combinations is administered very 2, 3, 4, 5, 6, 7, 8, 9 or 10 or more days for between about 1 month and one year or more;
   an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
   at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid);
   copper, optionally administered or formulated at a dosage of between about 1 to 200 mg per day, wherein optionally the copper is administered or formulated as cupric chloride and administered intravenously formulated at about 0.4 mg/ml;
   selenium, optionally administered as selenious acid formulated at about 65.4 mcg/ml (or μ/ml), and optionally the selenium is administered at a dosage of between about 50 to 100 optionally between about 60 to 100 μgm per day is administered to an adult, and only up to 60 μgm per day for pediatric patients;
   favipiravir (or T-705, avigan, or favilavir), optionally at 800 mg bid;
   zinc, zinc salt or zinc chelate (optionally a zinc sulphate, acetate, gluconate or picolinate) or zinc oxide nanoparticles, optionally at a dosage of between about 1 mg to 250 mg;
   colchicine, or COLCRYS™, MITIGARE™;
   at least one antibiotic or anti-viral (wherein optionally the antibiotic is doxycycline (optionally, DORYX™, DOXYHEXA™, DOXYLIN™) (optionally formulated or administered at a dosage of between about 25 mg to 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
   at least one anti-viral drug or medication, or anti-microbial drug, or palliative agent or drug, wherein optionally the anti-viral drug or medication, or anti-microbial drug, is or comprises efavirenz (for example, SUSTIVA™), tenofovir (optionally tenofovir alafenamide or tenofovir disoproxil, or VIREAD™), emtricitabine and tenofovir, nevirapine (or the combination efavirenz with emtricitabine and tenofovir, or ATRIPLA™), amprenavir (for example, AGENERASE™), nelfinavir (for example, VIRACEPT™) and/or remdesivir (for example, GS-5734™, Gilead Sciences), a viral RNA-dependent RNA polymerase inhibitor, optionally favipiravir (optionally AVIGAN™) or sofosbuvir (optionally SOVALDI™, SOFORAL™); or, an adenosine analog (optionally galidesivir, optionally BCX4430, INIMUCILLIN-A™),
      and optionally the anti-viral drug or medication is or comprises an anti-retroviral drug or drug combination, and optionally the anti-retroviral drug or drug combination comprises: darunavir and cobicistat (for example, REZOLSTA™ or PREZCOBIX™); atazanavir (or REYATAZ™) and cobicistat (or EVOTAZ™); abacavir, lamivudine and dolutegravir (TRIUMEQ™); tenofovir (or tenofovir disoproxil or tenofovir disoproxil, or VIREAD™, or emtricitabine) and elvitegravir and cobicistat (for example, STRIBILD™); tenofovir (or disoproxil or emtricitabine) and elvitegravir and cobicistat (COMPLERA™ or EVIPLERA™); efavirenz (optionally, SUSTIVA™), emtricitabine and tenofovir (or ATRIPLA™); lamivudine, nevirapine and stavudine (for example, TRIOMUNE™); atazanavir (or REYATAZ™) and cobicistat (for example, EVOTAZ™); lamivudine and raltegravir (for example, DUTREBIS™); lamivudine and dolutegravir (or DOVATO™); doravirine, lamivudine and tenofovir (for example, DELSTRIGO™); or lamivudine, zidovudine and nevirapine (for example, CUOVIR-N™), and optionally the anti-viral drug or drug combination comprises daclatasvir (optionally DAKLINZA™);
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a viral, or a coronavirus or a COVID-19, protease inhibitor, wherein optionally the protease inhibitor comprises: ASC09 (CAS registry no. 1000287-05-7) (Janssen Research and Development, LLC), ritonavir (optionally NORVIR™) or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir, cobicistat or darunavir and cobicistat, or PF-07321332, or nirmatrelvir, or the combination of nirmatrelvir and ritonavir, or PAXLOVID™, PF-07304814 or PF-008335231 (Pfizer), or remdesivir (for example, GS-5734™, Gilead Sciences) or ritonavir (optionally NORVIR™) in combination with the PF-07321332, or nirmatrelvir, or the combination of nirmatrelvir and ritonavir, or PAXLOVID™, PF-07304814 or PF-008335231 (Pfizer) optionally as an oral formulation, optionally as a tablet, geltab or capsule,

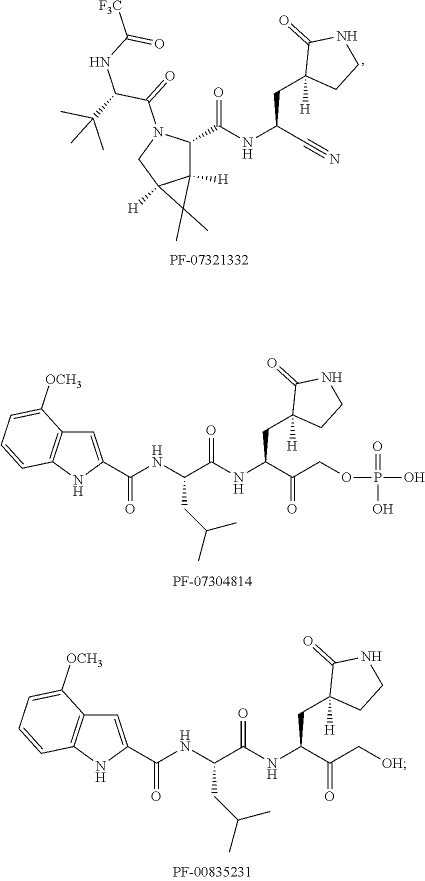 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C ; COc1cccc2[nH]c(C(=O)N[C@@H](CC(C)C)C(=O)N[C@@H](C[C@@H]3CCNC3=O)C(=O)COP(=O)(O)O)cc12 ; COc1cccc2[nH]c(C(=O)N[C@@H](CC(C)C)C(=O)N[C@@H](C[C@@H]3CCNC3=O)C(=O)CO)cc12]

a thiazolide class drug, optionally nitazoxanide (or ALINIA™, NIZONIDE™) or tizoxanide (or 2-Hydroxy-N-(5-nitro-2-thiazolyl)benzamide);
a blood clot inhibiting drug such as aspirin, warfarin (or COUMADIN™) or rivaroxaban (or XARELTO™);
lopinavir, ritonavir (optionally NORVIR™) or the combination of lopinavir and ritonavir (or KALETRA™, ALTERA™, ALUVIA™, KALMELTREX, or LOPIIVIUNE™), opaganib (or YELIVA™), oseltamivir (or TAMIFLU™), and/or zanamivir (or RELENZA™);
an inhibitor or S-phase kinase-associated protein 2 (SKP2), or dioscin, or niclosamide, or NICLOCIDE™, FENASAL™, or PHENASAL™;
a tyrosine kinase inhibitor (TKi), wherein the TKi comprises: masitinib (or MASIVET™, or KINAVET™); or imatinib (or GLEEVEC™, GLIVEC™); or gefitinib (or IRESSA™), or erlotinib (or TARCEVA™), or dasatinib (or SPRYCEL™, DASANIX™);
ribavirin or tribavirin (or COPEGUS™, REBETOL™, or VIRAXOLE™), interferon beta 1b, or a combination of ribavirin and interferon beta, or a combination of lopinavir and ritonavir (optionally NORVIR™) and interferon-beta-1b;
a nucleoside analog reverse-transcriptase inhibitor (NRTI) (optionally abacavir, or ZIAGEN™), acyclovir (or ZOVIRAX™), optionally ACICLOVIR™), adefovir (optionally HEPSERA™), amantadine (optionally GOCOVRI™, SYMADINE™, SYMMETREL™), rintatolimod (or AMPLIGEN™), amprenavir (optionally, AGENERASE™), aprepitant (or EMEND™), umifenovir (or ARBIDOL™), atazanavir (or REYATAZ™), tenofovir or tenofovir disoproxil (or VIREAD™), a combination of efavirenz and emtricitabine and tenofovir (or ATRIPLA™), balavir, baloxavir marboxil (XOFLUZA™), bepotastine (or TALION™, BEPREVE™), bevirimat, bictegravir, biktarvy, brilacidin, cidofovir, caspofungin, lamivudine and zidovudine (optionally, COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir (or VIRACEPT™), nevirapine, nexavir, nitazoxanide, norvir, a nucleoside analogue (optionally brincidofovir, didanosine, favipiravir (also known as T-705, AVIGAN™, or favilavir, Toyama Chemical, Fujifilm, Japan), vidarabine, galidesivir (optionally, BCX4430, IMMUCILLIN-ATM), remdesivir (optionally, GS-5734TH, Gilead Sciences), cytarabine, gemcitabine, emtricitabine, lamivudine, zalcitabine, entecavir, stavudine, telbivudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir (optionally NORVIR™), saquinavir, sofosbuvir, stavudine, telaprevir, tegobuv, tenofovir (optionally tenofovir alafenamide, tenofovir disoproxil or VIREAD™), tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™) or any combination thereof;
fenofibrate, or TRICOR™, FENOBRAT™, FENOGLIDE™, or LIPOFEN™, or a combination of fenofibrate and simvastatin, or CHOLIB™;
suramin, or ANTRYPOL™, BAYER 305™, or GERMANIN™;
a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™ Glenmark Pharmaceuticals),
   wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 5 to 15 days, or for about 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days,
   and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin),
   and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin) and an antibiotic, and optionally the antibiotic comprises azithromycin, minocycline, amoxicillin, niclosamide, nitazoxanide, hydroxychloroquine or doxycycline), and optionally the synthetic nucleoside analog or derivative, avermectin class drug, and antibiotic are administered together or as separate formulations, and optionally are administered every one, two, three, four or five weeks for between about one month and one year or more;
   and optionally molnuvpiravir, ivermectin and hydroxychloroquine are administered together or as separate formulations, and optionally are administered every one, two, three, four or five weeks for between about one month and one year or more;
   and optionally the synthetic nucleoside analog or derivative (optionally N4-hydroxycytidine, or the prodrug of N4-hydroxycytidine, optionally molnuvpiravir or favipiravir), and antibiotic (optionally doxycycline or hydroxychloroquine) is administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D),
   and optionally the synthetic nucleoside analog or derivative, or N4-hydroxycytidine, or the prodrug of N4-hydroxycytidine, optionally molnuvpiravir or favipiravir, is administered with at least two antibiotics (optionally the at least two antibiotics comprise azithromycin, minocycline, amoxicillin, niclosamide, nitazoxanide, hydroxychloroquine and/or doxycycline), and a zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
an antibody or antibody or vaccine therapy for treating, preventing or ameliorating a microbial or a viral infection (optionally a coronavirus infection, optionally a COVID-19 infection) or a microbial infection (optionally a protozoan, helminthiasis, insect and/or parasitic infection), and optionally the antibody comprises a monoclonal antibody, and optionally the monoclonal antibody comprises sotrovimab (GlaxoSmithKline and Vir Biotechnology), or casirivimab, imdevimab or casirivimab and imdevimab (REGEN-COV™) (Regeneron), or bamlanivimab oretesevimab or bamlanivimab and etesevimab (Junshi Biosciences), or tocilizumab or ACTEMRA™ or ROACTEMRA™ (Hoffmann-La Roche),
   and optionally the antibody or vaccine therapy comprises tozinameran or COMIRNATY™ (Pfizer), or elasomeran or SPIKEVAX™ (Moderna), or SPUTNIK V™ or Gam-COVID-Vac (Gamaleya Research Institute), or AZD1222 or COVISHIELD™ or VAXZEVRIA™ (Oxford-AstraZeneca),
   and optionally the antibody or antibody therapy comprises or is contained in a convalescent sera or plasma,
wherein optionally any of these combinations is administered very 2, 3, 4, 5, 6, 7, 8, 9 or 10 or more days for between about 1 month and one year or more; and/or
or any combination thereof.
14. The method of any one of the preceding forms, wherein a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) are administered:
(a) once a month; or
(b) for the first four, five, six or seven days of treatment an avermectin class drug (optionally ivermectin) is given at about 24 mg per day or between about 20 to 30 mg per day, doxycycline or azithromycin is given at about 100 mg per day or between about 50 and 150 mg per day, clofazimine is given about 100 mg per day or between about 50 and 150 mg per day, and zinc, zinc salt or zinc chelate (optionally a zinc sulphate, acetate, gluconate or picolinate, or zinc oxide nanoparticles) is administered at a dosage of between about 25 mg to 100 mg per day, or about 50 mg per day),
and after this initial first four, five, six or seven days of treatment a once a month maintenance regimen of an avermectin class drug (optionally ivermectin) dosaged at between about 60 to 80 mg, or about 60 mg, clofazimine dosaged at about 100 mg or between about 50 to 150 mg, doxycycline or azithromycin dosaged at about 100 mg or between about 50 to 150 mg, and zinc, zinc salt or zinc chelate (optionally a zinc sulphate, acetate, gluconate or picolinate, or zinc oxide nanoparticles) is administered at a dosage of between about 25 mg to 100 mg per day, or about 50 mg per day) is given,
and optionally the avermectin class drug (optionally ivermectin), clofazimine, doxycycline or azithromycin and zinc are formulated and administered in separate dosage units (optionally geltabs, tablets, capsules), or the avermectin class drug (optionally ivermectin), clofazimine, doxycycline or azithromycin and zinc, zinc salt or zinc chelate are formulated and administered in one unit dosage (optionally all in one a geltab, tablet, capsule).
15. The method of any one of the preceding forms, wherein the individual in need thereof suffers from long term effects, or chronic effects or symptoms, of a viral infection, or the individual in need thereof has not fully recovered from the viral infection weeks or even months after first experiencing symptoms, or the individual in need thereof experiences continuous symptoms for weeks or months after being first diagnosed or treated with the viral infection, or the individual in need thereof feels better for weeks, then relapses with old or new symptoms,
and optionally the medication or the drug combination is administered to prevent a so-called “long-hauler” syndrome, or to treat or prevent continuous symptoms for weeks or months, or to prevent or treat relapsing with old or new symptoms,
wherein optionally the viral infection is a COVID-19 infection.
16. The method of any one of the preceding forms, wherein the synthetic nucleoside analog or derivative, or N4-hydroxycytidine, or the prodrug of N4-hydroxycytidine, optionally molnuvpiravir or favipiravir, is administered with an antibiotic (optionally the antibiotic comprises azithromycin, minocycline, amoxicillin, niclosamide, nitazoxanide, hydroxychloroquine or doxycycline), optionally also administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
and optionally the synthetic nucleoside analog or derivative, or N4-hydroxycytidine, or the prodrug of N4-hydroxycytidine, optionally molnuvpiravir or favipiravir, is administered with azithromycin and/or hydroxychloroquine and/or doxycycline with an avermectin class drug optionally comprising: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
and optionally the synthetic nucleoside analog or derivative, or N4-hydroxycytidine, or the prodrug of N4-hydroxycytidine, optionally molnuvpiravir or favipiravir, is administered with opinavir, ritonavir (optionally NORVIR™), or the combination lopinavir and ritonavir (or KALETRA™, ALTERA™, ALUVIA™, KALMELTREX or LOPIMUNE™), opaganib (or YELIVA™), oseltamivir (or TAMIFLU™), and/or zanamivir (or RELENZA′).

In another aspect, forms of the invention may include the following:
1. A drug or drug (or therapeutic) combination or composition comprising:
(a)
   (i) a loading dosage comprising an avermectin class drug (optionally ivermectin) in a dosage of:
   (1) about 300 μg/kg to 30 mg/kg (or 30 mg per 2.2 pounds (lb)) or about 18 mg to 1800 mg in a 60 kg (about 132 lb), or a loading dose of the avermectin class drug (optionally ivermectin) of between about 300 μg/kg to 30 to 60 mg/kg or between about 18 mg to about 1200 mg or 1600 mg to 1800 mg in a 60 kg (about 132 lb) person, or between about 300 μgm (mcg) to 40 mg/kg or 70 mg/kg, or a dosage of between about 120 mg to 280 mg to about 1600 to 1800 mg for an adult; or
   (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; and
   (ii) after administration of the loading dosage of (i), administering a maintenance dosage of ivermectin of between about 20 mcg/kg (μ/kg) to 5000 mcg/kg (μ/kg) or between about 200 to 2000 mcg/kg (μ/kg) per dose, where 200 mcg/kg is equivalent to a 12 mg dosage in a 60 kg adult, and 2000 mcg/kg is equivalent to 120 mg per dose;
(b) a drug, a formulation or a therapeutic combination of drugs comprising an avermectin class drug (optionally ivermectin) at a dosage of:
   (1) about 300 μg/kg to 30 mg/kg (or 30 mg per 2.2 pounds (lb)) or about 18 mg to 1800 mg in a 60 kg (about 132 lb), or at a loading dose of the avermectin class drug (optionally ivermectin) of between about 300 μg/kg to 30 to 60 mg/kg or between about 18 mg to about 1200 mg or 1600 mg to 1800 mg in a 60 kg (about 132 lb) person, or between about 300 μgm (mcg) to 40 mg/kg or 70 mg/kg, or a dosage of between about 120 mg to 280 mg to about 1600 to 1800 mg for an adult, or
   (2) between about 18 to 50 mg, or about 18 mg, 24 mg, 30 mg, 36 mg or 40 mg, or between about 50 mg to 100 mg, or 60 to 120 mg up to about 1600 to 1800 mg
for an adult;

(c) a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™, Glenmark Pharmaceuticals),
wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days;
(d) an anti-androgen drug, and optionally the anti-androgen drug is bicalutamide, optionally CASODEX™, or dutasteride (or AVODART™),
   and optionally the anti-androgen drug is a nonsteroidal anti-androgen (NSAA) or an androgen receptor (AR) antagonist, and optionally the NSAA or AR antagonist comprises proxalutamide (or its developmental name GT-0918) (Suzhou Kintor Pharmaceuticals, Inc., a subsidiary of Kintor Pharmaceutical Limited), or flutamide (or niftolide, or EULEXIN™), or bicalutamide (or CASODEX™) or enzalutamide (or XTANDI™),
   and optionally the anti-androgen drug comprises a 5α-reductase inhibitor, and optionally the 5α-reductase inhibitor comprises finasteride (or PROSCAR™, PROPECIA™ or FINIDE™),
   and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
   and optionally the anti-androgen drug, or NSAA, or bicalutamide, proxalutamide, flutamide or niftolide, bicalutamide, enzalutamide or dutasteride, is administered with an avermectin class drug, or ivermectin, optionally also administered with hydroxychloroquine, zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A),
   and optionally the anti-androgen drug, or NSAA, or bicalutamide, proxalutamide, flutamide or niftolide, bicalutamide, enzalutamide or dutasteride, is administered with colchicine (or COLCRYS™, MITIGARE™), and optionally also zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A),
   and optionally the anti-androgen drug, or NSAA, or bicalutamide, proxalutamide, flutamide or niftolide, bicalutamide, enzalutamide or dutasteride, is administered with an antibiotic (optionally azithromycin or doxycycline), and optionally also zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A), and optionally also with hydroxychloroquine;
(e) an anti-malarial drug, wherein optionally the anti-malarial drug comprises mefloquine (or LARIAM™, MEPHAQUIN™, or MEFLIAM™), wherein optionally the mefloquine is formulated for oral administration, optionally in tablet or capsule form, optionally as 200 mg, 250 mg or 300 mg tablets;
(f) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof,
(g) an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
and optionally the DDS system comprises: a polyether ester urethane comprising 65% D, L-lactide, 19% polyethylene glycol, and 16% glycolide interlinked with an aliphatic di-isocyanate, or comprises VISCOPRENE™,
and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously,
(h) a nicotinic antagonist, a dopamine agonist or a noncompetitive N-Methyl-D-aspartate (NMDA) antagonist, optionally amantadine, or GOCOVRI™, or SYMADINE™, or SYMMETREL™, optionally dosaged at between about 100 to 200 mg per dose, optionally formulated as tablets or capsules, or
(i) a mitochondrial sensitizer, optionally proguanil or chlorguanide (or PALUDRINE™), or a malarial cytochrome bc1 complex inhibitor, optionally atovaquone (or MEPRON™), or a combination of proguanil and atovaquone (or MALARONE™), and/or
(j) a drug combination or therapeutic regimen comprising any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i)
for use in preventing, or substantially preventing, decreasing the chances of having any adverse effects from, decreasing the severity of adverse effects from, or treating or ameliorating a viral infection or a microbial infection, or a protozoan, helminthiasis, insect and/or parasitic infection, in an individual in need thereof.
2. The drug or drug (or therapeutic) combination or composition for use of form 1, wherein the avermectin class drug comprises: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin.
3. The drug or drug (or therapeutic) combination or composition for use of form 1 or form 2, wherein the drug or drug combination is administered to prevent or substantially prevent, or to treat or ameliorate, or decrease the severity of symptoms or pathology of:
   a viral infection, optionally a coronavirus, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection,
   a coronavirus infection, optionally a COVID-19 infection, optionally a COVID-19 variant infection, wherein optionally the COVID-19 variant is a delta or an omicron variant, or the coronavirus infection comprises a Middle East respiratory syndrome virus (MERS-CoV) infection;
   malaria that can be caused by a parasite of the genus Plasmodium (optionally P. vivax, P. falciparum, P. malariae, P. ovale, or P. knowlesi);
   dengue fever or dengue shock syndrome that can be caused by a virus of the Flaviviridae family or a dengue virus;
   hepatitis or hepatocellular carcinoma associated with viral hepatitis that can be caused by a virus of the Flaviviridae family or a virus of the genus Hepacivirus or Hepacivirus C virus or hepatitis C;
   filariasis, leprosy or streptocerciasis that can be caused by a parasite of the superfamily Filarioidea (optionally Brugia malayi, Brugia timori, Wuchereria bancrofti, Loa loa, Mansonella streptocerca, Mansonella ozzardi, or Mansonella perstans);
   leprosy that can be caused by a parasite of the genus Mycobacterium (optionally M leprae or M. lepromatosis);
   river blindness or onchocerciasis that can be caused by parasitic worms such as parasites of the genus Onchocerca (optionally O. volvulus);
   hookworm or roundworm infections that can be caused by parasites of the genus Ancylostoma (optionally A. duodenale or A. ceylanicum) or Necator (optionally N. americanus);
   trichuriasis or whipworm infection that can be caused by a parasite of the genus Trichuris (optionally T. trichiura); roundworm or an Ascaris infection that can be caused by Ascaris lumbricoides;
   mite-carried infections such as scabies that can be caused by the parasite of the genus Sarcoptes (optionally S. scabiei);
   infections such as typhus caused by lice or parasites of the order Phthiraptera (optionally Pediculus humanus capitis);
   enterobiasis that can be caused by pinworm or parasites of the genus Enterobius (optionally E. vermicularis); and/or
   pulicosis or infections cause by fleas or insects of the order Siphonaptera or of the genus Pulex (optionally P. irritans).
4. The drug or drug (or therapeutic) combination or composition for use of any one of forms 1 to 3, wherein the loading dose of the avermectin class drug (optionally ivermectin) of between is about 15 to 150 mg/kg, or is about 18, 24, 30, 35, 40, 35, 50, 55, 60, 65, 70, 75, 80, 85, 90, 95, 100, 110 or 120 or more mg/kg.
5. The drug or drug (or therapeutic) combination or composition for use of any one of the preceding forms, wherein the loading dosage is given once, or periodically, optionally every 2, 3, 4, 5, 6, 7, 8, 9, 10, 11, or 12 or more days.
6. The drug or drug (or therapeutic) combination or composition for use of any one of the preceding forms, wherein the maintenance dosage of (a)(ii) is administered 1, 2, 3, 4, 5, 6, 7, 8, 9, 10, 11, 12, 13 or 14 days, or every 3 weeks or every month or every two months or longer after the first loading dosage.
7. The drug or drug (or therapeutic) combination or composition for use of any one of the preceding forms, wherein the maintenance dosage of (a)(ii) is administered every 1, 2, 3, 4, 5, 6, 7, 8, 9, 10, 11, 12, 13 or 14 days, every 3 weeks, or monthly, over the 4 to 8 weeks, 6 to 10 weeks, 8 to 12 weeks, 10 to 20 weeks, 15 to 30 weeks or 20 to 52 weeks, or more, after the initial or loading dose is given.
8. The drug or drug (or therapeutic) combination or composition for use of any one of the preceding forms, wherein: an antibiotic or anti-viral is administered with the loading dosage of the avermectin class drug (optionally ivermectin); zinc or a zinc salt or zinc chelate is administered with the loading dosage of the avermectin class drug (optionally ivermectin); or zinc or zinc chelate or a zinc salt and an antibiotic is administered with the loading dosage of the avermectin class drug (optionally ivermectin),
and optionally a drug combination, optionally formulated as one formulation (for example, as a tablet capsule) comprises: ivermectin, doxycycline and zinc chelate, or comprises: ivermectin 12 mg, doxycycline 100 mg and zinc chelate 25 mg.
9. The drug or drug (or therapeutic) combination or composition for use of form 8, wherein the antibiotic comprises doxycycline, azithromycin or hydroxychloroquine (HCQ).
10. The drug or drug (or therapeutic) combination or composition for use of any one of the preceding forms, wherein: an antibiotic or anti-viral is administered with the maintenance dose of the avermectin class drug (optionally ivermectin); zinc or a zinc salt or zinc chelate is administered with the maintenance dosage of the avermectin class drug (optionally ivermectin); or zinc or a zinc salt or zinc chelate and an antibiotic or anti-viral is administered with the maintenance dosage of the avermectin class drug (optionally ivermectin).
11. The drug or drug (or therapeutic) combination or composition for use of form 10, wherein the antibiotic comprises doxycycline, azithromycin or hydroxychloroquine (HCQ).
12. The drug or drug (or therapeutic) combination or composition for use of any one of the preceding forms, wherein an additional drug is or drugs, or therapy, is or are administered with the loading dose and/or the maintenance dose of the avermectin class drug (optionally ivermectin), or before the loading dose and/or the maintenance dose, or any time between administration of the loading dose and the maintenance dose.
13. The drug or drug (or therapeutic) combination or composition for use of any one of the preceding forms, wherein an additional drug or therapy, is or are administered with the drug or drug combination of (a), (b), (c), (d), (e), (f), (g), (h) or (i), or any one or more of the following is administered with the drug combination or therapeutic regimen of any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i) of form 1:
   a thiazolide class drug, optionally nitazoxanide (or Alinia™, Nizonide™) or tizoxanide (or 2-Hydroxy-N-(5-nitro-2-thiazolyl)benzamide);
   molnupiravir, optionally co-administered with and/or formulated with an avermectin class drug (optionally ivermectin), an antibiotic (optionally doxycycline or azithromycin) and/or zinc, zinc salt or zinc chelate, or co-administered with and/or formulated with ivermectin, hydroxychloroquine, an antibiotic (optionally doxycycline or azithromycin) and/or zinc, zinc salt or zinc chelate;
   a mucolytic therapy or drug, optionally acetylcysteine, ambroxol, bromhexine (or BISOLVON™), carbocisteine, erdosteine, mecysteine or dornase alfa, or an expectorant, optionally guaifenesin;
   an H2 antagonist, or H2RA, or H2 blockers, or a compound, drug or formulation that decreases stomach acid production or decreases stomach pH, wherein optionally the compound, drug or formulation comprises famotidine (or PEPCID™), ranitidine (or ZANTAC™), nizatidine (or AXID™ or TAZAC™), roxatidine acetate, lafutidine, or cimetidine (or TAGAMET™), and optionally the famotidine is administered at a dosage of between about 10 to 60 mg per day, or between about 20 to 40 mg per day;
   a dendrimer, optionally astodrimer sodium (Starpharma, Melbourne, Australia);
   an antihistamine class drug such as azelastine, or ASTELIN™, OPTIVAR™, ALLERGODIL™, bepotastine (or TALION™, BEPREVE™) brompheniramine, fexofenadine or ALLEGRA™, pheniramine or AVIL™, or chlorpheniramine;
   a selective serotonin reuptake inhibitor (SSRI) class drug, optionally fluvoxamine, or LUVOX™, FAVERIN™, FLUVOXIN™;
   a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™ or ronifibrate, or simfibrate or CHOLESOLVIN™, or any combination thereof, clofazimine, or LAMPENE™, optionally dosaged at about 100 mg per day, or between about 50 mg and 150 mg per day, and optionally also including colchicine;
   clofazimine, or LAMPENE™, optionally dosaged at about 100 mg per day, or between about 50 mg and 150 mg per day, and chloroquine (or ARALEN™), chloroquine phosphate, chloroquine diphosphate and/or hydroxychloroquine (HCQ) (optionally, PLAQUENIL™), optionally also comprising zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally also including administration of zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine,
   a combination of clofazimine (optionally dosaged at about 100 mg or 150 mg per day, or between about 50 mg and 200 mg per day), fluvoxamine, and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), fluvoxamine and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally further comprising zinc, zinc salt or zinc chelate (optionally a zinc sulphate, acetate, gluconate or picolinate, or zinc oxide nanoparticles, optionally at a dosage of between about 1 mg to 250 mg), and optionally also including colchicine;
   hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);
   chloroquine (or ARALEN™), chloroquine phosphate, chloroquine diphosphate and/or hydroxychloroquine (HCQ) (optionally, PLAQUENIL™);
   a corticosteroid or glucocorticoid class drug such as such as ciclesonide (or Alvesco™, Omnaris™, Omniair™, Zetonna™ or Alvesco™), budesonide (optionally RHINOCORT™ or PULMICORT™), prednisolone (or ORAPRED™), methyl-prednisolone, prednisone (or DELTASONE™ or ORASONE™) or hydrocortisone (or CORTEF™), or a selective estrogen receptor modulator (SERM), or toremifene (or Fareston™), or clomifene or clomiphene (or CLOMID™, SEROPHENE™), wherein optionally the mode of administration for the corticosteroid or glucocorticoid class drug (optionally ciclesonide) is by inhalation (i.e., they are inhaled);
   a hydrocortisone or cortisol (optionally CORTEF™, SOLUCORTEF™), optionally hydrocortisone sodium succinate or hydrocortisone acetate or dexamethasome (optionally Dextenza™, Ozurdex™, Neofordex™),
      and optionally the corticosteroid or glucocorticoid class drug (for example budesonide or ciclesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,
      and optionally the corticosteroid or glucocorticoid class drug (optionally budesonide or ciclesonide) is formulated as a powder or for administration in an inhaler or by nasal spray, or for rectal administration,
      and optionally the corticosteroid or glucocorticoid class drug (for example, budesonide or ciclesonide) is administered together with or in combination with 10 mg to 80 mg, an antibiotic (optionally azithromycin or a tetracycline class drug,
      wherein optionally the tetracycline class drug comprises doxycycline, or DORYX™, DOXYHEXA™, DOXYLIN™), zinc, zinc salt or zinc chelate and/or a vitamin (optionally vitamin D or calcifediol, D2 (or ergocalciferol), D3 (or cholecalciferol), C, E, B12, B6);
   an anti-androgen drug, and optionally the anti-androgen drug is bicalutamide, optionally CASODEX™, or dutasteride (or AVODART™),
      and optionally the anti-androgen drug is a nonsteroidal anti-androgen (NSAA) or an androgen receptor (AR) antagonist, and optionally the NSAA or AR antagonist comprises proxalutamide (or its developmental name GT-0918) (Suzhou Kintor Pharmaceuticals, Inc., a subsidiary of Kintor Pharmaceutical Limited), or flutamide (or niftolide, or EULEXIN™), or bicalutamide (or CASODEX™) or enzalutamide (or XTANDI™),
      and optionally the anti-androgen drug comprises a 5α-reductase inhibitor, and optionally the 5α-reductase inhibitor comprises finasteride (or PROSCAR™, PROPECIA™, or FINIDE™),
      and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;
   an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2),
      and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin or a tetracycline class drug, wherein optionally the tetracycline class drug comprises doxycycline, or DORYX™, DOXYHEXA™, DOXYLIN™); chloroquine (or ARALEN™), chloroquine phosphate, chloroquine diphosphate and/or hydroxychloroquine (optionally, PLAQUENIL™); zinc, zinc salt or zinc chelate; remdesivir (optionally, GS-5734™, Gilead Sciences); oseltamivir (or TAMIFLU™); and/or, hydrocortisone; or, any combination thereof;
   a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or a tetracycline tetracycline class drug, and optionally the tetracycline class drug comprises doxycycline, or DORYX™ DOXYHEXA™, DOXYLIN™;
   a dendrimer, optionally astodrimer sodium (Starpharma, Melbourne, Australia);
   an antihistamine class drug such as azelastine, or ASTELIN™, OPTIVAR™, ALLERGODIL™, brompheniramine, fexofenadine or ALLEGRA™, pheniramine or AVIL™, or chlorpheniramine;
   a selective serotonin reuptake inhibitor (SSRI) class drug, optionally fluvoxamine, or LUVOX™, FAVERIN™, FLUVOXIN™;
   a nicotinic antagonist, a dopamine agonist or a noncompetitive N-Methyl-d-aspartic acid or N-Methyl-d-aspartate (NMDA) antagonist, wherein optionally the nicotinic antagonist, dopamine agonist or noncompetitive NMDA antagonist is 1-adamantylamine or amantadine, or GOCOVRI™ SYIVIADINE™, SYMMETREL™, optionally administered or dosaged at between about 50 mg to 150 mg, or about 100 mg, or 200 mg, per day for a period of between about 7 and 21 days, or about 14 days, and optionally the nicotinic antagonist, dopamine agonist or noncompetitive NMDA antagonist is also administered or formulated with an antibiotic (optionally azithromycin or doxycycline), ivermectin, hydroxychloroquine (optionally, PLAQUENIL™) and/or zinc, zinc salt or zinc chelate (optionally zinc sulfate, optionally at (50 mg daily), and optionally the amantadine is formulated or administered at 100 mg per day for the first two days of treatment, which optionally can then be elevated to 100 mg twice daily, optionally for the next 10 days;
   an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
      and optionally the DDS system comprises: a polyether ester urethane comprising 65% d,l-lactide, 19% polyethylene glycol, and 16% glycolide interlinked with an aliphatic di-isocyanate, or comprises VISCOPRENE™
      and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously;
   an immunosuppressive drug, wherein optionally the immunosuppressive drug comprises tocilizumab or atlizumab, or Actemra™, or RoActemra™, or a calcineurin inhibitor (CNI), wherein the CNI comprises ciclosporin (or cyclosporine or cyclosporin), or Neoral™, or Sandimmune™, or tacrolimus, or Protopic™, or Prograf™, and optionally the immunosuppressive drug is also administered or formulated with an antibiotic (optionally azithromycin or doxycycline), ivermectin, hydroxychloroquine (optionally, PLAQUENIL™) and/or zinc, zinc salt or zinc chelate (optionally zinc sulfate, optionally at (50 mg daily),
      and optionally the calcineurin inhibitor (CNI), wherein the CNI comprises ciclosporin (or cyclosporine or cyclosporin) is formulated combination of CNI (optionally cyclosporine) at a dose of 3 mg/kg (180 mg daily) together with 12 mg ivermectin once, and optionally also plus zinc 50 mg base and doxycycline 100 mg bid, optionally all for 10 days;
   a protein kinase inhibitor, wherein optionally the protein kinase inhibitor is a p38 mitogen-activated protein kinase inhibitor, or ralimetinib, and optionally the protein kinase inhibitor is also administered or formulated with an antibiotic (optionally azithromycin or doxycycline), ivermectin, hydroxychloroquine (optionally, PLAQUENIL™) and/or zinc or any zinc salt or zinc chelate (optionally zinc sulfate, optionally at (50 mg daily);
   an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally (optionally a synthetic glucocorticoid, hydrocortisone, dexamethasone (or DEXTENZA™, OZURDEX™, or NEOFORDEX™) or cortisol, or CORTEF™), plitidepsin or dehydrodidemnin B, or APLIDIN™, or a nonsteroidal anti-inflammatory drug (NSAID), wherein optionally the NSAID comprises indomethacin (or indomethacin) or INDOCID™ or INDOCIN™, or naproxen, or NAPROSYN™ or ALEVE™, or a cyclooxygenase inhibitor, or a COX-1 or an COX-2 inhibitor, or aspirin, or ibuprofen or ADVIL™ MOTRIN™ or NUROFEN™, or celecoxib or CELEBREX™, or parecoxib or DYNASTAT™, or etoricoxib or ARCOXIA™
      and optionally the anti-inflammatory therapy or anti-inflammatory therapy drug is also administered or formulated with an antibiotic (optionally azithromycin or doxycycline), ivermectin, hydroxychloroquine (optionally, PLAQUENIL™) and/or zinc or any zinc salt or zinc chelate (optionally zinc sulfate, optionally at (50 mg daily),
      and optionally opaganib, or YELIVA™, or opaganib, or YELIVA™ administered or formulated together with an oral and/or inhaled or aerosol chloroquine (or ARALEN™), chloroquine phosphate, chloroquine diphosphate and/or hydroxychloroquine (optionally, PLAQUENIL™),
      and optionally the opaganib or YELIVA™ is formulated or administered at a dosage of QD (once a day), bid (twice a day) or tid (three times a day) at a dosage of between about 100 to 600 mg per day or per dosage, or at about 100, 200, 300, 400, 500 or 600 mg per day or per dosage,
      and optionally the opaganib, or YELIVA™ is also administered or formulated with an antibiotic (optionally azithromycin or doxycycline), ivermectin (optionally at 12 mg ivermectin, optionally administered on days 1, 3, 6 and 8), hydroxychloroquine (optionally, PLAQUENIL™) and/or zinc or any zinc salt or zinc chelate (optionally zinc sulfate, optionally at (50 mg daily);
   a calcium channel blocker, or verapamil (or ISOPTIN™, CALAN™), or a voltage gated potassium (KCNH2) channel or a voltage gated calcium channel (CACNA2D2) blocker, or amiodarone (or CORDARONE™, NEXTERONE™);
   a suramin, or ANTRYPOL™, BAYER 305™, or GERMANIN™;
   a PPAR agonist, optionally fenofibrate, or TRICOR™, FENOBRAT™, FENOGLIDE™, or LIPOFEN™, and optionally the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™ or ronifibrate, or simfibrate or CHOLESOLVIN™, or any combination thereof, or a combination of fenofibrate and simvastatin, or CHOLIB™;
   a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™ Glenmark Pharmaceuticals),
      wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days,
      and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin),
      and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin) with an antibiotic, and optionally the antibiotic comprises azithromycin, minocycline, amoxicillin, niclosamide, nitazoxanide, hydroxychloroquine or doxycycline), and optionally the synthetic nucleoside analog or derivative, avermectin class drug, and antibiotic are administered together or as separate formulations, and optionally are administered every one, two, three, four or five weeks for between about one month and one year or more;
      and optionally molnuvpiravir, ivermectin and hydroxychloroquine are administered together or as separate formulations, and optionally are administered every one, two, three, four or five weeks for between about one month and one year or more;
      and optionally the synthetic nucleoside analog or derivative (optionally N4-hydroxycytidine, or the prodrug of N4-hydroxycytidine, optionally molnuvpiravir or favipiravir), and antibiotic (optionally doxycycline or hydroxychloroquine) is administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D),
      and optionally the synthetic nucleoside analog or derivative, or N4-hydroxycytidine, or the prodrug of N4-hydroxycytidine, optionally molnuvpiravir or favipiravir, is administered with an antibiotic (optionally the antibiotic comprises azithromycin, minocycline, amoxicillin, niclosamide, nitazoxanide, hydroxychloroquine or doxycycline), optionally also administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
         and optionally any of these combinations is administered very 2, 3, 4, 5, 6, 7, 8, 9 or 10 or more days for between about 1 month and one year or more;
   an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
   at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid);
   copper, optionally administered or formulated at a dosage of between about 1 to 200 mg per day, wherein optionally the copper is administered or formulated as cupric chloride and administered intravenously formulated at about 0.4 mg/ml;
   selenium, optionally administered as selenious acid formulated at about 65.4 mcg/ml (or μ/ml), and optionally the selenium is administered at a dosage of between about 50 to 100 optionally between about 60 to 100 μgm per day is administered to an adult, and only up to 60 μgm per day for pediatric patients;
   favipiravir (or T-705, avigan, or favilavir), optionally at 800 mg bid;
   zinc, zinc salt or zinc chelate (optionally a zinc sulphate, acetate, gluconate or picolinate) or zinc oxide nanoparticles, optionally at a dosage of between about 1 mg to 250 mg;
   colchicine, or COLCRYS™, MITIGARE™;
   at least one antibiotic or anti-viral (wherein optionally the antibiotic is doxycycline (optionally, DORYX™, DOXYHEXA™, DOXYLIN™) (optionally formulated or administered at a dosage of between about 25 mg to 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
   at least one anti-viral drug or medication, or anti-microbial drug, or palliative agent or drug, wherein optionally the anti-viral drug or medication, or anti-microbial drug, is or comprises efavirenz (for example, SUSTIVA™), tenofovir (optionally tenofovir alafenamide or tenofovir disoproxil, or VIREAD™), emtricitabine and tenofovir, nevirapine (or the combination efavirenz with emtricitabine and tenofovir, or ATRIPLA™), amprenavir (for example, AGENERASE™), nelfinavir (for example, VIRACEPT™) and/or remdesivir (for example, GS-5734™, Gilead Sciences), a viral RNA-dependent RNA polymerase inhibitor, optionally favipiravir (optionally AVIGAN™) or sofosbuvir (optionally SOVALDI™, SOFORAL™); or, an adenosine analog (optionally galidesivir, optionally BCX4430, INIMUCILLIN-A™),
      and optionally the anti-viral drug or medication is or comprises an anti-retroviral drug or drug combination, and optionally the anti-retroviral drug or drug combination comprises: darunavir and cobicistat (for example, REZOLSTA™ or PREZCOBIX™); atazanavir (or REYATAZ™) and cobicistat (or EVOTAZ™); abacavir, lamivudine and dolutegravir (TRIUMEQ™); tenofovir (or tenofovir disoproxil or tenofovir disoproxil, or VIREAD™, or emtricitabine) and elvitegravir and cobicistat (for example, STRIBILD™); tenofovir (or disoproxil or emtricitabine) and elvitegravir and cobicistat (COMPLERA™ or EVIPLERA™); efavirenz (optionally, SUSTIVA™), emtricitabine and tenofovir (or ATRIPLA™); lamivudine, nevirapine and stavudine (for example, TRIOMUNE™); atazanavir (or REYATAZ™) and cobicistat (for example, EVOTAZ™); lamivudine and raltegravir (for example, DUTREBIS™); lamivudine and dolutegravir (or DOVATO™); doravirine, lamivudine and tenofovir (for example, DELSTRIGO™); or lamivudine, zidovudine and nevirapine (for example, CUOVIR-N™), and optionally the anti-viral drug or drug combination comprises daclatasvir (optionally DAKLINZA™);
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a viral, or a coronavirus or a COVID-19, protease inhibitor, wherein optionally the protease inhibitor comprises: ASC09 (CAS registry no. 1000287-05-7) (Janssen Research and Development, LLC), ritonavir (optionally NORVIR™) or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir, cobicistat or darunavir and cobicistat, or PF-07321332, or nirmatrelvir, or the combination of nirmatrelvir and ritonavir, or PAXLOVID™, PF-07304814 or PF-008335231 (Pfizer), or remdesivir (for example, GS-5734™, Gilead Sciences) or ritonavir (optionally NORVIR™) in combination with the PF-07321332, or nirmatrelvir, or the combination of nirmatrelvir and ritonavir, or PAXLOVID™, PF-07304814 or PF-008335231 (Pfizer) optionally as an oral formulation, optionally as a tablet, geltab or capsule,

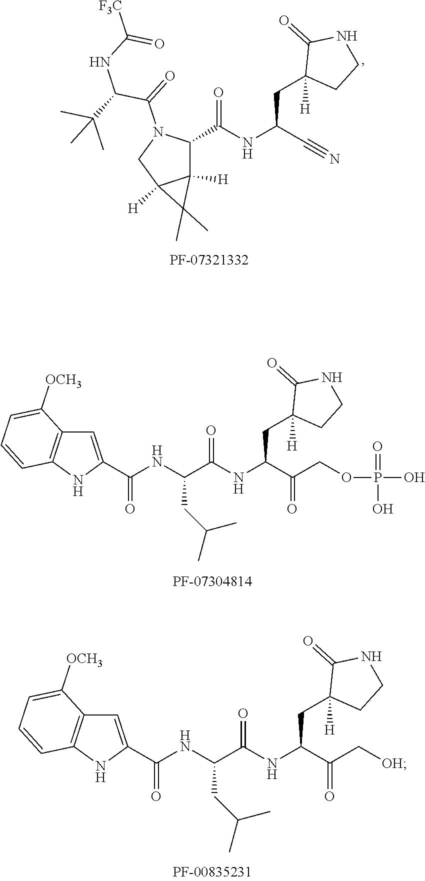 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C ; COc1cccc2[nH]c(C(=O)N[C@@H](CC(C)C)C(=O)N[C@@H](C[C@@H]3CCNC3=O)C(=O)COP(=O)(O)O)cc12 ; COc1cccc2[nH]c(C(=O)N[C@@H](CC(C)C)C(=O)N[C@@H](C[C@@H]3CCNC3=O)C(=O)CO)cc12]

a thiazolide class drug, optionally nitazoxanide (or ALINIA™, NIZONIDE™) or tizoxanide (or 2-Hydroxy-N-(5-nitro-2-thiazolyl)benzamide);
a blood clot inhibiting drug such as aspirin, warfarin (or COUMADIN™) or rivaroxaban (or XARELTO™);
lopinavir, ritonavir (optionally NORVIR™) or the combination of lopinavir and ritonavir (or KALETRA™, ALTERA™, ALUVIA™, KALMELTREX, or LOPIIVIUNE™), opaganib (or YELIVA™), oseltamivir (or TAMIFLU™), and/or zanamivir (or RELENZA™);
an inhibitor or S-phase kinase-associated protein 2 (SKP2), or dioscin, or niclosamide, or NICLOCIDE™, FENASAL™, or PHENASAL™;
a tyrosine kinase inhibitor (TKi), wherein the TKi comprises: masitinib (or MASIVET™, or KINAVET™); or imatinib (or GLEEVEC™, GLIVEC™); or gefitinib (or IRESSA™), or erlotinib (or TARCEVA™), or dasatinib (or SPRYCEL™, DASANIX™);
ribavirin or tribavirin (COPEGUS™, REBETOL™, or VIRAXOLE™), interferon beta 1b, or a combination of ribavirin and interferon beta, or a combination of lopinavir and ritonavir (optionally NORVIR™) and interferon-beta-1b;
a nucleoside analog reverse-transcriptase inhibitor (NRTI) (optionally abacavir, or ZIAGEN™), acyclovir (or ZOVIRAX™), optionally ACICLOVIR™), adefovir (optionally HEPSERA™), amantadine (optionally GOCOVRI™, SYMADINE™, SYMMETREL™), rintatolimod (or AMPLIGEN™), amprenavir (optionally, AGENERASE™), aprepitant (or EMEND™), umifenovir (or ARBIDOL™), atazanavir (or REYATAZ™), tenofovir or tenofovir disoproxil (or VIREAD™), a combination of efavirenz and emtricitabine and tenofovir (or ATRIPLA™), balavir, baloxavir marboxil (XOFLUZA™), bepotastine (or TALION™, BEPREVE™), bevirimat, bictegravir, biktarvy, brilacidin, cidofovir, caspofungin, lamivudine and zidovudine (optionally, COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir (or VIRACEPT™), nevirapine, nexavir, nitazoxanide, norvir, a nucleoside analogue (optionally brincidofovir, didanosine, favipiravir (also known as T-705, AVIGAN™, or favilavir, Toyama Chemical, Fujifilm, Japan), vidarabine, galidesivir (optionally, BCX4430, IMMUCILLIN-ATM), remdesivir (optionally, GS-5734TH, Gilead Sciences), cytarabine, gemcitabine, emtricitabine, lamivudine, zalcitabine, entecavir, stavudine, telbivudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir (optionally NORVIR™), saquinavir, sofosbuvir, stavudine, telaprevir, tegobuv, tenofovir (optionally tenofovir alafenamide, tenofovir disoproxil or VIREAD™), tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™) or any combination thereof;
fenofibrate, or TRICOR™, FENOBRAT™, FENOGLIDE™, or LIPOFEN™, or a combination of fenofibrate and simvastatin, or CHOLIB™;
suramin, or ANTRYPOL™, BAYER 305™, or GERMANIN™;
a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™ Glenmark Pharmaceuticals),
   wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 5 to 15 days, or for about 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days,
   and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin),
   and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin) and an antibiotic, and optionally the antibiotic comprises azithromycin, minocycline, amoxicillin, niclosamide, nitazoxanide, hydroxychloroquine or doxycycline), and optionally the synthetic nucleoside analog or derivative, avermectin class drug, and antibiotic are administered together or as separate formulations, and optionally are administered every one, two, three, four or five weeks for between about one month and one year or more;
   and optionally molnuvpiravir, ivermectin and hydroxychloroquine are administered together or as separate formulations, and optionally are administered every one, two, three, four or five weeks for between about one month and one year or more;
   and optionally the synthetic nucleoside analog or derivative (optionally N4-hydroxycytidine, or the prodrug of N4-hydroxycytidine, optionally molnuvpiravir or favipiravir), and antibiotic (optionally doxycycline or hydroxychloroquine) is administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D),
   and optionally the synthetic nucleoside analog or derivative, or N4-hydroxycytidine, or the prodrug of N4-hydroxycytidine, optionally molnuvpiravir or favipiravir, is administered with at least two antibiotics (optionally the at least two antibiotics comprise azithromycin, minocycline, amoxicillin, niclosamide, nitazoxanide, hydroxychloroquine and/or doxycycline), and a zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
an antibody or antibody or vaccine therapy for treating, preventing or ameliorating a microbial or a viral infection (optionally a coronavirus infection, optionally a COVID-19 infection) or a microbial infection (optionally a protozoan, helminthiasis, insect and/or parasitic infection), and optionally the antibody comprises a monoclonal antibody, and optionally the monoclonal antibody comprises sotrovimab (GlaxoSmithKline and Vir Biotechnology), or casirivimab, imdevimab or casirivimab and imdevimab (REGEN-COV™) (Regeneron), or bamlanivimab oretesevimab or bamlanivimab and etesevimab (Junshi Biosciences), or tocilizumab or ACTEMRA™ or ROACTEMRA™ (Hoffmann-La Roche),
   and optionally the antibody or vaccine therapy comprises tozinameran or COMIRNATY™ (Pfizer), or elasomeran or SPIKEVAX™ (Moderna), or SPUTNIK V™ or Gam-COVID-Vac (Gamaleya Research Institute), or AZD1222 or COVISHIELD™ or VAXZEVRIA™ (Oxford-AstraZeneca),
   and optionally the antibody or antibody therapy comprises or is contained in a convalescent sera or plasma,
wherein optionally any of these combinations is administered very 2, 3, 4, 5, 6, 7, 8, 9 or 10 or more days for between about 1 month and one year or more; and/or
or any combination thereof.
14. The drug or drug (or therapeutic) combination or composition for use of any one of the preceding forms, wherein a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) are administered:
(a) once a month; or
(b) for the first four, five, six or seven days of treatment an avermectin class drug (optionally ivermectin) is given at about 24 mg per day or between about 20 to 30 mg per day, doxycycline or azithromycin is given at about 100 mg per day or between about 50 and 150 mg per day, clofazimine is given about 100 mg per day or between about 50 and 150 mg per day, and zinc, zinc salt or zinc chelate (optionally a zinc sulphate, acetate, gluconate or picolinate, or zinc oxide nanoparticles) is administered at a dosage of between about 25 mg to 100 mg per day, or about 50 mg per day),
and after this initial first four, five, six or seven days of treatment a once a month maintenance regimen of an avermectin class drug (optionally ivermectin) dosaged at between about 60 to 80 mg, or about 60 mg, clofazimine dosaged at about 100 mg or between about 50 to 150 mg, doxycycline or azithromycin dosaged at about 100 mg or between about 50 to 150 mg, and zinc, zinc salt or zinc chelate (optionally a zinc sulphate, acetate, gluconate or picolinate, or zinc oxide nanoparticles) is administered at a dosage of between about 25 mg to 100 mg per day, or about 50 mg per day) is given,
and optionally the avermectin class drug (optionally ivermectin), clofazimine, doxycycline or azithromycin and zinc are formulated and administered in separate dosage units (optionally geltabs, tablets, capsules), or the avermectin class drug (optionally ivermectin), clofazimine, doxycycline or azithromycin and zinc, zinc salt or zinc chelate are formulated and administered in one unit dosage (optionally all in one a geltab, tablet, capsule).
15. The drug or drug (or therapeutic) combination or composition for use of any one of the preceding forms, wherein the individual in need thereof suffers from long term effects, or chronic effects or symptoms, of a viral infection, or the individual in need thereof has not fully recovered from the viral infection weeks or even months after first experiencing symptoms, or the individual in need thereof experiences continuous symptoms for weeks or months after being first diagnosed or treated with the viral infection, or the individual in need thereof feels better for weeks, then relapses with old or new symptoms,
and optionally the medication or the drug combination is administered to prevent a so-called “long-hauler” syndrome, or to treat or prevent continuous symptoms for weeks or months, or to prevent or treat relapsing with old or new symptoms,
wherein optionally the viral infection is a CON/ID-19 infection.
16. The drug or drug (or therapeutic) combination or composition for use of any one of the preceding forms, wherein the synthetic nucleoside analog or derivative, or N4-hydroxycytidine, or the prodrug of N4-hydroxycytidine, optionally molnuvpiravir or favipiravir, is administered with an antibiotic (optionally the antibiotic comprises azithromycin, minocycline, amoxicillin, niclosamide, nitazoxanide, hydroxychloroquine or doxycycline), optionally also administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
and optionally the synthetic nucleoside analog or derivative, or N4-hydroxycytidine, or the prodrug of N4-hydroxycytidine, optionally molnuvpiravir or favipiravir, is administered with azithromycin and/or hydroxychloroquine and/or doxycycline with an avermectin class drug optionally comprising: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
and optionally the synthetic nucleoside analog or derivative, or N4-hydroxycytidine, or the prodrug of N4-hydroxycytidine, optionally molnuvpiravir or favipiravir, is administered with opinavir, ritonavir (optionally NORVIR™), or the combination lopinavir and ritonavir (or KALETRA™, ALTERA™, ALUVIA™, KALMELTREX or LOPIMUNE™), opaganib (or YELIVA™), oseltamivir (or TAMIFLU™), and/or zanamivir (or RELENZA™).

In further aspect, forms of the invention may include the following:
1. Use of a drug or drug (or therapeutic) combination or composition comprising:
   (a)
      (i) a loading dosage comprising an avermectin class drug (optionally ivermectin) in a dosage of:
      (1) about 300 μg/kg to 30 mg/kg (or 30 mg per 2.2 pounds (lb)) or about 18 mg to 1800 mg in a 60 kg (about 132 lb), or a loading dose of the avermectin class drug (optionally ivermectin) of between about 300 μg/kg to 30 to 60 mg/kg or between about 18 mg to about 1200 mg or 1600 mg to 1800 mg in a 60 kg (about 132 lb) person, or between about 300 μgm (mcg) to 40 mg/kg or 70 mg/kg, or a dosage of between about 120 mg to 280 mg to about 1600 to 1800 mg for an adult; or
      (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; and
      (ii) after administration of the loading dosage of (i), administering a maintenance dosage of ivermectin of between about 20 mcg/kg (μ/kg) to 5000 mcg/kg (μ/kg) or between about 200 to 2000 mcg/kg (μ/kg) per dose, where 200 mcg/kg is equivalent to a 12 mg dosage in a 60 kg adult, and 2000 mcg/kg is equivalent to 120 mg per dose;
   (b) a drug, a formulation or a therapeutic combination of drugs comprising an avermectin class drug (optionally ivermectin) at a dosage of:
      (1) about 300 μg/kg to 30 mg/kg (or 30 mg per 2.2 pounds (lb)) or about 18 mg to 1800 mg in a 60 kg (about 132 lb), or at a loading dose of the avermectin class drug (optionally ivermectin) of between about 300 μg/kg to 30 to 60 mg/kg or between about 18 mg to about 1200 mg or 1600 mg to 1800 mg in a 60 kg (about 132 lb) person, or between about 300 μgm (mcg) to 40 mg/kg or 70 mg/kg, or a dosage of between about 120 mg to 280 mg to about 1600 to 1800 mg for an adult, or
      (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult;
   (c) a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™, Glenmark Pharmaceuticals),
   wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days;
   (d) an anti-androgen drug, and optionally the anti-androgen drug is bicalutamide, optionally CASODEX™, or dutasteride (or AVODART™),
      and optionally the anti-androgen drug is a nonsteroidal anti-androgen (NSAA) or an androgen receptor (AR) antagonist, and optionally the NSAA or AR antagonist comprises proxalutamide (or its developmental name GT-0918) (Suzhou Kintor Pharmaceuticals, Inc., a subsidiary of Kintor Pharmaceutical Limited), or flutamide (or niftolide, or EULEXIN™), or bicalutamide (or CASODEX™) or enzalutamide (or XTANDI™),
      and optionally the anti-androgen drug comprises a 5α-reductase inhibitor, and optionally the 5α-reductase inhibitor comprises finasteride (or PROSCAR™, PROPECIA™ or FINIDE™),
      and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
      and optionally the anti-androgen drug, or NSAA, or bicalutamide, proxalutamide, flutamide or niftolide, bicalutamide, enzalutamide or dutasteride, is administered with an avermectin class drug, or ivermectin, optionally also administered with hydroxychloroquine, zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A),
      and optionally the anti-androgen drug, or NSAA, or bicalutamide, proxalutamide, flutamide or niftolide, bicalutamide, enzalutamide or dutasteride, is administered with colchicine (or COLCRYS™, MITIGARE™), and optionally also zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A),
      and optionally the anti-androgen drug, or NSAA, or bicalutamide, proxalutamide, flutamide or niftolide, bicalutamide, enzalutamide or dutasteride, is administered with an antibiotic (optionally azithromycin or doxycycline), and optionally also zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A), and optionally also with hydroxychloroquine;
   (e) an anti-malarial drug, wherein optionally the anti-malarial drug comprises mefloquine (or LARIAM™, MEPHAQUIN™, or MEFLIAM™), wherein optionally the mefloquine is formulated for oral administration, optionally in tablet or capsule form, optionally as 200 mg, 250 mg or 300 mg tablets;
   (f) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™ or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof,
   (g) an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
   and optionally the DDS system comprises: a polyether ester urethane comprising 65% D, L-lactide, 19% polyethylene glycol, and 16% glycolide interlinked with an aliphatic di-isocyanate, or comprises VISCOPRENE™,
   and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously,
   (h) a nicotinic antagonist, a dopamine agonist or a noncompetitive N-Methyl-D-aspartate (NMDA) antagonist, optionally amantadine, or GOCOVRI™, or SYMADINE™, or SYMMETREL™, optionally dosaged at between about 100 to 200 mg per dose, optionally formulated as tablets or capsules, or
   (i) a mitochondrial sensitizer, optionally proguanil or chlorguanide (or PALUDRINE™), or a malarial cytochrome bc1 complex inhibitor, optionally atovaquone (or MEPRON™), or a combination of proguanil and atovaquone (or MALARONE™), and/or
   (j) a drug combination or therapeutic regimen comprising any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i)
   in the manufacture of a medicament for preventing, or substantially preventing, decreasing the chances of having any adverse effects from, decreasing the severity of adverse effects from, or treating or ameliorating a viral infection or a microbial infection, or a protozoan, helminthiasis, insect and/or parasitic infection, in an individual in need thereof
2. The use of form 1, wherein the avermectin class drug comprises: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin.
3. The use of form 1 or form 2, wherein the drug or drug combination is administered to prevent or substantially prevent, or to treat or ameliorate, or decrease the severity of symptoms or pathology of:
   a viral infection, optionally a coronavirus, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection,
   a coronavirus infection, optionally a COVID-19 infection, optionally a COVID-19 variant infection, wherein optionally the COVID-19 variant is a delta or an omicron variant, or the coronavirus infection comprises a Middle East respiratory syndrome virus (MERS-CoV) infection;
   malaria that can be caused by a parasite of the genus Plasmodium (optionally P. vivax, P. falciparum, P. malariae, P. ovale, or P. knowlesi);
   dengue fever or dengue shock syndrome that can be caused by a virus of the Flaviviridae family or a dengue virus;
   hepatitis or hepatocellular carcinoma associated with viral hepatitis that can be caused by a virus of the Flaviviridae family or a virus of the genus Hepacivirus or Hepacivirus C virus or hepatitis C;
   filariasis, leprosy or streptocerciasis that can be caused by a parasite of the superfamily Filarioidea (optionally Brugia malayi, Brugia timori, Wuchereria bancrofti, Loa loa, Mansonella streptocerca, Mansonella ozzardi, or Mansonella perstans);
   leprosy that can be caused by a parasite of the genus Mycobacterium (optionally M leprae or M. lepromatosis);
   river blindness or onchocerciasis that can be caused by parasitic worms such as parasites of the genus Onchocerca (optionally O. volvulus);
   hookworm or roundworm infections that can be caused by parasites of the genus Ancylostoma (optionally A. duodenale or A. ceylanicum) or Necator (optionally N. americanus);
   trichuriasis or whipworm infection that can be caused by a parasite of the genus Trichuris (optionally T. trichiura); roundworm or an Ascaris infection that can be caused by Ascaris lumbricoides;
   mite-carried infections such as scabies that can be caused by the parasite of the genus Sarcoptes (optionally S. scabiei);
   infections such as typhus caused by lice or parasites of the order Phthiraptera (optionally Pediculus humanus capitis);
   enterobiasis that can be caused by pinworm or parasites of the genus Enterobius (optionally E. vermicularis); and/or
   pulicosis or infections cause by fleas or insects of the order Siphonaptera or of the genus Pulex (optionally P. irritans).
4. The use of any one of forms 1 to 3, wherein the loading dose of the avermectin class drug (optionally ivermectin) of between is about 15 to 150 mg/kg, or is about 18, 24, 30, 35, 40, 35, 50, 55, 60, 65, 70, 75, 80, 85, 90, 95, 100, 110 or 120 or more mg/kg.
5. The use of any one of the preceding forms, wherein the loading dosage is given once, or periodically, optionally every 2, 3, 4, 5, 6, 7, 8, 9, 10, 11, or 12 or more days.
6. The use of any one of the preceding forms, wherein the maintenance dosage of (a)(ii) is administered 1, 2, 3, 4, 5, 6, 7, 8, 9, 10, 11, 12, 13 or 14 days, or every 3 weeks or every month or every two months or longer after the first loading dosage.
7. The use of any one of the preceding forms, wherein the maintenance dosage of (a)(ii) is administered every 1, 2, 3, 4, 5, 6, 7, 8, 9, 10, 11, 12, 13 or 14 days, every 3 weeks, or monthly, over the 4 to 8 weeks, 6 to 10 weeks, 8 to 12 weeks, 10 to 20 weeks, 15 to 30 weeks or 20 to 52 weeks, or more, after the initial or loading dose is given.
8. The use of any one of the preceding forms, wherein: an antibiotic or anti-viral is administered with the loading dosage of the avermectin class drug (optionally ivermectin); zinc or a zinc salt or zinc chelate is administered with the loading dosage of the avermectin class drug (optionally ivermectin); or zinc or zinc chelate or a zinc salt and an antibiotic is administered with the loading dosage of the avermectin class drug (optionally ivermectin),
and optionally a drug combination, optionally formulated as one formulation (for example, as a tablet capsule) comprises: ivermectin, doxycycline and zinc chelate, or comprises: ivermectin 12 mg, doxycycline 100 mg and zinc chelate 25 mg.
9. The use of form 8, wherein the antibiotic comprises doxycycline, azithromycin or hydroxychloroquine (HCQ).
10. The use of any one of the preceding forms, wherein: an antibiotic or anti-viral is administered with the maintenance dose of the avermectin class drug (optionally ivermectin); zinc or a zinc salt or zinc chelate is administered with the maintenance dosage of the avermectin class drug (optionally ivermectin); or zinc or a zinc salt or zinc chelate and an antibiotic or anti-viral is administered with the maintenance dosage of the avermectin class drug (optionally ivermectin).
11. The use of form 10, wherein the antibiotic comprises doxycycline, azithromycin or hydroxychloroquine (HCQ).
12. The use of any one of the preceding forms, wherein an additional drug is or drugs, or therapy, is or are administered with the loading dose and/or the maintenance dose of the avermectin class drug (optionally ivermectin), or before the loading dose and/or the maintenance dose, or any time between administration of the loading dose and the maintenance dose.
13. The use of any one of the preceding forms, wherein an additional drug or therapy, is or are administered with the drug or drug combination of (a), (b), (c), (d), (e), (f), (g), (h) or (i), or any one or more of the following is administered with the drug combination or therapeutic regimen of any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i) of form 1:
   a thiazolide class drug, optionally nitazoxanide (or Alinia™, Nizonide™) or tizoxanide (or 2-Hydroxy-N-(5-nitro-2-thiazolyl)benzamide);
   molnupiravir, optionally co-administered with and/or formulated with an avermectin class drug (optionally ivermectin), an antibiotic (optionally doxycycline or azithromycin) and/or zinc, zinc salt or zinc chelate, or co-administered with and/or formulated with ivermectin, hydroxychloroquine, an antibiotic (optionally doxycycline or azithromycin) and/or zinc, zinc salt or zinc chelate;
   a mucolytic therapy or drug, optionally acetylcysteine, ambroxol, bromhexine (or BISOLVON™), carbocisteine, erdosteine, mecysteine or dornase alfa, or an expectorant, optionally guaifenesin;
   an H2 antagonist, or H2RA, or H2 blockers, or a compound, drug or formulation that decreases stomach acid production or decreases stomach pH, wherein optionally the compound, drug or formulation comprises famotidine (or PEPCID™), ranitidine (or ZANTAC™), nizatidine (or AXID™ or TAZAC™), roxatidine acetate, lafutidine, or cimetidine (or TAGAMET™), and optionally the famotidine is administered at a dosage of between about 10 to 60 mg per day, or between about 20 to 40 mg per day;
   a dendrimer, optionally astodrimer sodium (Starpharma, Melbourne, Australia);
   an antihistamine class drug such as azelastine, or ASTELIN™, OPTIVAR™, ALLERGODIL™, bepotastine (or TALION™, BEPREVE™) brompheniramine, fexofenadine or ALLEGRA™, pheniramine or AVIL™, or chlorpheniramine;
   a selective serotonin reuptake inhibitor (SSRI) class drug, optionally fluvoxamine, or LUVOX™, FAVERIN™, FLUVOXIN™;
   a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™ or ronifibrate, or simfibrate or CHOLESOLVIN™, or any combination thereof, clofazimine, or LAMPENE™, optionally dosaged at about 100 mg per day, or between about 50 mg and 150 mg per day, and optionally also including colchicine;
   clofazimine, or LAMPENE™, optionally dosaged at about 100 mg per day, or between about 50 mg and 150 mg per day, and chloroquine (or ARALEN™), chloroquine phosphate, chloroquine diphosphate and/or hydroxychloroquine (HCQ) (optionally, PLAQUENIL™), optionally also comprising zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally also including administration of zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine, a combination of clofazimine (optionally dosaged at about 100 mg or 150 mg per day, or between about 50 mg and 200 mg per day), fluvoxamine, and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), fluvoxamine and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally further comprising zinc, zinc salt or zinc chelate (optionally a zinc sulphate, acetate, gluconate or picolinate, or zinc oxide nanoparticles, optionally at a dosage of between about 1 mg to 250 mg), and optionally also including colchicine;
   hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);
   chloroquine (or ARALEN™), chloroquine phosphate, chloroquine diphosphate and/or hydroxychloroquine (HCQ) (optionally, PLAQUENIL™);
   a corticosteroid or glucocorticoid class drug such as such as ciclesonide (or Alvesco™, Omnaris™, Omniair™, Zetonna™ or Alvesco™), budesonide (optionally RHINOCORT™ or PULMICORT™), prednisolone (or ORAPRED™), methyl-prednisolone, prednisone (or DELTASONE™ or ORASONE™) or hydrocortisone (or CORTEF™), or a selective estrogen receptor modulator (SERM), or toremifene (or Fareston™), or clomifene or clomiphene (or CLOMID™, SEROPHENE™), wherein optionally the mode of administration for the corticosteroid or glucocorticoid class drug (optionally ciclesonide) is by inhalation (i.e., they are inhaled);
   a hydrocortisone or cortisol (optionally CORTEF™, SOLUCORTEF™), optionally hydrocortisone sodium succinate or hydrocortisone acetate or dexamethasome (optionally Dextenza™, Ozurdex™, Neofordex™),
      and optionally the corticosteroid or glucocorticoid class drug (for example budesonide or ciclesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,
      and optionally the corticosteroid or glucocorticoid class drug (optionally budesonide or ciclesonide) is formulated as a powder or for administration in an inhaler or by nasal spray, or for rectal administration,
      and optionally the corticosteroid or glucocorticoid class drug (for example, budesonide or ciclesonide) is administered together with or in combination with 10 mg to 80 mg, an antibiotic (optionally azithromycin or a tetracycline class drug,
      wherein optionally the tetracycline class drug comprises doxycycline, or DORYX™, DOXYHEXA™, DOXYLIN™), zinc, zinc salt or zinc chelate and/or a vitamin (optionally vitamin D or calcifediol, D2 (or ergocalciferol), D3 (or cholecalciferol), C, E, B12, B6);
   an anti-androgen drug, and optionally the anti-androgen drug is bicalutamide, optionally CASODEX™, or dutasteride (or AVODART™),
      and optionally the anti-androgen drug is a nonsteroidal anti-androgen (NSAA) or an androgen receptor (AR) antagonist, and optionally the NSAA or AR antagonist comprises proxalutamide (or its developmental name GT-0918) (Suzhou Kintor Pharmaceuticals, Inc., a subsidiary of Kintor Pharmaceutical Limited), or flutamide (or niftolide, or EULEXIN™), or bicalutamide (or CASODEX™) or enzalutamide (or XTANDI™),
      and optionally the anti-androgen drug comprises a 5α-reductase inhibitor, and optionally the 5α-reductase inhibitor comprises finasteride (or PROSCAR™, PROPECIA™, or FINIDE™),
      and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;
   an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2),
      and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin or a tetracycline class drug, wherein optionally the tetracycline class drug comprises doxycycline, or DORYX™, DOXYHEXA™, DOXYLIN™); chloroquine (or ARALEN™), chloroquine phosphate, chloroquine diphosphate and/or hydroxychloroquine (optionally, PLAQUENIL™); zinc, zinc salt or zinc chelate; remdesivir (optionally, GS-5734™, Gilead Sciences); oseltamivir (or TAMIFLU™); and/or, hydrocortisone; or, any combination thereof;
   a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or a tetracycline tetracycline class drug, and optionally the tetracycline class drug comprises doxycycline, or DORYX™, DOXYHEXA™, DOXYLIN™;
   a dendrimer, optionally astodrimer sodium (Starpharma, Melbourne, Australia);
   an antihistamine class drug such as azelastine, or ASTELIN™, OPTIVAR™, ALLERGODIL™, brompheniramine, fexofenadine or ALLEGRA™, pheniramine or AVIL™, or chlorpheniramine;
   a selective serotonin reuptake inhibitor (SSRI) class drug, optionally fluvoxamine, or LUVOX™, FAVERIN™, FLUVOXIN™;
   a nicotinic antagonist, a dopamine agonist or a noncompetitive N-Methyl-d-aspartic acid or N-Methyl-d-aspartate (NMDA) antagonist, wherein optionally the nicotinic antagonist, dopamine agonist or noncompetitive NMDA antagonist is 1-adamantylamine or amantadine, or GOCOVRI™ SYIVIADINE™, SYMMETREL™, optionally administered or dosaged at between about 50 mg to 150 mg, or about 100 mg, or 200 mg, per day for a period of between about 7 and 21 days, or about 14 days, and optionally the nicotinic antagonist, dopamine agonist or noncompetitive NMDA antagonist is also administered or formulated with an antibiotic (optionally azithromycin or doxycycline), ivermectin, hydroxychloroquine (optionally, PLAQUENIL™) and/or zinc, zinc salt or zinc chelate (optionally zinc sulfate, optionally at (50 mg daily), and optionally the amantadine is formulated or administered at 100 mg per day for the first two days of treatment, which optionally can then be elevated to 100 mg twice daily, optionally for the next 10 days;
   an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
      and optionally the DDS system comprises: a polyether ester urethane comprising 65% d,l-lactide, 19% polyethylene glycol, and 16% glycolide interlinked with an aliphatic di-isocyanate, or comprises VISCOPRENE™
      and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously;
   an immunosuppressive drug, wherein optionally the immunosuppressive drug comprises tocilizumab or atlizumab, or Actemra™, or RoActemra™, or a calcineurin inhibitor (CNI), wherein the CNI comprises ciclosporin (or cyclosporine or cyclosporin), or Neoral™, or Sandimmune™, or tacrolimus, or Protopic™, or Prograf™, and optionally the immunosuppressive drug is also administered or formulated with an antibiotic (optionally azithromycin or doxycycline), ivermectin, hydroxychloroquine (optionally, PLAQUENIL™) and/or zinc, zinc salt or zinc chelate (optionally zinc sulfate, optionally at (50 mg daily),
      and optionally the calcineurin inhibitor (CNI), wherein the CNI comprises ciclosporin (or cyclosporine or cyclosporin) is formulated combination of CNI (optionally cyclosporine) at a dose of 3 mg/kg (180 mg daily) together with 12 mg ivermectin once, and optionally also plus zinc 50 mg base and doxycycline 100 mg bid, optionally all for 10 days;
   a protein kinase inhibitor, wherein optionally the protein kinase inhibitor is a p38 mitogen-activated protein kinase inhibitor, or ralimetinib, and optionally the protein kinase inhibitor is also administered or formulated with an antibiotic (optionally azithromycin or doxycycline), ivermectin, hydroxychloroquine (optionally, PLAQUENIL™) and/or zinc or any zinc salt or zinc chelate (optionally zinc sulfate, optionally at (50 mg daily);
   an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally meplazumab), a cyclooxygenase (COX) (optionally, COX2) inhibitor, a glucocorticoid (optionally a synthetic glucocorticoid, hydrocortisone, dexamethasone (or DEXTENZA™, OZURDEX™, or NEOFORDEX™) or cortisol, or CORTEF™), plitidepsin or dehydrodidemnin B, or APLIDIN™, or a nonsteroidal anti-inflammatory drug (NSAID), wherein optionally the NSAID comprises indomethacin (or indomethacin) or INDOCID™ or INDOCIN™, or naproxen, or NAPROSYN™ or ALEVE™, or a cyclooxygenase inhibitor, or a COX-1 or an COX-2 inhibitor, or aspirin, or ibuprofen or ADVIL™ MOTRIN™ or NUROFEN™, or celecoxib or CELEBREX™, or parecoxib or DYNASTAT™, or etoricoxib or ARCOXIA™
      and optionally the anti-inflammatory therapy or anti-inflammatory therapy drug is also administered or formulated with an antibiotic (optionally azithromycin or doxycycline), ivermectin, hydroxychloroquine (optionally, PLAQUENIL™) and/or zinc or any zinc salt or zinc chelate (optionally zinc sulfate, optionally at (50 mg daily),
      and optionally opaganib, or YELIVA™, or opaganib, or YELIVA™ administered or formulated together with an oral and/or inhaled or aerosol chloroquine (or ARALEN™), chloroquine phosphate, chloroquine diphosphate and/or hydroxychloroquine (optionally, PLAQUENIL™),
      and optionally the opaganib or YELIVA™ is formulated or administered at a dosage of QD (once a day), bid (twice a day) or tid (three times a day) at a dosage of between about 100 to 600 mg per day or per dosage, or at about 100, 200, 300, 400, 500 or 600 mg per day or per dosage,
      and optionally the opaganib, or YELIVA™ is also administered or formulated with an antibiotic (optionally azithromycin or doxycycline), ivermectin (optionally at 12 mg ivermectin, optionally administered on days 1, 3, 6 and 8), hydroxychloroquine (optionally, PLAQUENIL™) and/or zinc or any zinc salt or zinc chelate (optionally zinc sulfate, optionally at (50 mg daily);
   a calcium channel blocker, or verapamil (or ISOPTIN™, CALAN™), or a voltage gated potassium (KCNH2) channel or a voltage gated calcium channel (CACNA2D2) blocker, or amiodarone (or CORDARONE™, NEXTERONE™);
   a suramin, or ANTRYPOL™, BAYER 305™, or GERMANIN™;
   a PPAR agonist, optionally fenofibrate, or TRICOR™, FENOBRAT™, FENOGLIDE™, or LIPOFEN™, and optionally the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™ or ronifibrate, or simfibrate or CHOLESOLVIN™, or any combination thereof, or a combination of fenofibrate and simvastatin, or CHOLIB™;
   a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™ Glenmark Pharmaceuticals),
      wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days,
      and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin),
      and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin) with an antibiotic, and optionally the antibiotic comprises azithromycin, minocycline, amoxicillin, niclosamide, nitazoxanide, hydroxychloroquine or doxycycline), and optionally the synthetic nucleoside analog or derivative, avermectin class drug, and antibiotic are administered together or as separate formulations, and optionally are administered every one, two, three, four or five weeks for between about one month and one year or more;
      and optionally molnuvpiravir, ivermectin and hydroxychloroquine are administered together or as separate formulations, and optionally are administered every one, two, three, four or five weeks for between about one month and one year or more;
      and optionally the synthetic nucleoside analog or derivative (optionally N4-hydroxycytidine, or the prodrug of N4-hydroxycytidine, optionally molnuvpiravir or favipiravir), and antibiotic (optionally doxycycline or hydroxychloroquine) is administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D),
      and optionally the synthetic nucleoside analog or derivative, or N4-hydroxycytidine, or the prodrug of N4-hydroxycytidine, optionally molnuvpiravir or favipiravir, is administered with an antibiotic (optionally the antibiotic comprises azithromycin, minocycline, amoxicillin, niclosamide, nitazoxanide, hydroxychloroquine or doxycycline), optionally also administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
         and optionally any of these combinations is administered very 2, 3, 4, 5, 6, 7, 8, 9 or 10 or more days for between about 1 month and one year or more;
   an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
   at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid);
   copper, optionally administered or formulated at a dosage of between about 1 to 200 mg per day, wherein optimally the copper is administered or formulated as cupric chloride and administered intravenously formulated at about 0.4 mg/ml;
   selenium, optionally administered as selenious acid formulated at about 65.4 mcg/ml (or μ/ml), and optionally the selenium is administered at a dosage of between about 50 to 100 optionally between about 60 to 100 μgm per day is administered to an adult, and only up to 60 μgm per day for pediatric patients;
   favipiravir (or T-705, avigan, or favilavir), optionally at 800 mg bid;
   zinc, zinc salt or zinc chelate (optionally a zinc sulphate, acetate, gluconate or picolinate) or zinc oxide nanoparticles, optionally at a dosage of between about 1 mg to 250 mg;
   colchicine, or COLCRYS™, MITIGARE™;
   at least one antibiotic or anti-viral (wherein optionally the antibiotic is doxycycline (optionally, DORYX™, DOXYHEXA™, DOXYLIN™) (optionally formulated or administered at a dosage of between about 25 mg to 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
   at least one anti-viral drug or medication, or anti-microbial drug, or palliative agent or drug, wherein optionally the anti-viral drug or medication, or anti-microbial drug, is or comprises efavirenz (for example, SUSTIVA™), tenofovir (optionally tenofovir alafenamide or tenofovir disoproxil, or VIREAD™), emtricitabine and tenofovir, nevirapine (or the combination efavirenz with emtricitabine and tenofovir, or ATRIPLA™), amprenavir (for example, AGENERASE™), nelfinavir (for example, VIRACEPT™) and/or remdesivir (for example, GS-5734™, Gilead Sciences), a viral RNA-dependent RNA polymerase inhibitor, optionally favipiravir (optionally AVIGAN™) or sofosbuvir (optionally SOVALDI™, SOFORAL™); or, an adenosine analog (optionally galidesivir, optionally BCX4430, IMMUCILLIN-A™),
      and optionally the anti-viral drug or medication is or comprises an anti-retroviral drug or drug combination, and optionally the anti-retroviral drug or drug combination comprises: darunavir and cobicistat (for example, REZOLSTA™ or PREZCOBIX™); atazanavir (or REYATAZ™) and cobicistat (or EVOTAZ™); abacavir, lamivudine and dolutegravir (TRIUMEQ™); tenofovir (or tenofovir disoproxil or tenofovir disoproxil, or VIREAD™, or emtricitabine) and elvitegravir and cobicistat (for example, STRIBILD™); tenofovir (or disoproxil or emtricitabine) and elvitegravir and cobicistat (COMPLERA™ or EVIPLERA™); efavirenz (optionally, SUSTIVA™), emtricitabine and tenofovir (or ATRIPLA™); lamivudine, nevirapine and stavudine (for example, TRIOMUNE™); atazanavir (or REYATAZ™) and cobicistat (for example, EVOTAZ™); lamivudine and raltegravir (for example, DUTREBIS™); lamivudine and dolutegravir (or DOVATO™); doravirine, lamivudine and tenofovir (for example, DELSTRIGO™); or lamivudine, zidovudine and nevirapine (for example, CUOVIR-NTH), and optionally the anti-viral drug or drug combination comprises daclatasvir (optionally DAKLINZA™);
   a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
   a viral, or a coronavirus or a COVID-19, protease inhibitor, wherein optionally the protease inhibitor comprises: ASC09 (CAS registry no. 1000287-05-7) (Janssen Research and Development, LLC), ritonavir (optionally NORVIR™) or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir, cobicistat or darunavir and cobicistat, or PF-07321332, or nirmatrelvir, or the combination of nirmatrelvir and ritonavir, or PAXLOVID™, PF-07304814 or PF-008335231 (Pfizer), or remdesivir (for example, GS-5734™, Gilead Sciences) or ritonavir (optionally NORVIR™) in combination with the PF-07321332, or nirmatrelvir, or the combination of nirmatrelvir and ritonavir, or PAXLOVID™, PF-07304814 or PF-008335231 (Pfizer) optionally as an oral formulation, optionally as a tablet, geltab or capsule,

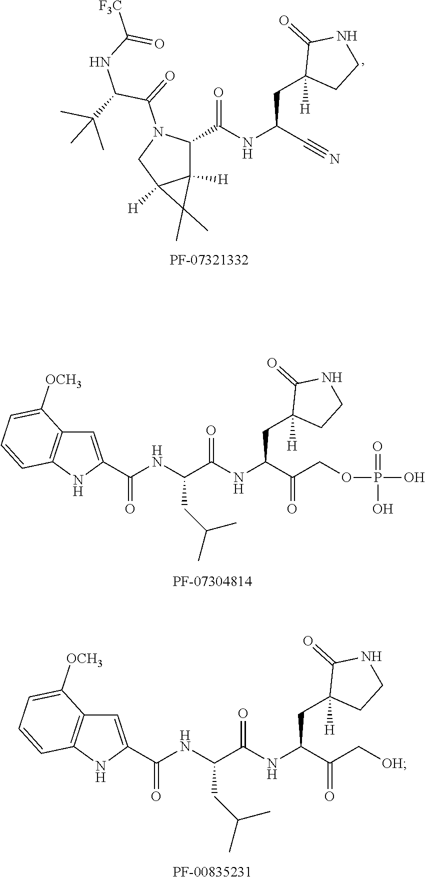 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C ; COc1cccc2[nH]c(C(=O)N[C@@H](CC(C)C)C(=O)N[C@@H](C[C@@H]3CCNC3=O)C(=O)COP(=O)(O)O)cc12 ; COc1cccc2[nH]c(C(=O)N[C@@H](CC(C)C)C(=O)N[C@@H](C[C@@H]3CCNC3=O)C(=O)CO)cc12]

a thiazolide class drug, optionally nitazoxanide (or ALINIA™, NIZONIDE™) or tizoxanide (or 2-Hydroxy-N-(5-nitro-2-thiazolyl)benzamide);
a blood clot inhibiting drug such as aspirin, warfarin (or COUMADIN™) or rivaroxaban (or XARELTO™);
lopinavir, ritonavir (optionally NORVIR™) or the combination of lopinavir and ritonavir (or KALETRA™, ALTERA™, ALUVIA™, KALMELTREX, or LOPIIVIUNE™), opaganib (or YELIVA™), oseltamivir (or TAMIFLU™), and/or zanamivir (or RELENZA™);
an inhibitor or S-phase kinase-associated protein 2 (SKP2), or dioscin, or niclosamide, or NICLOCIDE™, FENASAL™, or PHENASAL™;
a tyrosine kinase inhibitor (TKi), wherein the TKi comprises: masitinib (or MASIVET™, or KINAVET™); or imatinib (or GLEEVEC™, GLIVEC™); or gefitinib (or IRESSA™), or erlotinib (or TARCEVA™), or dasatinib (or SPRYCEL™, DASANIX™);
ribavirin or tribavirin (or COPEGUS™, REBETOL™, or VIRAZOLE™), interferon beta 1b, or a combination of ribavirin and interferon beta, or a combination of lopinavir and ritonavir (optionally NORVIR™) and interferon-beta-1b;
a nucleoside analog reverse-transcriptase inhibitor (NRTI) (optionally abacavir, or ZIAGEN™), acyclovir (or ZOVIRAX™), optionally ACICLOVIR™), adefovir (optionally HEPSERA™), amantadine (optionally GOCOVRI™, SYMADINE™, SYMMETREL™), rintatolimod (or AMPLIGEN™), amprenavir (optionally, AGENERASE™), aprepitant (or EMEND™), umifenovir (or ARBIDOL™), atazanavir (or REYATAZ™), tenofovir or tenofovir disoproxil (or VIREAD™), a combination of efavirenz and emtricitabine and tenofovir (or ATRIPLA™), balavir, baloxavir marboxil (XOFLUZA™), bepotastine (or TALION™, BEPREVE™), bevirimat, bictegravir, biktarvy, brilacidin, cidofovir, caspofungin, lamivudine and zidovudine (optionally, COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir (or VIRACEPT™), nevirapine, nexavir, nitazoxanide, norvir, a nucleoside analogue (optionally brincidofovir, didanosine, favipiravir (also known as T-705, AVIGAN™, or favilavir, Toyama Chemical, Fujifilm, Japan), vidarabine, galidesivir (optionally, BCX4430, IMMUCILLIN-ATM), remdesivir (optionally, GS-5734TH, Gilead Sciences), cytarabine, gemcitabine, emtricitabine, lamivudine, zalcitabine, entecavir, stavudine, telbivudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir (optionally NORVIR™), saquinavir, sofosbuvir, stavudine, telaprevir, tegobuv, tenofovir (optionally tenofovir alafenamide, tenofovir disoproxil or VIREAD™), tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™) or any combination thereof;
fenofibrate, or TRICOR™, FENOBRAT™, FENOGLIDE™, or LIPOFEN™, or a combination of fenofibrate and simvastatin, or CHOLIB™;
suramin, or ANTRYPOL™, BAYER 305™, or GERMANIN™;
a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™ Glenmark Pharmaceuticals),
   wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 5 to 15 days, or for about 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days,
   and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin),
   and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin) and an antibiotic, and optionally the antibiotic comprises azithromycin, minocycline, amoxicillin, niclosamide, nitazoxanide, hydroxychloroquine or doxycycline), and optionally the synthetic nucleoside analog or derivative, avermectin class drug, and antibiotic are administered together or as separate formulations, and optionally are administered every one, two, three, four or five weeks for between about one month and one year or more;
   and optionally molnuvpiravir, ivermectin and hydroxychloroquine are administered together or as separate formulations, and optionally are administered every one, two, three, four or five weeks for between about one month and one year or more;
   and optionally the synthetic nucleoside analog or derivative (optionally N4-hydroxycytidine, or the prodrug of N4-hydroxycytidine, optionally molnuvpiravir or favipiravir), and antibiotic (optionally doxycycline or hydroxychloroquine) is administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D),
   and optionally the synthetic nucleoside analog or derivative, or N4-hydroxycytidine, or the prodrug of N4-hydroxycytidine, optionally molnuvpiravir or favipiravir, is administered with at least two antibiotics (optionally the at least two antibiotics comprise azithromycin, minocycline, amoxicillin, niclosamide, nitazoxanide, hydroxychloroquine and/or doxycycline), and a zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
an antibody or antibody or vaccine therapy for treating, preventing or ameliorating a microbial or a viral infection (optionally a coronavirus infection, optionally a COVID-19 infection) or a microbial infection (optionally a protozoan, helminthiasis, insect and/or parasitic infection), and optionally the antibody comprises a monoclonal antibody, and optionally the monoclonal antibody comprises sotrovimab (GlaxoSmithKline and Vir Biotechnology), or casirivimab, imdevimab or casirivimab and imdevimab (REGEN-COV™) (Regeneron), or bamlanivimab oretesevimab or bamlanivimab and etesevimab (Junshi Biosciences), or tocilizumab or ACTEMRA™ or ROACTEMRA™ (Hoffmann-La Roche),
   and optionally the antibody or vaccine therapy comprises tozinameran or COMIRNATY™ (Pfizer), or elasomeran or SPIKEVAX™ (Moderna), or SPUTNIK V™ or Gam-COVID-Vac (Gamaleya Research Institute), or AZD1222 or COVISHIELD™ or VAXZEVRIA™ (Oxford-AstraZeneca),
   and optionally the antibody or antibody therapy comprises or is contained in a convalescent sera or plasma,
wherein optionally any of these combinations is administered very 2, 3, 4, 5, 6, 7, 8, 9 or 10 or more days for between about 1 month and one year or more; and/or
or any combination thereof.
14. The use of any one of the preceding forms, wherein a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) are administered:
(a) once a month; or
(b) for the first four, five, six or seven days of treatment an avermectin class drug (optionally ivermectin) is given at about 24 mg per day or between about 20 to 30 mg per day, doxycycline or azithromycin is given at about 100 mg per day or between about 50 and 150 mg per day, clofazimine is given about 100 mg per day or between about 50 and 150 mg per day, and zinc, zinc salt or zinc chelate (optionally a zinc sulphate, acetate, gluconate or picolinate, or zinc oxide nanoparticles) is administered at a dosage of between about 25 mg to 100 mg per day, or about 50 mg per day),
and after this initial first four, five, six or seven days of treatment a once a month maintenance regimen of an avermectin class drug (optionally ivermectin) dosaged at between about 60 to 80 mg, or about 60 mg, clofazimine dosaged at about 100 mg or between about 50 to 150 mg, doxycycline or azithromycin dosaged at about 100 mg or between about 50 to 150 mg, and zinc, zinc salt or zinc chelate (optionally a zinc sulphate, acetate, gluconate or picolinate, or zinc oxide nanoparticles) is administered at a dosage of between about 25 mg to 100 mg per day, or about 50 mg per day) is given,
and optionally the avermectin class drug (optionally ivermectin), clofazimine, doxycycline or azithromycin and zinc are formulated and administered in separate dosage units (optionally geltabs, tablets, capsules), or the avermectin class drug (optionally ivermectin), clofazimine, doxycycline or azithromycin and zinc, zinc salt or zinc chelate are formulated and administered in one unit dosage (optionally all in one a geltab, tablet, capsule).
15. The use of any one of the preceding forms, wherein the individual in need thereof suffers from long term effects, or chronic effects or symptoms, of a viral infection, or the individual in need thereof has not fully recovered from the viral infection weeks or even months after first experiencing symptoms, or the individual in need thereof experiences continuous symptoms for weeks or months after being first diagnosed or treated with the viral infection, or the individual in need thereof feels better for weeks, then relapses with old or new symptoms,
and optionally the medication or the drug combination is administered to prevent a so-called “long-hauler” syndrome, or to treat or prevent continuous symptoms for weeks or months, or to prevent or treat relapsing with old or new symptoms,
wherein optionally the viral infection is a COVID-19 infection.
16. The use of any one of the preceding forms, wherein the synthetic nucleoside analog or derivative, or N4-hydroxycytidine, or the prodrug of N4-hydroxycytidine, optionally molnuvpiravir or favipiravir, is administered with an antibiotic (optionally the antibiotic comprises azithromycin, minocycline, amoxicillin, niclosamide, nitazoxanide, hydroxychloroquine or doxycycline), optionally also administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
and optionally the synthetic nucleoside analog or derivative, or N4-hydroxycytidine, or the prodrug of N4-hydroxycytidine, optionally molnuvpiravir or favipiravir, is administered with azithromycin and/or hydroxychloroquine and/or doxycycline with an avermectin class drug optionally comprising: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
and optionally the synthetic nucleoside analog or derivative, or N4-hydroxycytidine, or the prodrug of N4-hydroxycytidine, optionally molnuvpiravir or favipiravir, is administered with opinavir, ritonavir (optionally NORVIR™), or the combination lopinavir and ritonavir (or KALETRA™, ALTERA™, ALUVIA™, KALMELTREX or LOPIMUNE™), opaganib (or YELIVA™), oseltamivir (or TAMIFLU™), and/or zanamivir (or RELENZA′).

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### BRIEF DESCRIPTION OF DRAWINGS

The drawings set forth herein are illustrative of exemplary embodiments provided herein and are not meant to limit the scope of the invention as encompassed by the claims.

FIG. 1 illustrates the four pillars of infection of the COVID-19 pandemic, include the stopping of the spread by contagion control as achieved by distancing and mask wearing, as represented by the first pillar (Pillar I). The second pillar (Pillar II) is early home treatment or ambulatory treatment as soon as the patient has acquired the infection to prevent them from developing more and more severe disease with low oxygen, breathlessness, muscle pains, cough and finally fever. This treatment is the crucial treatment that could turn a pandemic around if given early enough to enough patients simultaneously or serially. It is generally given on a daily basis for at least 5 days, but used over 10 days it has a near 100% effectiveness. The third pillar (Pillar III) of the treatment of pandemic response is the late-stage or treatment in hospital. Here, the patients develop initially an early cytokine release, going through to greater release, ultimately resulting in what is called a severe cytokine storm. The released active molecules from the infected tissues, especially in the lungs, and create inflammation and/or pneumonia and finally fibrosis of the lungs with oxygen tensions falling below SPO₂ of less than 90 on pulse oximetry. Ultimately, multiple vessels can thrombose and the patient can suffer from thromboembolic disease. The thromboses can also cause stroke and death of tissues wherever the clots occur. As the final or fourth pillar (Pillar IV) of managing infectious disease in the Coronavirus pandemic, it is mass vaccination to attain ‘herd immunity’ where the majority of the population is immune to the Covid-19 infection. However, because vaccination in some people does not last for a prolonged period of time, a common finding with RNA viruses, it may need to be repeated and the signal for repeating the vaccination is at this stage unclear since titers of neutralizing antibodies may need to be measured.

Like reference symbols in the various drawings indicate like elements.

### DESCRIPTION OF EMBODIMENTS

In alternative embodiments, provided are pharmaceutical compositions and therapeutic combinations of drugs and methods for using them for preventing or ameliorating, or decreasing the chances of having any adverse effects from, decreasing the severity of adverse effects from, or treating or ameliorating a viral infection such as a coronavirus infection or a microbial infection including a protozoan, helminthiasis, insect and/or parasitic infection such as: malaria that can be caused by a parasite of the genus Plasmodium; dengue; filariasis, leprosy or streptocerciasis that can be caused by a parasite of the superfamily Filarioidea; leprosy that can be caused by a parasite of the genus Mycobacterium; river blindness or onchocerciasis that can be caused by parasitic worms such as parasites of the genus Onchocerca; hookworm or roundworm infections that can be caused by parasites of the genus Ancylostoma or Necator; trichuriasis or whipworm infection that can be caused by a parasite of the genus Trichuris; roundworm or an Ascaris infections; mite-carried infections such as scabies; infections such as typhus caused by lice or parasites of the order Phthiraptera; enterobiasis that can be caused by pinworm or parasites of the genus Enterobius; pulicosis or infections cause by fleas or insects of the order Siphonaptera or of the genus Pulex, and other infections and infestations. In alternative embodiments, provided are manufactured kits and specialized tablets and capsules, and slow-release technology for delivering pharmaceutical compositions and therapeutic combinations of drugs as provided herein and practicing methods as provided herein.

In alternative embodiments, combinations or cocktails of drugs are provided to be administered intravenously, orally, by inhalation, by suppository or parenterally or subcutaneously to treat and/or prevent a viral infection such as a coronavirus infection (such as a COVID-19 infection) or a microbial infection including a protozoan, helminthiasis, insect and/or parasitic infection as provided herein. In alternative embodiments, combinations or cocktails of drugs as provided herein are administered in higher doses than usual clinical state of the art, or what is considered “best practice”, doses to prolong the drug or drug combination's half-life and increase the ‘area under the curve’ (AUC) resulting from specialized dosing.

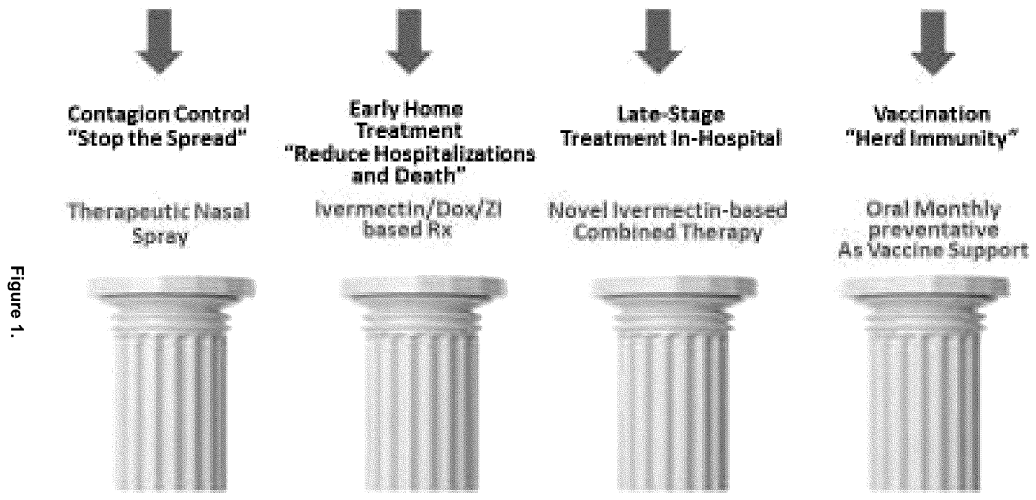

The approach to the preventative treatment disclosed herein is summarized in FIG. 1, which illustrates the four pillars of coronavirus infection management response. The fourth pillar, described as a ‘vaccine support’ is how oral preventative treatment as provided herein is combined with a vaccine or is used to replace a vaccine if the approved vaccines did not work as well as expected.

The underlying characteristics of ivermectin pharmacokinetic behavior include absorption which peaks in between about 2 to 5 hours, half-life in the plasma which may be between about 12 to 22 hours or about 18 hours (h), and then excretion mostly in the feces. Ivermectin can be metabolized in the liver to a great extent but there is another important component—that of tissue storage, particularly fat tissue storage. In both animals and humans, ivermectin is highly lipophilic and binds to or is absorbed by adipose tissue or fat; ivermectin distributes in the body with a volume of distribution (Vd) of 3.1 to 3.5 l/kg. Hence, once administered, orally repeatedly ivermectin accumulates in adipose tissue and it is slowly eluted from the fat tissues. Ivermectin may persist in the adipose tissue for many weeks and depending on the drug level in the fat may be slowly passed into the plasma for some weeks.

In the ivermectin, doxycycline or azithromycin and zinc therapeutic combination or composition for treatment of patients with Covid-19 in the ambulatory or early infectious phase (see FIG. 1, Pillar II), the two ionophores ivermectin and doxycycline are present to shepherd the zinc into the cells where the zinc acts to prevent the replication of Coronavirus RNA. The ivermectin and doxycycline are both extraneous molecules not normally present in the tissues, whereas zinc is normally present in circulation and tissues. Zinc may need to be supplemented in therapy, especially in those who are deficient in zinc, a common condition in the elderly. Ivermectin in itself without the use of doxycycline or azithromycin can be effective because of the zinc naturally occurring in the tissues to bring into the cells to inhibit the growth of Coronavirus.

In alternative embodiments, provided are methods for reducing the need for frequent administration of an avermectin class drug (optionally ivermectin) dosaging in a preventative therapy comprising either increasing the dose of the avermectin class drug (higher AUC), using it more frequently, or administering an avermectin class drug slow-release medication that would permit less frequent usage of the preventative medication.

In alternative embodiments, methods that have been employed in creating a slow-release of an avermectin class drug such as ivermectin alone in the treatment of malaria can be used. Similar but improved technology as provided herein is used when combining the avermectin class drug (optionally ivermectin) with the other therapeutic components or drugs (such as zinc and/or an antibiotic or anti-viral) to result in a treatment that can be used less frequently by the patient, but is able to completely protect the individuals from acquiring the clinical disease, for example, the disease caused by the Coronavirus, or the COVID-19 infection.

In the fourth (IV) pillar of therapy as illustrated in FIG. 1, the oral, intermittent preventative therapy as provided herein is used as a support therapy for a vaccine. In alternative embodiments, provided is an avermectin class drug (optionally ivermectin)-based prevention therapy which taken on an approximately monthly basis, an individual could depend on both the ivermectin-based prophylactic therapy, as well as the vaccine to prevent acquisition of the infection by individuals exposed to it. Given the vaccine is expected to be less active in the elderly and immunocompromised, prophylactic therapy as provided herein can be more important in this cohort than vaccination.

In alternative embodiments, drugs combinations and methods as provided herein are used for the treatment and prevention of infections, for example, Covid-19 infections, in patients at any one of the four (IV) pillar stages as illustrated in FIG. 1, but are particularly effective in stopping the spread of the contagion and as support for the vaccine.

In alternative embodiments, drug formulations and drugs combinations and methods as provided herein are used for the prevention of infection, for example, for use in individuals who are not infected. In alternative embodiments, drug formulations and drugs combinations are given weekly, second weekly (or every other week), monthly, or less frequently if the plasma levels of ivermectin remain high, or more frequently if the plasma levels of ivermectin remain low.

In alternative embodiments, to maintain high levels of an avermectin class drug (optionally ivermectin), for example, high ivermectin levels, or to store the avermectin class drug (optionally ivermectin) in the body's adipose tissue, a loading dose is used with ongoing intermittent treatment(s) that maintain the fatty tissue as a reservoir, slowly eluting the avermectin class drug (optionally ivermectin) over time, for example, over the next 4 to 8 weeks, or 6 to 10 week, or 8 to 12 weeks, or more, after the initial or loading dose is given.

In alternative embodiments, a loading dosage of an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is about 30 mg/kg (or 30 mg per 2.2 pounds (lb)) or about 1800 mg in a 60 kg (about 132 lb) person. In alternative embodiments, a loading dosage of ivermectin is between about 30 to 60 mg/kg or is between about 1800 mg to 3600 mg in a 60 kg (about 132 lb) person.

In alternative embodiments, a maintenance dosage of the avermectin class drug (for example, ivermectin) is between about 20 mcg/kg (μ/kg) to 5000 mcg/kg (μ/kg) or between about 200 to 2000 mcg/kg (μ/kg) dose, where 200 mcg/kg is equivalent to 12 mg in a 60 kg adult, and 2000 mcg/kg is 120 mg which is 7% of the LD₅₀. The accepted therapeutic dose of ivermectin for parasite treatment is 200 mcg per kg of body weight; however, studies have described use of more than 200 mcg/kg (12 mg) and 2000 mcg/kg (120 mg) and above this without any increase in ivermectin toxicity.

In alternative embodiments, provided is a drug or therapeutic combination comprising an avermectin class drug such as ivermectin, an antibiotic (for example, doxycycline or azithromycin) or anti-viral and zinc; for example, the drug or therapeutic combination comprises: ivermectin about 120 mg, doxycycline about 200 mg and about 50 mg of zinc (such as zinc picolinate acid, or a zinc sulphate, acetate, gluconate or picolinate salt) or other zinc salts or zinc-comprising compounds. In alternative embodiments, provided is a drug or therapeutic combination comprising between about 18 to 150 mg ivermectin, about 200 to 500 mg doxycycline or azithromycin and about 50 to 100 mg of zinc (such as zinc picolinate acid,) or a zinc or other zinc salts or zinc-comprising compounds.

In alternative embodiments, with the first or loading dosage, or after the first (i.e., with the second), third, fourth, fifth or sixth or more drug or drug combination administration, injected, oral, inhaled or aerosol formulations or versions of chloroquine (or ARALEN™), chloroquine phosphate, chloroquine diphosphate and/or hydroxychloroquine (HCQ) (optionally, PLAQUENIL™) is co-administered with an exemplary drug combination or formulation, because for example the 1/2 life of HCQ can be between about 50 to 70 days.

In alternative embodiments, with the first or loading dosage, or after the first (i.e., with the second), third, fourth, fifth or sixth or more drug or drug combination administration, particularly if zinc is included in the administered drug combination, copper, a vitamin such as vitamin D, vitamin C, vitamin E or vitamin A, and/or selenium is also administered, for example, in the same formulation as the avermectin class drug such as ivermectin, the antibiotic (for example, doxycycline or azithromycin) or anti-viral and/or the zinc, or the copper, the vitamin and/or the selenium is administered as a separate formulation.

In alternative embodiments, the copper is administered or formulated at a dosage of between about 1 to 200 mg per day. In alternative embodiments, the copper is administered or formulated as cupric chloride and administered intravenously formulated at about 0.4 mg/ml.

In alternative embodiments, the selenium is administered as selenious acid formulated at about 65.4 mcg/ml (or μ/ml), and optionally the selenium is administered at a dosage of between about 50 to 100 optionally between about 60 to 100 μgm per day is administered to an adult, and only up to 60 μgm per day for pediatric patients.

In alternative embodiments, this exemplary drug or therapeutic combination is administered (for example, prophylactically to an uninfected individual) as the loading dosage or dosages, and then this drug or therapeutic combination is administered weekly or monthly or every second month (or every two or three or more months), or every other week, or every third week. In alternative embodiments, this exemplary drug or therapeutic combination is administered (for example, prophylactically to an uninfected individual) on a monthly basis to slowly build up the fat accumulation of the ivermectin in the body.

Because the ivermectin reaches peak plasma levels by around 4 hours, and once loaded into body stores such as fat it then can remain in the blood stream for several weeks, it would be able to not only treat and eradicate, but also prevent infection when the patient is exposed to the pathogen or parasite, for example, to prevent infection when the individual (or patient) is exposed to someone who is already infected.

The vaccines do not work until an individual has developed immunity with rising IgM then IgG antibodies which may take around 14 to 28 days. Then, depending on the vaccine's quality, the neutralizing antibodies may remain in circulation for 3, 4, 5, 6 or more months. With an RNA virus, the immunization has problems because the persistence of the neutralizing antibodies can be short-lived, though in some patients the persistence of neutralizing antibodies is longer if the individual is re-immunized. Thus, in alternative embodiments, drug or therapeutic combinations as provided herein are used as a ‘vaccine support’ for a ‘weak’ vaccine. In alternative embodiments, drug or therapeutic combinations as provided herein are used prophylactically, and can be administered on a monthly basis, for example. In alternative embodiments, drug or therapeutic combinations as provided herein are used as an oral preventative or prophylactic therapy, for example, while a vaccine provides a low-level supplementary preventive measure to keep individuals free of COVID-19 infection.

When the avermectin class drug (optionally ivermectin) is used frequently, for example, on a daily or weekly basis for up to 8 weeks, for example, the drug accumulates in adipose tissue. After the 8 weeks the adipose tissue will have accumulated enough of the avermectin class drug (optionally ivermectin) for its protective effect to last for another 4 months by leaching out of the adipose tissue and into the plasma.

The zinc, which may be a necessary additive, becomes available from either the human tissue and/or from dietary and/or oral zinc, and so the ongoing available avermectin class drug (optionally ivermectin) can work through normally present tissue zinc, will be able to maintain protection for at least 4 weeks.

In alternative embodiments, an initial (or loading) treatment is the drug combination comprising ivermectin 120 mg, doxycycline or azithromycin 200 mg and 50 mg zinc Picolinate or equivalent, or a zinc salt.

In alternative embodiments, in order to load the adipose tissue, the treatment (methods as provided herein) are adapted to have another single dose of this exemplary drug combination (i.e., of an avermectin class drug (optionally ivermectin) 120 mg, doxycycline or azithromycin 200 mg and 50 mg zinc Picolinate or equivalent, or a zinc salt). In alternative embodiments, this exemplary drug combination is administered two weeks later, or at weeks 1, 2, 3 and 4, and from then on, on a monthly basis, because the avermectin class drug (optionally ivermectin) will be stored and will be leaching out of the adipose tissue.

In alternative embodiments, drug or therapeutic combinations as provided herein are formulated in liposomes, micelles such as oleic acid comprising micelles or liposomes, or other microparticles or nanoparticles, and in some embodiments only the avermectin class drug (optionally ivermectin) component of the drug or therapeutic combinations as provided herein is formulated in liposomes or in micelles such as oleic acid comprising micelle or liposomes.

In alternative embodiments, liposomes, micelles and/or other microparticles or nanoparticles are used to reduce drug metabolism and enhance the release of large quantities of the active avermectin class drug (optionally ivermectin) over a long period of time to the target site by altering its pharmacokinetics.

In alternative embodiments, routes of administration of exemplary drug or therapeutic combinations as provided herein comprise oral, topical, intravenous (IV), intramuscular (IM), inhalation (for example, by aerosol or nebulizer) and/or subcutaneous routes.

In alternative embodiments, oral administration is used because injection of a patient may not be acceptable for routine use in large populations. However, oral administration of avermectin class drug (optionally ivermectin) may have low bioavailability due to binding of the drug with the organic contents in the gut. Hence, a large percentage of the orally administered avermectin class drug (optionally ivermectin) may be excreted in the feces.

In alternative embodiments, to address this possible problem, oral formulations comprising exemplary drug or therapeutic combinations as provided herein are formulated in or for: ingestion with food or liquid (such as for example, beer or a stabilized aqueous formulation); administration with or using an osmotic pump; controlled release capsules; silicone carriers; zein or chitosan microspheres; biodegradable microparticle drug delivery system; and/or, biodegradable subcutaneous implants. Ivermectin absorption can also be increased 2 to 3 fold when ingested with a meal, particularly a fatty meal, or with some drinks such as beer.

In alternative embodiments, exemplary drug or therapeutic combinations as provided herein are formulated in lipid nanocapsules and nanoparticles.

In alternative embodiments, exemplary drug or therapeutic combinations as provided herein are formulated in oral delivery vehicles (for example, capsules) capable of prolonged drug delivery; where the exemplary oral delivery vehicle or formulation is a capsule, pill, tablet and the like which after ingestion resides in the stomach cavity, and its passage out of the stomach is delayed, and while in the stomach the oral delivery vehicle or formulation delivers the exemplary drug or therapeutic combinations as provided herein slowly to the small bowel for absorption.

In alternative embodiments, such oral delivery vehicles carry exemplary drug or therapeutic combinations as provided herein which can comprise large loads of therapeutic agents as described herein, and can provide control of release and maintain stability of the therapeutic agent at a low pH gastric environment for extended duration.

In alternative embodiments, such oral delivery vehicles can be located in the stomach without increasing potential for obstruction through the pylorus and avoid downstream intestinal obstruction, especially at the ileocecal valve. Such an object to be in the stomach has to be greater than 2 cm in diameter in research on malarial treatment, a tetrahedron has the critical size and shape and the optimal geometry; alternatively, a stellate-type encapsulated product is used for slow release. In alternative embodiments, four or more arms point out from a central hub.

In alternative embodiments, tetrahedrons or stellate-type encapsulated products as provided and used herein are built from degradable or dissolvable elements within the formulation which remain stable in acidic environments but dissolve in near-neutral pH, down in the small bowel. In alternative embodiments, polycaprolactone (PCL) or poly(ε-caprolactone) (or poly(epsilon-caprolactone)) or equivalents are used because these compounds are rigid enough as a drug release vehicle after being formed as a matrix because of its biocompatibility and low temperature melting process; it has been used successfully to deliver drugs in animal studies. In alternative embodiments, PCL equivalents comprise poly(ethylene oxide)-b-poly(alpha-cholesteryl carboxylate-epsilon-caprolactone), poly(alpha-benzylcarboxylate-epsilon-caprolactone) (PBCL) and/or poly(alpha-cholesteryl carboxylate-epsilon-caprolactone) (PChCL), which are used to make drug-encapsulating formulations or delivery vehicles as provided herein, for example, to make tetrahedrons or stellate-type encapsulated products. In alternative embodiments, PCL equivalents comprise biodegradable amphiphilic polyurethane block copolymers with hyperbranched structure (for example, where these copolymers are synthesized by copolymerizing poly(ε-caprolactone) (PCL) and poly(ethylene glycol) (PEG) together with glycerol).

In alternative embodiments, exemplary oral delivery vehicles have a size greater than about 2 cm in cross-sectional dimension; and when used in humans daily food intake will not interfere with the gastric residence of the drug delivery system, nor will cause obstruction at the pyloric sphincter. In alternative embodiments, exemplary oral delivery vehicles provided or used herein have the shape a tetrahedron or are star shape forms to for example reduce unanticipated drug release or dumping and to facilitate incorporation of various drugs such as the doxycycline or azithromycin and the zinc into a single exemplary delivery product as provided herein.

In alternative embodiments, exemplary oral delivery vehicles are shaped as a compressed spring which opens to prevent exit from the stomach. In alternative embodiments, exemplary oral delivery vehicles comprise radiopaque components which allow gastric residence to be evaluated by serial X-Rays if necessary, or by an electronic method of being picked up in a detection device worn around the abdomen. When tested in animals, such a product does not cause any gastric mucosal surface injury or ulceration. Such a device for gastric residence does not simply sit in the pylorus, but does move around the fundus and body of the stomach, and even fibrous foods did not cause any potential for obstruction in animal studies. Such a product can continue delivering the medications for over 40 days, for example, with an avermectin class drug (optionally ivermectin) delivered as a powdered product into a PCL or PCL equivalent, and optionally in a separate arm of an exemplary delivery product different drugs are inserted to ensure that a drug combination as provided herein, for example, an avermectin class drug (optionally ivermectin), doxycycline or azithromycin and zinc combination, can be delivered slowly over several weeks.

In one exemplary applications (compositions or methods) as provided herein, three or more major features are used to deliver the drugs for a prolonged period. First, an increased dose of an avermectin class drug (optionally ivermectin) is used or delivered. Second, initial frequent dosing or one loading dose is used to establish an adipose tissue (fat) reservoir; and this can be every few days or weekly followed by intermittently dosed every 1, 2, 3 or 4 or more weeks to maintain the avermectin class drug (optionally ivermectin) reservoir. Third, technology that delivers a slow release of a drug combination as provided herein, for example, an avermectin class drug (optionally ivermectin), doxycycline or azithromycin and/or zinc combination, over many days or weeks.

In alternative embodiments, additional or alternative group or groups of medications are included in a drug combination as provided herein or used in a method as provided herein, including use in a timed-release system as provided herein.

In alternative embodiments, optionally to increase efficacy of the avermectin class drug (optionally ivermectin) high doses per body weight, multiple dose regimens, and/or slow release formulations are used to increase the area under the curve and hence the efficacy and lethality for an infection such as a coronavirus infection; and co-therapies with various compounds also can be used. For example, as an alternative therapy to the avermectin class drug (optionally ivermectin), doxycycline or azithromycin and zinc, other combinations and mixtures thereof can be used. For example, add-on medications can also be included to enhance the power of the preventative treatment.

For example, high dose vitamins such as vitamin A, vitamin C, vitamin E, niacin and/or vitamin D or cholecalciferol are included in each dosage or optionally in a weekly, monthly or second weekly treatment. For example, vitamin D or cholecalciferol can be administered at a dosage of between about 500 and 100,000 units, or between about 2,000 and 50,000 units or between about 4,000 units and 25,000 units (or international units, or IU) and/or vitamin C is administered at a dosage of between about 200 to 5000 mg (or international units, or IU).

In alternative embodiments, a dosage formulation as provided herein or administered with a drug combination as provided herein is at least one vitamin or nutritional supplement, wherein optionally the at least one vitamin or nutritional supplement comprises vitamin K, vitamin D or calcifediol (optionally D2 (or ergocalciferol) or Vitamin D3 or cholecalciferol), optionally administered at about 1000 to 4000 ugm/day), vitamin B6 (or pyridoxine), vitamin B12, vitamin E, and/or vitamin C (optionally administered at 500 mg bid); a flavonoid, plant flavonol or quercetin optionally administered at between about 250 to 500 mg bid. In alternative embodiments, the at least one vitamin, and optionally the at least one vitamin comprises: vitamin C optionally formulated or administered at a dosage of between about 500 to 5000 units (U) per dose, and/or Vitamin D (or cholecalciferol) optionally formulated or administered at a dosage of between about 3,000 to 100,000 units per day, or between about 10,000 to 50,000 units a day.

In alternative embodiments, a mucolytic therapy or drug, optionally acetylcysteine, ambroxol, bromhexine (or BISOLVON™), carbocisteine, erdosteine, mecysteine or dornase alfa, or an expectorant, optionally guaifenesin is formulated with a therapeutic combination as provided herein or is administered with (or before or after) a therapeutic combination as provided herein. In alternative embodiments, the mucolytic therapy or drug is used or formulated as a liquid, capsule or tablet or equivalent, and can have a direct anti-viral activity. In alternative embodiments, a mucolytic therapy or drug is added on to any therapeutic combination as provided herein, for example, and exemplary triple therapy, in a similar way as vitamin D and/or vitamin D is used to enhance eradication of the pathogen, for example, a virus such as a coronavirus, or COVID-19.

In alternative embodiments, another drug is or other drugs are formulated with a therapeutic combination as provided herein or administered with (or before or after) a therapeutic combination as provided herein, can comprise:
a thiazolide class drug, optionally nitazoxanide (or ALINIA™, NIZONIDE™) or tizoxanide (or 2-Hydroxy-N-(5-nitro-2-thiazolyl)benzamide);
molnupiravir, optionally co-administered with and/or formulated with an avermectin class drug (optionally ivermectin), an antibiotic (optionally doxycycline or azithromycin) and/or zinc, or co-administered with and/or formulated with ivermectin, hydroxychloroquine, an antibiotic (optionally doxycycline or azithromycin) and/or zinc;
a mucolytic therapy or drug, optionally acetylcysteine, ambroxol, bromhexine (or BISOLVON™), carbocisteine, erdosteine, mecysteine or dornase alfa, or an expectorant, optionally guaifenesin;
an H2 antagonist, or H2RA, or H2 blockers, or a compound, drug or formulation that decreases stomach acid production or decreases stomach pH, wherein optionally the compound, drug or formulation comprises famotidine (or PEPCID™), ranitidine (or ZANTAC™), nizatidine (or AXID™ or TAZAC™), roxatidine acetate, lafutidine, or cimetidine (or TAGAMET™), and optionally the famotidine is administered at a dosage of between about 10 to 60 mg per day, or between about 20 to 40 mg per day;
a dendrimer, optionally astodrimer sodium (Starpharma, Melbourne, Australia);
an antihistamine class drug such as azelastine, or ASTELIN™, OPTIVAR™, ALLERGODIL™, bepotastine (or TALION™, BEPREVE™), brompheniramine, fexofenadine or ALLEGRA™, pheniramine or AVIL™, or chlorpheniramine;
a selective serotonin reuptake inhibitor (SSRI) class drug, optionally fluvoxamine, or LUVOX™, FAVERIN™, FLUVOXIN™;
a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™, or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™ or ronifibrate, or simfibrate or CHOLESOLVIN™, or any combination thereof, clofazimine, or LAMPENE™;
a combination of clofazimine, fluvoxamine, and zinc (optionally a zinc sulphate, acetate, gluconate or picolinate, or zinc oxide nanoparticles, optionally at a dosage of between about 1 mg to 250 mg);
a combination of an avermectin class drug (optionally ivermectin), clofazimine, fluvoxamine and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally further comprising zinc (optionally a zinc sulphate, acetate, gluconate or picolinate, or zinc oxide nanoparticles, optionally at a dosage of between about 1 mg to 250 mg);
hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);
a corticosteroid or glucocorticoid class drug such as ciclesonide (or ALVESCO™ OMNARIS™, OMNIAIR™, ZETONNA™ or ALVESCO™), budesonide (optionally RHINOCORT™ or PULMICORT™), prednisolone (or ORAPRED™), methyl-prednisolone, prednisone (or DELTASONE™ or ORASONE™) or hydrocortisone (or CORTEF™), wherein optionally the corticosteroid or glucocorticoid class drug (optionally ciclesonide) is inhaled;
an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin E; vitamin A; and/or, vitamin C (optionally administered at 500 mg bid);
favipiravir (or T-705, avigan, or favilavir), optionally at 800 mg bid;
zinc (optionally a zinc sulphate, acetate, gluconate or picolinate) or zinc oxide nanoparticles, optionally at a dosage of between about 1 mg to 250 mg;
colchicine, or COLCRYS™, MITIGARE™;
at least one antibiotic or anti-viral (wherein optionally the antibiotic is doxycycline (optionally, DORYX™, DOXYHEXA™, DOXYLIN™) (optionally formulated or administered at a dosage of between about 25 mg to 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc (optionally a zinc sulphate, acetate, gluconate or picolinate, or zinc oxide nanoparticles, optionally at a dosage of between about 1 mg to 250 mg, or about 50 mg per day), and optionally also including colchicine;

a viral, or a coronavirus or a COVID-19, protease inhibitor, wherein optionally the protease inhibitor comprises: ASC09 (CAS registry no. 1000287-05-7) (Janssen Research and Development, LLC), ritonavir (optionally NORVIR™) or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir, cobicistat or darunavir and cobicistat, or PF-07321332 (or nirmatrelvir, or the combination of nirmatrelvir and ritonavir, or PAXLOVID™), PF-07304814 or PF-008335231 (Pfizer), or remdesivir (for example, GS-5734™, Gilead Sciences) or ritonavir (optionally NORVIR™) in combination with PF-07321332 (or nirmatrelvir, or the combination of nirmatrelvir and ritonavir, or PAXLOVID™), PF-07304814 or PF-008335231 (Pfizer) optionally as an oral formulation, for example, as a tablet or a capsule,

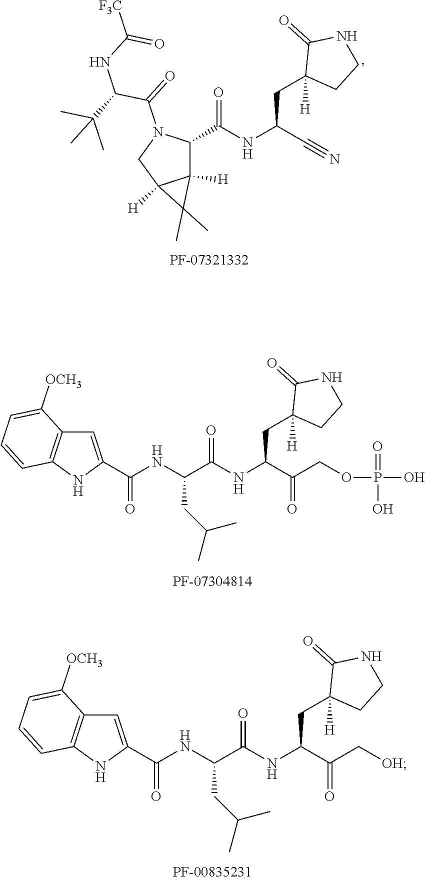 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C ; COc1cccc2[nH]c(C(=O)N[C@@H](CC(C)C)C(=O)N[C@@H](C[C@@H]3CCNC3=O)C(=O)COP(=O)(O)O)cc12 ; COc1cccc2[nH]c(C(=O)N[C@@H](CC(C)C)C(=O)N[C@@H](C[C@@H]3CCNC3=O)C(=O)CO)cc12]

a blood clot inhibiting drug such as aspirin, warfarin (or COUMADIN™) or rivaroxaban (or XARELTO™);
lopinavir, ritonavir (optionally NORVIR™) or the combination lopinavir and ritonavir (optionally NORVIR™, KALETRA™, ALTERA™, ALUVIA™ KALMELTREX, or LOPIMUNE™), opaganib (or YELIVA™), oseltamivir (or TAMIFLU™), and/or zanamivir (or RELENZA™);
an inhibitor or S-phase kinase-associated protein 2 (SKP2), or dioscin, or niclosamide, or NICLOCIDE™, FENASAL™, or PHENASAL™;
a tyrosine kinase inhibitor (TKi), wherein the TKi comprises: masitinib (or MASIVET™, or KINAVET™); or imatinib (or GLEEVEC™, GLIVEC™); or gefitinib (or IRESSA™), or erlotinib (or TARCEVA™), or dasatinib (or SPRYCEL™ DASANIX™);
ribavirin or tribavirin (or COPEGUS™, REBETOL™, or VIRAZOLE™), interferon beta 1b, or a combination of ribavirin and interferon beta, or a combination of lopinavir and ritonavir (optionally NORVIR™) and interferon-beta-1b;
a nucleoside analog reverse-transcriptase inhibitor (NRTI) (optionally abacavir, or ZIAGEN™), acyclovir or aciclovir (optionally ZOVIRAX™), adefovir (optionally HEPSERA™), amantadine (optionally GOCOVRI™ SYMADINE™, SYMMETREL™), rintatolimod (or AMPLIGEN™), amprenavir (optionally, AGENERASE™), aprepitant (or EMEND™), umifenovir (or ARBIDOL™), atazanavir (or REYATAZ™), tenofovir, a combination of efavirenz and emtricitabine and tenofovir (or ATRIPLA™), balavir, baloxavir marboxil (XOFLUZA™), bepotastine (or TALION™, BEPREVE™), bevirimat, bictegravir, biktarvy, brilacidin, cidofovir, caspofungin, lamivudine and zidovudine (optionally, COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir (or VIRACEPT™), nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (optionally, BCX4430, IMMUCILLIN-ATM), remdesivir (optionally, GS-5734™, Gilead Sciences), cytarabine, gemcitabine, emtricitabine, lamivudine, zalcitabine, entecavir, stavudine, telbivudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir (optionally NORVIR™), saquinavir, sofosbuvir, stavudine, telaprevir, tegobuv, tenofovir (optionally tenofovir alafenamide, tenofovir disoproxil or VIREAD™), tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™) or any combination thereof;
fenofibrate, or TRICOR™, FENOBRAT™, FENOGLIDE™, or LIPOFEN™, or a combination of fenofibrate and simvastatin, or CHOLIB™;
an antibody or antibody or vaccine therapy for treating, preventing or ameliorating a microbial or a viral infection (optionally a coronavirus infection, optionally a COVID-19 infection) or a microbial infection (optionally a protozoan, helminthiasis, insect and/or parasitic infection), and optionally the antibody comprises a monoclonal antibody, and optionally the monoclonal antibody comprises sotrovimab (GlaxoSmithKline and Vir Biotechnology), or casirivimab, imdevimab or casirivimab and imdevimab (REGEN-COV™) (Regeneron), or bamlanivimab oretesevimab or bamlanivimab and etesevimab (Junshi Biosciences), or tocilizumab or ACTEMRA™ or ROACTEMRA™ (Hoffmann-La Roche),
and optionally the antibody or vaccine therapy comprises tozinameran or COMIRNATY™ (Pfizer), or elasomeran or SPIKEVAX™ (Moderna), or SPUTNIK V™ or Gam-COVID-Vac (Gamaleya Research Institute), or AZD1222 or COVISHIELD™ or VAXZEVRIA™ (Oxford-AstraZeneca),
and optionally the antibody or antibody therapy comprises or is contained in a convalescent sera or plasma, and/or
any combination thereof.

In alternative embodiments, glucocorticoids or corticoid steroids such as dexamethasone (for example, at between about 1 to 20 mg) or prednisone (for example, at between about 1 to 50 mg), particularly in or for patients who are debilitated, is added to an exemplary formulation or is also administered in the single dose for example once a fortnight or once a month to stimulate adrenal effects; it is known that dexamethasone helps control the body's cytokine storm and so it will be able to suppress the cytokine release in those patients who have a symptomatic carrier status or may be inadvertently infected and already releasing cytokines.

In alternative embodiments, the direct inclusion of hydroxychloroquine permits the avermectin class drug (optionally ivermectin) to be present as a long release agent, and hydroxychloroquine combined in a dose of between about 20 to 1000 mg with a half-life of 40 days gives a double protective method of control or prophylaxis for, for example, front line workers exposed to infected patients.

In alternative embodiments, of methods as provided herein, administered is a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc (optionally a zinc sulphate, acetate, gluconate or picolinate, or zinc oxide nanoparticles, optionally at a dosage of between about 1 mg to 250 mg, or about 50 mg per day), which optionally can be administered:
(a) once a month; or
(b) for the first four, five, six or seven days of treatment an avermectin class drug (optionally ivermectin) is given at about 24 mg per day or between about 20 to 30 mg per day, doxycycline or azithromycin is given at about 100 mg per day or between about 50 and 150 mg per day, clofazimine is given about 100 mg per day or between about 50 and 150 mg per day, and zinc (optionally a zinc sulphate, acetate, gluconate or picolinate, or zinc oxide nanoparticles) is administered at a dosage of between about 25 mg to 100 mg per day, or about 50 mg per day,
and after this initial first four, five, six or seven days of treatment a once a month maintenance regimen of an avermectin class drug (optionally ivermectin) dosaged at between about 60 to 80 mg, or about 60 mg, clofazimine dosaged at about 100 mg or between about 50 to 150 mg, doxycycline or azithromycin dosaged at about 100 mg or between about 50 to 150 mg, and zinc (optionally a zinc sulphate, acetate, gluconate or picolinate, or zinc oxide nanoparticles) is administered at a dosage of between about 25 mg to 100 mg per day, or about 50 mg per day) is given,
and optionally the avermectin class drug (optionally ivermectin), clofazimine, doxycycline or azithromycin and zinc are formulated and administered in separate dosage units (optionally geltabs, tablets, capsules), or the avermectin class drug (optionally ivermectin), clofazimine, doxycycline or azithromycin and zinc are formulated and administered in one unit dosage (optionally all in one a geltab, tablet, capsule).

In an embodiment, there is provided a method of treatment of coronavirus infection, the method comprising administering the to an individual in need thereof a combination comprising nirmatrelvir and ribavirin. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided a method of treatment of coronavirus infection, the method comprising administering the to an individual in need thereof a combination comprising disulfiram, or a combination comprising disulfiram and hydroxychloroquine. The combination may further comprise zinc. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided a method of treatment of coronavirus infection, the method comprising administering the to an individual in need thereof a combination comprising fenofibrate. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided a method of treatment of coronavirus infection, the method comprising administering the to an individual in need thereof a combination comprising amantadine. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided a combination comprising nirmatrelvir and ribavirin for use in treating a coronavirus infection. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided a combination comprising disulfiram, or a combination comprising disulfiram and hydroxychloroquine for use in treating a coronavirus infection. The combination may further comprise zinc. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided a combination comprising fenofibrate for use in treating a coronavirus infection. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided use of a combination comprising amantadine in the manufacture of a medicament for treating a coronavirus infection. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided use of a combination comprising nirmatrelvir and ribavirin in the manufacture of a medicament for treating a coronavirus infection. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided use of a combination comprising disulfiram, or a combination comprising disulfiram and hydroxychloroquine in the manufacture of a medicament for treating a coronavirus infection. The combination may further comprise zinc. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided use of a combination comprising fenofibrate in the manufacture of a medicament for treating a coronavirus infection. The combination may further comprise ivermectin and zinc. The combination may further additionally comprise doxycycline or azithromycin. The combination may further additionally comprise vitamin D. The components of the combination may be provided in dosages as described in any of the above embodiments.

In an embodiment, there is provided use of a combination comprising amantadine in the manufacture of a medicament for for use in treating a coronavirus infection. The combination may further comprise ivermectin and zinc. The combination may further additionally comprise doxycycline or azithromycin. The combination may further additionally comprise vitamin D. The components of the combination may be provided in dosages as described in any of the above embodiments

### Nanoparticles, Nanolipoparticles and Liposomes

Also provided are nanoparticles, nanolipoparticles, vesicles and liposomal membranes comprising compounds or mixtures of compounds as provided herein or used to practice methods as provided herein. For example, in alternative embodiments, an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™) is formulated and/or administered in a liposome or nanoparticle formulation.

In alternative embodiments, provided are multilayered liposomes comprising compounds or mixtures of compounds used to practice methods as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, for example, as described in Park, et al., U.S. Pat. Pub. No. 20070042031, including method of producing a liposome by encapsulating an active agent, the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, for example, for targeting delivery of a compound, as described for example, in U.S. Pat. Pub. No. 20070110798.

The invention also provides nanoparticles comprising compounds used to practice methods as provided herein in the form of active agent-containing nanoparticles (for example, a secondary nanoparticle), as described, for example, in U.S. Pat. Pub. No. 20070077286. In one embodiment, provided are nanoparticles comprising a fat-soluble active agent of this invention or a fat-solubilized water-soluble active agent to act with a bivalent or trivalent metal salt.

In one embodiment, solid lipid suspensions can be used to formulate and to deliver compositions used to practice methods as provided herein to mammalian cells in vivo, in vitro or ex vivo, as described, for example, in U.S. Pat. Pub. No. 20050136121.

### Products of Manufacture and Kits

Provided are compositions, including preparations, formulations and/or kits, comprising combinations of ingredients, for example, therapeutic combinations as described herein. In alternative embodiments, therapeutic combination can be mixed and administered together, or alternatively, they can be an individual member of a packaged combination of ingredients, for example, a liquid component and a solid product component manufactured in a separate compartment, package, kit or container; for example, where all or a subset of the combinations of ingredients are manufactured in a separate compartment, package or container. In alternative aspects, the package, kit or container comprises a blister package, a clamshell, a tray, a shrink wrap and the like.

For example in an exemplary embodiment, the package, kit or container comprises a blister package, a clamshell, a tray, a shrink wrap and the like contains a first delivery packet (or, what is indicated or configured on the package, kit or container comprises a blister package, a clamshell, a tray, a shrink wrap and the like to be the first dosage taken by the individual) comprising a loading dosage of an avermectin class drug (optionally ivermectin) dosaged at about 30 mg/kg (or 30 mg per 2.2 pounds (lb)) or about 1600 mg to 1800 mg in a 60 kg (about 132 lb) person. In alternative embodiments, a loading dosage of the avermectin class drug (optionally ivermectin) is between about 30 to 60 mg/kg or is between about 1800 mg to 3600 mg in a 60 kg (about 132 lb) person.

In alternative embodiments, further delivery packets contain a maintenance dosage of an avermectin class drug (optionally ivermectin), which can be between about between about 20 mcg/kg (μ/kg) to 5000 mcg/kg (μ/kg) or between about 200 to 2000 mcg/kg (μ/kg) dose, where 200 mcg/kg is equivalent to 12 mg in a 60 kg adult, and 2000 mcg/kg is 120 mg which is 7% of the LD₅₀.

In alternative embodiments, in addition to a loading dosage of an avermectin class drug (optionally ivermectin), a first delivery packet, and further delivery packets, also contain doxycycline or azithromycin and zinc.

In alternative embodiments, a first delivery packet, and further delivery packets, also contain an additional drug or drugs, for example as provided herein, for example, a vitamin (such as vitamin A, D, C, E or niacin), hydroxychloroquine, a hydrocortisone or cortisol (optionally CORTEF™, SOLUCORTEF™), optionally hydrocortisone sodium succinate or hydrocortisone acetate or dexamethasome (optionally DEXTENZA™, OZURDEX™ NEOFORDEX™), an H2 antagonist such as famotidine, and the like, which can be configured in the package, kit or container comprises a blister package, a clamshell, a tray, a shrink wrap and the like to be taken sequentially or in an overlapping or staggered dosage regimen.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are for blister packs, clamshells or trays comprising a drug combination or formulation as provided herein, or a drug combination, pharmaceutical preparations or pharmaceutical compositions used to practice methods as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack comprises a molded PVC base, with raised areas (the “blisters”) to contain the tablets, pills, etc. comprising the combinations of drugs drug combination, or formulations, pharmaceutical preparations or pharmaceutical compositions used in methods as provided herein, covered by a foil laminate. Tablets, pills, etc. can be removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided is a method of packaging wherein the compositions comprising combinations of ingredients are contained in-between a card and a clear PVC. The PVC can be transparent so the item (pill, tablet, geltab, etc.) can be seen and examined easily; and in one aspect, can be vacuum-formed around a mold so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, for example, for items such as pills, tablets, geltabs, etc. are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components: a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (for example, using an AERGO 8 DUO™, SCA Consumer Packaging, Inc., DeKalb IL) using regular heat seal tooling. This alternative aspect, using heat seal tooling, can seal common types of thermoformed packaging.

In alternative embodiments, therapeutic combinations and formulations drug combination, or pharmaceutical preparations or pharmaceutical compositions used in methods drug combination, are formulated, for example, as a powder, for example, as lyophilized material, for example, a lyophilized encapsulated product, for example, for practicing methods as provided herein, can be packaged alone or in combinations, for example, as “blister packages” or as a plurality of packettes, including as lidded blister packages, lidded blister or blister card or packets or packettes, or a shrink wrap, or kits, and the like.

In alternative embodiments, laminated aluminum foil blister packs are used, for example, for the preparation of therapeutic combinations or formulations as provided herein, or for pharmaceutical preparations or pharmaceutical compositions used in methods as provided herein. Products or kits comprise an aqueous solution(s) which are dispensed (for example, by measured dose) into containers. Trays can be freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, for example, using hard temper aluminum (for example, alufoil) lidding material. In one aspect, hermetically-sealed high barrier aluminum (for example, alufoil) laminates are used. In one aspect, products of manufacture include kits or blister packs, use foil laminations and strip packs, stick packs, sachets and pouches, peelable and non-peelable laminations combining foil, paper, or film for high barrier packaging.

In alternative embodiments, multi-component products of manufacture, including kits or blister packs as provided herein, include memory aids to help remind patients when and how to take the therapeutic combination. This safeguards the therapeutic combination's efficacy by protecting each tablet, geltab or pill until it's taken; gives the product or kit portability, makes it easy to take a dose anytime or anywhere.

### Dosaging and Packaging for Therapeutic or Prophylactic Purposes

In alternative embodiments, provided are drug combinations and drug delivery devices comprising these combinations for prophylactic (prevention) purposes.

In an exemplary embodiment, a first dosage taken by the individual comprises a loading dosage of an avermectin class drug (optionally ivermectin) dosaged at about 300 μg/kg to 30 mg/kg (or 30 mg per 2.2 pounds (lb)) or about 18 mg to 1800 mg in a 60 kg (about 132 lb) person. In alternative embodiments, a loading dosage of an avermectin class drug (optionally ivermectin) is between about 30 μg/kg to 60 mg/kg or is between about 18 mg to about 1200 mg or 1800 mg in a 60 kg (about 132 lb) person.

In alternative embodiments, further administered dosages comprise a maintenance dosage of an avermectin class drug (optionally ivermectin), which can be between about between about 20 mcg/kg (μ/kg) to 5000 mcg/kg (μ/kg) or between about 200 to 2000 mcg/kg (μ/kg) dose, where 200 mcg/kg is equivalent to 12 mg in a 60 kg adult, and 2000 mcg/kg is 120 mg which is 7% of the LD₅₀.

In alternative embodiments, in addition to a loading dosage of the avermectin class drug (optionally ivermectin), further drugs such as an antibiotic or anti-viral such as for example doxycycline or azithromycin, and/or zinc (for example, zinc picolinate acid, or a zinc sulphate, acetate, gluconate or picolinate salt, or other zinc salts or zinc-comprising compounds) are also administered, and in alternative embodiments additional drugs or vitamins or nutritional supplements such as chloroquine (or ARALEN™), chloroquine phosphate, chloroquine diphosphate and/or hydroxychloroquine (HCQ) (optionally, PLAQUENIL™), and/or a vitamin (such as vitamin C, D, E, A, K or niacin) is/are also administered.

In alternative embodiments, an additional drug or drugs and/or vitamins and/or nutritional supplements (for example, selenium or copper) are administered after the initial loading dose of the avermectin class drug (optionally ivermectin), for example additional drug or drugs as provided herein, for example, a vitamin (such as vitamin C, D, E, A, K or niacin), hydroxychloroquine, an H2 antagonist such as famotidine, chloroquine (or ARALEN™), chloroquine phosphate, chloroquine diphosphate and/or hydroxychloroquine (HCQ) (optionally, PLAQUENIL™), and the like, administered sequentially or in an overlapping or staggered dosage regimen.

For example, in an exemplary embodiment, a loading dosage of the avermectin class drug (optionally ivermectin) is taken with doxycycline or azithromycin and zinc followed by (the next day, or after 2, 3, 4, 5, 6, 7, 8, 9, 10, 11, or 12 or more days) an additional or maintenance dosage of the avermectin class drug (optionally ivermectin) and/or doxycycline or azithromycin and zinc and/or with an additional drug or drugs as provided herein.

In alternative embodiments, a therapeutic or a prophylactic drug or ingredient combination “package”, which can be a kit, a blister pack, a clamshell, a nebulizer, an inhaler, a respirator or a CPAP insert, or the like, is designed such that a particular drug or ingredient combination (for example, a drug or ingredient combination have 2, 3, 4, 5, or 6 ingredients or active agents, wherein one, several or all are separately formulated or formulated into one delivery agent such as a capsule or geltab, or nebulizer, inhaler, respirator or CPAP insert), to be taken by a user every day, every other day, every week, every two weeks or every 4 weeks (i.e., monthly). In alternative embodiments, the therapeutic or a prophylactic drug combination “package” is designed (for example, instructing the user) to take the drug combination as a staggered dosage, for example, one administration of the drug combination for two or three days in a row staggered by a week before the next two or three day administration cycle begins again.

Any of the above aspects and embodiments can be combined with any other aspect or embodiment as disclosed here in the Summary, Figures and/or Detailed Description sections.

### Definitions

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

As used herein, the term “comprising” means “including.” Variations of the word “comprising”, such as “comprise” and “comprises,” have correspondingly varied meanings.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

Embodiments of the invention are set forth in the claims as set forth below.

The invention will be further described with reference to the examples described herein; however, it is to be understood that the invention is not limited to such examples.

### EXAMPLES

### Example 1

A patient presents with:
at least one, or two, or several of these symptoms: fever or chills, cough, shortness of breath or difficulty breathing, fatigue, muscle or body aches, headache, new loss of taste or smell, sore throat, congestion or runny nose, nausea or vomiting, diarrhea; or,
the patient believes there is a high likelihood they have been exposed to COVID-19, or
the patient has come in close contact with an individual with COVID-19, or
the patient has recently tested positive for COVID-19, or any variant thereof, including the delta or omicron COVID variant,
thereupon they are treated with one of the following exemplary treatment regimens:
(1) patient is administered:
(a) proguanil (also called chlorguanide, or PALUDRINE™), or atovaquone (or MEPRON™), or a combination of proguanil and atovaquone (or MALARONE™), starting immediately for 3 to 10 days,
wherein the proguanil, atovaquone or the combination of proguanil and atovaquone are orally administered, optionally as tablets or capsules, and the unit dosage of atovaquone is 250 mg, 300 mg, 350 mg, 400 mg, 500 mg or 1 gram, and the unit dosage of proguanil is 100 mg, 250 mg, 300 mg, 350 mg or 400 mg, and
(b) patient is also administered (with 1(a)):
   ivermectin, optionally dosage at about 300 μg/kg to 30 mg/kg (or 30 mg per 2.2 pounds (lb)) or about 18 mg to 1800 mg in a 60 kg (about 132 lb), or is dosage at 50 μg/kg, 75 μg/kg or 100 μg/kg, or at a loading dose of ivermectin of between about 300 μg/kg to between 30 mg/kg to 60 mg/kg or between about 18 mg to about 1200 mg or 1600 mg to 1800 mg in a 60 kg (about 132 lb) person, or between about 300 μg (mcg) to about 40 to 70 mg/kg, or a dosage of 60 to 120 mg to about 1600 to 1800 mg for an adult; or, (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult,
   hydroxychloroquine (optionally, PLAQUENIL™), optionally formulated or administered at a dosage of between about 10 mg to 2000 mg per day,
   doxycycline (optionally, DORYX™, DOXYHEXA™, DOXYLIN™) (optionally formulated or administered at a dosage of between about 25 mg to about 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg),
   vitamin D (optionally vitamin D2, or ergocalciferol, or Vitamin D3 or cholecalciferol, optionally administered at about 1000 to 4000 ugm/day) and/or vitamin C, and/or
   a zinc, a zinc chelate or a zinc salt, or a zinc sulphate, zinc acetate, zinc gluconate or zinc picolinate, optionally formulated or administered at a dosage of between about 1 mg to 250 mg,
and in one exemplary treatment regimen the patient is administered a therapeutic combination comprising: ivermectin 12 mg, doxycycline 100 mg and zinc chelate 25 mg or optionally administered at about 1000 to 4000 ugm/day,
and in one exemplary treatment regimen the patient is administered a therapeutic combination comprising: proguanil and atovaquone (or MALARONE™), hydroxychloroquine, vitamin D (optionally vitamin D2, or ergocalciferol, optionally administered at about 1000 to 4000 ugm/day), and zinc, a zinc chelate or a zinc salt at a unit dosage of 25 mg or optionally administered at about 1000 to 4000 ugm/day,
and in one exemplary treatment regimen the patient is administered a therapeutic combination comprising: proguanil and atovaquone (or MALARONE™), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine, ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine, ivermectin, vitamin D (optionally vitamin D2, or ergocalciferol, optionally administered at about 1000 to 4000 ugm/day), vitamin C, and zinc, a zinc chelate or a zinc salt at a unit dosage of 25 mg or optionally administered at about 1000 to 4000 ugm/day; or
(2) patient is administered:
(a) a nicotinic antagonist, a dopamine agonist or a noncompetitive N-Methyl-D-aspartate (NMDA) antagonist, optionally amantadine, or GOCOVRI™, or SYIVIADINE™, or SYMMETREL™, optionally dosaged at between about 100 to 200 mg per dose, optionally formulated as tablets or capsules, and
(b) patient is also administered (with 2(a)):
   ivermectin, optionally dosage at about 300 μg/kg to 30 mg/kg (or 30 mg per 2.2 pounds (lb)) or about 18 mg to 1800 mg in a 60 kg (about 132 lb), or is dosage at 50 pg/kg, 75 pg/kg or 100 μg/kg, or at a loading dose of ivermectin of between about 300 μg/kg to between 30 mg/kg to 60 mg/kg or between about 18 mg to about 1200 mg or 1600 mg to 1800 mg in a 60 kg (about 132 lb) person, or between about 300 μg (mcg) to about 40 to 70 mg/kg, or a dosage of 60 to 120 mg to about 1600 to 1800 mg for an adult; or, (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult,
   hydroxychloroquine (optionally, PLAQUENIL™), optionally formulated or administered at a dosage of between about 10 mg to 2000 mg per day,
   doxycycline (optionally, DORYX™, DOXYHEXA™, DOXYLIN™) (optionally formulated or administered at a dosage of between about 25 mg to about 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg),
   vitamin D (optionally vitamin D2, or ergocalciferol, or Vitamin D3 or cholecalciferol, optionally administered at about 1000 to 4000 ugm/day) and/or vitamin C, and/or
   a zinc, a zinc chelate or a zinc salt, or a zinc sulphate, zinc acetate, zinc gluconate or zinc picolinate, optionally formulated or administered at a dosage of between about 1 mg to 250 mg,
and in one exemplary treatment regimen the patient is administered a therapeutic combination comprising: ivermectin 12 mg, doxycycline 100 mg and zinc chelate 25 mg,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day,), vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), vitamin C, and zinc, a zinc chelate or a zinc salt; or
(3) patient is administered:
(a) an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
and optionally the DDS system comprises: a polyether ester urethane comprising 65% D, L-lactide, 19% polyethylene glycol, and 16% glycolide interlinked with an aliphatic di-isocyanate, or comprises VISCOPRENE™,
and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously, and
(b) patient is also administered (with 3(a)):
   ivermectin, optionally dosage at about 300 μg/kg to 30 mg/kg (or 30 mg per 2.2 pounds (lb)) or about 18 mg to 1800 mg in a 60 kg (about 132 lb), or is dosage at 50 pg/kg, 75 pg/kg or 100 μg/kg, or at a loading dose of ivermectin of between about 300 μg/kg to between 30 mg/kg to 60 mg/kg or between about 18 mg to about 1200 mg or 1600 mg to 1800 mg in a 60 kg (about 132 lb) person, or between about 300 μg (mcg) to about 40 to 70 mg/kg, or a dosage of 60 to 120 mg to about 1600 to 1800 mg for an adult; or, (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; or
   hydroxychloroquine (optionally, PLAQUENIL™), optionally formulated or administered at a dosage of between about 10 mg to 2000 mg per day,
   doxycycline (optionally, DORYX™, DOXYHEXA™, DOXYLIN™) (optionally formulated or administered at a dosage of between about 25 mg to about 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg),
   vitamin D (optionally vitamin D2, or ergocalciferol, or Vitamin D3 or cholecalciferol, optionally administered at about 1000 to 4000 ugm/day) and/or vitamin C, and/or
   a zinc, a zinc chelate or a zinc salt, or a zinc sulphate, zinc acetate, zinc gluconate or zinc picolinate, optionally formulated or administered at a dosage of between about 1 mg to 250 mg,
and in one exemplary treatment regimen the patient is administered a therapeutic combination comprising: ivermectin 12 mg, doxycycline 100 mg and zinc chelate 25 mg,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day,), vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), vitamin C, and zinc, a zinc chelate or a zinc salt; or
(4) patient is administered:
(a) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™ or combination of bezafibrate and chenodeoxycholic acid, or HEPACONDA™, or aluminium clofibrate, or alfibrate, or ciprofibrate, or clinofibrate or LIPOCLIN™, or clofibrate or ATROMID-S™, or clofibride, or gemfibrozil or LOPID™, or ronifibrate, or simfibrate or CHOLESOLVIN™, or any combination thereof, and
(b) patient is also administered (with 4(a)):
   ivermectin, optionally dosage at about 300 μg/kg to 30 mg/kg (or 30 mg per 2.2 pounds (lb)) or about 18 mg to 1800 mg in a 60 kg (about 132 lb), or is dosage at 50 pg/kg, 75 pg/kg or 100 μg/kg, or at a loading dose of ivermectin of between about 300 μg/kg to between 30 mg/kg to 60 mg/kg or between about 18 mg to about 1200 mg or 1600 mg to 1800 mg in a 60 kg (about 132 lb) person, or between about 300 μg (mcg) to about 40 to 70 mg/kg, or a dosage of 60 to 120 mg to about 1600 to 1800 mg for an adult; or, (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; or
   hydroxychloroquine (optionally, PLAQUENIL™), optionally formulated or administered at a dosage of between about 10 mg to 2000 mg per day,
   doxycycline (optionally, DORYX™, DOXYHEXA™, DOXYLIN™) (optionally formulated or administered at a dosage of between about 25 mg to about 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg),
   vitamin D (optionally vitamin D2, or ergocalciferol, or Vitamin D3 or cholecalciferol, optionally administered at about 1000 to 4000 ugm/day) and/or vitamin C, and/or
   a zinc, a zinc chelate or a zinc salt, or a zinc sulphate, zinc acetate, zinc gluconate or zinc picolinate, optionally formulated or administered at a dosage of between about 1 mg to 250 mg,
and in one exemplary treatment regimen the patient is administered a therapeutic combination comprising: ivermectin 12 mg, doxycycline 100 mg and zinc chelate 25 mg,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day,), vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), vitamin C, and zinc, a zinc chelate or a zinc salt; or
(5) patient is administered:
(a) an anti-malarial drug, wherein optionally the anti-malarial drug comprises mefloquine (or LARIAM™, MEPHAQUIN™, or MEFLIAM™), wherein optionally the mefloquine is formulated for oral administration, optionally in tablet or capsule form, optionally as 200 mg, 250 mg or 300 mg tablets, and
(b) patient is also administered (with 5(a)):
   ivermectin, optionally dosage at about 300 μg/kg to 30 mg/kg (or 30 mg per 2.2 pounds (lb)) or about 18 mg to 1800 mg in a 60 kg (about 132 lb), or is dosage at 50 pg/kg, 75 pg/kg or 100 μg/kg, or at a loading dose of ivermectin of between about 300 μg/kg to between 30 mg/kg to 60 mg/kg or between about 18 mg to about 1200 mg or 1600 mg to 1800 mg in a 60 kg (about 132 lb) person, or between about 300 pg (mcg) to about 40 to 70 mg/kg, or a dosage of 60 to 120 mg to about 1600 to 1800 mg for an adult; or, (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; or
   hydroxychloroquine (optionally, PLAQUENIL™), optionally formulated or administered at a dosage of between about 10 mg to 2000 mg per day,
   doxycycline (optionally, DORYX™, DOXYHEXA™, DOXYLIN™) (optionally formulated or administered at a dosage of between about 25 mg to about 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg),
   vitamin D (optionally vitamin D2, or ergocalciferol, or Vitamin D3 or cholecalciferol, optionally administered at about 1000 to 4000 ugm/day) and/or vitamin C, and/or
   a zinc, a zinc chelate or a zinc salt, or a zinc sulphate, zinc acetate, zinc gluconate or zinc picolinate, optionally formulated or administered at a dosage of between about 1 mg to 250 mg,
and in one exemplary treatment regimen the patient is administered a therapeutic combination comprising: ivermectin 12 mg, doxycycline 100 mg and zinc chelate 25 mg,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day,), vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), vitamin C, and zinc, a zinc chelate or a zinc salt; or
(6) patient is administered:
(a) PF-07321332, or nirmatrelvir, or the combination of nirmatrelvir and ritonavir, or PAXLOVID™, PF-07304814 or PF-008335231 (Pfizer); or remdesivir (or GS-5734™, Gilead Sciences) or ritonavir (optionally NORVIR™) in combination with PF-07321332, PF-07304814 or PF-008335231 (Pfizer), optionally as an oral formulation, optionally as a table or a capsule,
and optionally the PF-07321332, or nirmatrelvir, or the combination of nirmatrelvir and ritonavir, or PAXLOVID™, are administered on a twice daily regimen, optionally for five to ten days, optionally unit doses of the PF-07321332, or nirmatrelvir, or the combination of nirmatrelvir and ritonavir, or PAXLOVID™, is 300 mg, or two 150 mg tablets of the PF-07321332, or nirmatrelvir, or the combination of nirmatrelvir and ritonavir, or PAXLOVID™, with one 100 mg tablet of ritonavir, optionally given twice-daily for five days, or between about 5 to 21 days, and
(b) patient is also administered (with 6(a)):
   ivermectin, optionally dosage at about 300 μg/kg to 30 mg/kg (or 30 mg per 2.2 pounds (lb)) or about 18 mg to 1800 mg in a 60 kg (about 132 lb), or is dosage at 50 pg/kg, 75 pg/kg or 100 μg/kg, or at a loading dose of ivermectin of between about 300 μg/kg to between 30 mg/kg to 60 mg/kg or between about 18 mg to about 1200 mg or 1600 mg to 1800 mg in a 60 kg (about 132 lb) person, or between about 300 μg (mcg) to about 40 to 70 mg/kg, or a dosage of 60 to 120 mg to about 1600 to 1800 mg for an adult; or, (2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; or
   hydroxychloroquine (optionally, PLAQUENIL™), optionally formulated or administered at a dosage of between about 10 mg to 2000 mg per day,
   doxycycline (optionally, DORYX™, DOXYHEXA™, DOXYLIN™) (optionally formulated or administered at a dosage of between about 25 mg to about 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg),
   vitamin D (optionally vitamin D2, or ergocalciferol, or Vitamin D3 or cholecalciferol, optionally administered at about 1000 to 4000 ugm/day) and/or vitamin C, and/or
   a zinc, a zinc chelate or a zinc salt, or a zinc sulphate, zinc acetate, zinc gluconate or zinc picolinate, optionally formulated or administered at a dosage of between about 1 mg to 250 mg,
and in one exemplary treatment regimen the patient is administered a therapeutic combination comprising: ivermectin 12 mg, doxycycline 100 mg and zinc chelate 25 mg,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day,), vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), and zinc, a zinc chelate or a zinc salt,
and in one exemplary treatment regimen the patient is administered a therapeutic combination comprising: proguanil and atovaquone (or MALARONE™), hydroxychloroquine (optionally formulated or administered at a dosage of between about 10 mg to 2000 mg per day), ivermectin, vitamin D (optionally vitamin D2, or ergocalciferol, optionally administered at about 1000 to 4000 ugm/day), vitamin C, and zinc, a zinc chelate or a zinc salt.

### Example 2

A 42-year-old patient with a positive PCR for coronavirus presented with sweating, loss of taste and loss of smell and he estimated symptoms that have been there for 3 days. After obtaining total history, and explaining the potential side effects, he was commenced on nirmatrelvir and ribavirin at a dose of 200 mg twice daily to permit the nirmatrelvir to work better.

It is expected that he will improve and indeed, it is expected that he will improve by day 5-7 and then his swab should be negative by day 14.

### Example 3

A 67-year-old male is infected and is to take medication orally prior to becoming ill enough to go the hospital. He was started on nirmatrelvir but combined with Ribavirin 400 mg twice daily to allow better absorption. The patient is taking these on a daily basis for 5 days before the first swab and the treatment is 10 days to ensure close to 100% cure rate. The patient is cured of the condition.

### Example 4

A 62-year-old male presented with PCR positive for COVID 19. He was quite symptomatic and had fevers and rigors with oximetry reading of 93. He is placed on disulfiram 250 mg twice daily combined with hydroxychloroquine 200 mg twice daily, zinc 25 mg twice daily, and azithromycin 250 twice daily. He had a background dosing of vitamin D 5000 U/day total treatment was for 10 days. He is expected to do very well given the combination therapy with disulfiram.

### Example 5

A 47-year-old male, once vaccinated 6 weeks ago with the Pfizer mRNA vaccine, presented with cough, fever, loss of taste and smell, diarrhoea, aches and pains, and oximetry level of 96%. He was PCR swabbed positive. He only had symptoms for three days, so he was commenced on the combination of nirmatrelvir 150 mg twice daily together with ritonavir 100 mg. The patient's symptoms improved progressively over 6 days with oximetry reading elevated and symptoms slowly diminishing. However, he was not completely improving by day 5 and the therapy continued for yet another three days by which stage his oximetry was up to 97%.

### Example 6

A 72-year old patient, never vaccinated, presented with a two-day history of fevers of 41 degrees, rigors, shakes, sweating, sore throat, aches and pains, and loss of taste and smell. He was PCR swabbed positive. He had a short bout of diarrhoea beforehand. He was commenced on a combination of nirmatrelvir 150 mg twice daily for ten days combined with ritonavir 100 mg twice daily, together with a choice of ivermectin 12 mg twice daily. He was also given vitamin D 10 000 units daily and zinc picolinic acid 50 mg. The patient quickly deteriorated but kept on improving after day 4 and by day 8, his oxygen tensions rose from 94 to 97 and he felt markedly better, although still fatigued. He recovered completely.

### Example 7

A 64-year-old female, two vaccinations three months before, presented with fever, aches and pain, profound tiredness and some loss of taste and smell. She was PCR swabbed positive. She was diagnosed having coronavirus infection. She was commenced on nirmatrelvir 150 mg twice daily for ten days combined with ritonavir 100 mg twice daily to maintain blood levels and was given ivermectin 12 mg bid, zinc 50 mg and doxycycline 100 mg twice daily. This constitutes the drug combination. Within 24 hours, the patient's oxygenation improved dramatically from 92 to 97 and she felt markedly better within three days. The patient wants to stop all medications, but this was prevented, and she continued to day 10. By day 20, her PCR nasopharyngeal swab was negative, and she was cured.

### Example 8

A 34-year-old female front-line worker and was diagnosed with coronavirus in association with fevers, aches and fatigue. She is commenced on amantadine orally using the standard capsule of 137 mg once daily for ten days. This was in combination with Ivermectin 12 mg bd, zinc picolinic acid 50 mg mane, and doxycycline 100 mg bd. She developed a rash to the doxycycline when added and this was changed to azithromycin 250 mg bd for the duration of the ten-day course.

### Example 9

A 71-year-old male presents with symptoms of coronavirus infection with tiredness, rigors, fever, and loss of taste and smell with positive PCR. He was treated with a combination of ivermectin 12 mg 2 in the morning and 2 at night on day one, and 12 mg daily for the rest of the 10 days. Zinc picolinic acid 50 mg mane and doxycycline 100 mg bd together with disulfiram 250 mg twice daily and strictly told to keep away from any alcohol or otherwise he may have nausea and vomiting. His symptoms improved slowly. By day 4 his oxygen tension rose from 93 to 97 but his symptoms kept on improving with about 90% of his symptoms completely improved by day 8. At 20 days, his PCR swab was negative for coronavirus.

### Example 10

A 61-year-old female is infected with swab positive coronavirus. She had previously had vaccination but nevertheless developed infection. Her symptoms were classic with sore throat, coughing, shortness of breath, oxygen tension of 92% and some diarrhoea but no loss of smell or taste. She was commenced on fenofibrate 150 mg twice daily to supplement the ivermectin 12 mg bd, zinc picolinic acid 50 mg mane, doxycycline 150 mg bd. Her partner was also found to be positive and was allergic to doxycycline so this partner was also quite symptomatic with oxygen tension of 93%. The partner was commenced on fenofibrate 150 mg twice daily with ivermectin 12 mg bd, zinc picolinic acid 50 mg mane and azithromycin 250 mg bd. Both were treated for 10 days and both improved quite rapidly with negative PCR at day 18.

A number of embodiments of the invention have been described. Nevertheless, it can be understood that various modifications may be made without departing from the spirit and scope of the invention. Accordingly, other embodiments are within the scope of the following claims.

1. A method for preventing, or substantially preventing, decreasing the chances of having any adverse effects from, decreasing the severity of adverse effects from, or treating or ameliorating a viral infection or a microbial infection, or a protozoan, helminthiasis, insect and/or parasitic infection, in an individual in need thereof, comprising administering to an individual in need thereof a drug or or therapeutic combination or composition comprising:
(a)
(i) a loading dosage comprising an avermectin class drug (optionally ivermectin) in a dosage of:
(1) about 300 μg/kg to 30 mg/kg (or 30 mg per 2.2 pounds (lb)) or about 18 mg to 1800 mg in a 60 kg (about 132 lb), or a loading dose of the avermectin class drug (optionally ivermectin) of between about 300 pg/kg to 30 to 60 mg/kg or between about 18 mg to about 1200 mg or 1600 mg to 1800 mg in a 60 kg (about 132 lb) person, or between about 300 pgm (mcg) to 40 mg/kg or 70 mg/kg, or a dosage of between about 120 mg to 280 mg to about 1600 to 1800 mg for an adult; or
(2) between about 18 to 50 mg, or about 18 mg, 24 mg, 30 mg, 36 mg or 40 mg, or between about 50 mg to 100 mg, or 60 to 120 mg up to about 1600 to 1800 mg for an adult; and
(ii) after administration of the loading dosage of (i), administering a maintenance dosage of ivermectin of between about 20 mcg/kg (μ/kg) to 5000 mcg/kg (μ/kg) or between about 200 to 2000 mcg/kg (μ/kg) per dose, where 200 mcg/kg is equivalent to a 12 mg dosage in a 60 kg adult, and 2000 mcg/kg is equivalent to 120 mg per dose;
(b) a drug, a formulation or a therapeutic combination of drugs comprising an avermectin class drug (optionally ivermectin) at a dosage of:
(1) about 300 μg/kg to 30 mg/kg (or 30 mg per 2.2 pounds (lb)) or about 18 mg to 1800 mg in a 60 kg (about 132 lb), or at a loading dose of the avermectin class drug (optionally ivermectin) of between about 300 pg/kg to 30 to 60 mg/kg or between about 18 mg to about 1200 mg or 1600 mg to 1800 mg in a 60 kg (about 132 lb) person, or between about 300 pgm (mcg) to 40 mg/kg or 70 mg/kg, or a dosage of between about 120 mg to 280 mg to about 1600 to 1800 mg for an adult, or
(2) between about 18 to 50 mg, or about 18 mg, 24 mg, 30 mg, 36 mg or 40 mg, or between about 50 mg to 100 mg, or 60 to 120 mg up to about 1600 to 1800 mg for an adult;
(c) a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan,
wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days;
(d) an anti-androgen drug, and optionally the anti-androgen drug is bicalutamide, optionally CASODEX™, or dutasteride,
and optionally the anti-androgen drug is a nonsteroidal anti-androgen (NSAA) or an androgen receptor (AR) antagonist, and optionally the NSAA or AR antagonist comprises proxalutamide (Suzhou Kintor Pharmaceuticals, Inc., a subsidiary of Kintor Pharmaceutical Limited), or flutamide (or niftolide), or bicalutamide or enzalutamide,
and optionally the anti-androgen drug comprises a 5α-reductase inhibitor, and optionally the 5α-reductase inhibitor comprises finasteride,
and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
and optionally the anti-androgen drug, or NSAA, or bicalutamide, proxalutamide, flutamide or niftolide, bicalutamide, enzalutamide or dutasteride, is administered with an avermectin class drug, or ivermectin, optionally also administered with hydroxychloroquine, zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A),
and optionally the anti-androgen drug, or NSAA, or bicalutamide, proxalutamide, flutamide or niftolide, bicalutamide, enzalutamide or dutasteride, is administered with colchicine (or COLCRYS™, MITIGARE™), and optionally also zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A),
and optionally the anti-androgen drug, or NSAA, or bicalutamide, proxalutamide, flutamide or niftolide, bicalutamide, enzalutamide or dutasteride, is administered with an antibiotic (optionally azithromycin or doxycycline), and optionally also zinc and/or a vitamin (optionally vitamin D (optionally vitamin D2, or ergocalciferol, or Vitamin D3 or cholecalciferol, optionally administered at about 1000 to 4000 ugm/day), or vitamin C, B or A), and optionally also with hydroxychloroquine;
(e) an anti-malarial drug, wherein optionally the anti-malarial drug comprises mefloquine (or LARIAM™, MEPHAQUIN™ or MEFLIAM™), wherein optionally the mefloquine is formulated for oral administration, optionally in tablet or capsule form, optionally as 200 mg, 250 mg or 300 mg tablets;
(f) a peroxisome proliferator-activated receptor (PPAR) agonist, wherein optionally the PPAR agonist comprises fenofibrate, or TRICOR™ FENOBRAT™, FENOGLIDE™ or LIPOFEN™ optionally the PPAR agonist comprises a combination of fenofibrate and pravastatin, or PRAVAFENIX™, or the PPAR agonist comprises bezafibrate, or BEZALIP™ or combination of bezafibrate and chenodeoxycholic acid, or HEPACONDA™, or aluminium clofibrate, or alfibrate, or ciprofibrate, or clinofibrate or LIPOCLIN™ or clofibrate or ATROMID-S™ or clofibride, or gemfibrozil or LOP ID™ or ronifibrate, or simfibrate or CHOLESOLVIN™ or any combination thereof,
(g) an acetaldehyde dehydrogenase inhibitor, optionally disulfiram, or ANTABUS™ or ANTABUSE™ optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
and optionally the DDS system comprises: a polyether ester urethane comprising 65% D, L-lactide, 19% polyethylene glycol, and 16% glycolide interlinked with an aliphatic di-isocyanate, or comprises VISCOPRENE™,
and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously,
(h) a nicotinic antagonist, a dopamine agonist or a noncompetitive N-Methyl-D-aspartate (NMDA) antagonist, optionally amantadine, or GOCOVRI™, or SYMADINE™, or SYMMETREL™, optionally dosaged at between about 100 to 200 mg per dose, optionally formulated as tablets or capsules, or
(i) a mitochondrial sensitizer, optionally proguanil or chlorguanide (or PALUDRINE™), or a malarial cytochrome bc1 complex inhibitor, optionally atovaquone (or MEPRON™), or a combination of proguanil and atovaquone (or MALARONE™), and/or
(j) a drug combination or therapeutic regimen comprising any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i).

2. The method of claim 1, wherein the avermectin class drug comprises: ivermectin, moxidectin, selamectin, a milbemycin (optionally milbemectin, milbemycin oxime, moxidectin or nemadectin), doramectin, eprinomectin or abamectin.

3. The method of claim 1, wherein the drug or drug combination is administered to prevent or substantially prevent, or to treat or ameliorate, or decrease the severity of symptoms or pathology of:
a viral infection, optionally a coronavirus, an influenza virus (optionally an influenza A, B or C), a hepatitis virus, a rous sarcoma virus (RSV), a Paramyxoviridae or measles virus, a Paramyxovirus or mumps virus, a Herpes simplex virus (HSV), a Cytomegalovirus (CMV), a Rubivirus or rubella virus, an Enterovirus, a viral meningitis, a rhinovirus, a human immunodeficiency virus (HIV), a varicella-zoster or chickenpox virus, an Orthopoxvirus or variola or smallpox virus, an Epstein-Barr virus (EBV), an Adenovirus, a Hantavirus, a Flaviviridae or Dengue virus, a Zika virus, or a chikungunya virus infection,
a coronavirus infection, optionally a COVID-19 infection, optionally a COVID-19 variant infection, wherein optionally the COVID-19 variant is a delta or an omicron variant, or the coronavirus infection comprises a Middle East respiratory syndrome virus (MERS-CoV) infection;
malaria that can be caused by a parasite of the genus Plasmodium (optionally P. vivax, P. falciparum, P. malariae, P. ovale, or P. knowlesi);
dengue fever or dengue shock syndrome that can be caused by a virus of the Flaviviridae family or a dengue virus;
hepatitis or hepatocellular carcinoma associated with viral hepatitis that can be caused by a virus of the Flaviviridae family or a virus of the genus Hepacivirus or Hepacivirus C virus or hepatitis C;
filariasis, leprosy or streptocerciasis that can be caused by a parasite of the superfamily Filarioidea (optionally Brugia malayi, Brugia timori, Wuchereria bancrofti, Loa loa, Mansonella streptocerca, Mansonella ozzardi, or Mansonella perstans);
leprosy that can be caused by a parasite of the genus Mycobacterium (optionally M. leprae or M. lepromatosis);
river blindness or onchocerciasis that can be caused by parasitic worms such as parasites of the genus Onchocerca (optionally O. volvulus);
hookworm or roundworm infections that can be caused by parasites of the genus Ancylostoma (optionally A. duodenale or A. ceylanicum) or Necator (optionally N. americanus);
trichuriasis or whipworm infection that can be caused by a parasite of the genus Trichuris (optionally T. trichiura); roundworm or an Ascaris infection that can be caused by Ascaris lumbricoides;
mite-carried infections such as scabies that can be caused by the parasite of the genus Sarcoptes (optionally S. scabiei);
infections such as typhus caused by lice or parasites of the order Phthiraptera (optionally Pediculus humanus capitis);
enterobiasis that can be caused by pinworm or parasites of the genus Enterobius (optionally E. vermicularis); and/or
pulicosis or infections cause by fleas or insects of the order Siphonaptera or of the genus Pulex (optionally P. irritans).

4. The method of claim 1, wherein the loading dose of the avermectin class drug (optionally ivermectin) of between is about 15 to 150 mg/kg, or is about 18, 24, 30, 35, 40, 35, 50, 55, 60, 65, 70, 75, 80, 85, 90, 95, 100, 110 or 120 or more mg/kg.

5. The method of claim 1, wherein the loading dosage is given once, or periodically, optionally every 2, 3, 4, 5, 6, 7, 8, 9, 10, 11, or 12 or more days.

6. The method of claim 1, wherein the maintenance dosage of (a)(ii) is administered 1, 2, 3, 4, 5, 6, 7, 8, 9, 10, 11, 12, 13 or 14 days, or every 3 weeks or every month or every two months or longer after the first loading dosage.

7. The method of claim 1, wherein the maintenance dosage of (a)(ii) is administered every 1, 2, 3, 4, 5, 6, 7, 8, 9, 10, 11, 12, 13 or 14 days, every 3 weeks, or monthly, over the 4 to 8 weeks, 6 to 10 weeks, 8 to 12 weeks, 10 to 20 weeks, 15 to 30 weeks or 20 to 52 weeks, or more, after the initial or loading dose is given.

8. The method of claim 1, wherein: an antibiotic or anti-viral is administered with the loading dosage of the avermectin class drug (optionally ivermectin); zinc or a zinc salt or zinc chelate is administered with the loading dosage of the avermectin class drug (optionally ivermectin); or zinc or zinc chelate or a zinc salt and an antibiotic is administered with the loading dosage of the avermectin class drug (optionally ivermectin),
and optionally a drug combination, optionally formulated as one formulation (for example, as a tablet capsule) comprises: ivermectin, doxycycline and zinc chelate, or comprises: ivermectin 12 mg, doxycycline 100 mg and zinc chelate 25 mg.

9. The method of claim 8, wherein the antibiotic comprises doxycycline, azithromycin or hydroxychloroquine (HCQ).\\

10. The method of claim 1, wherein: an antibiotic or anti-viral is administered with the maintenance dose of the avermectin class drug (optionally ivermectin); zinc or a zinc salt or zinc chelate is administered with the maintenance dosage of the avermectin class drug (optionally ivermectin); or zinc or a zinc salt or zinc chelate and an antibiotic or anti-viral is administered with the maintenance dosage of the avermectin class drug (optionally ivermectin).

11. The method of claim 10, wherein the antibiotic comprises doxycycline, azithromycin or hydroxychloroquine (HCQ).

12. The method of claim 1, wherein an additional drug is or drugs, or therapy, is or are administered with the loading dose and/or the maintenance dose of the avermectin class drug (optionally ivermectin), or before the loading dose and/or the maintenance dose, or any time between administration of the loading dose and the maintenance dose.

13. The method of claim 1, wherein an additional drug or therapy, is or are administered with the drug or drug combination of (a), (b), (c), (d), (e), (f), (g), (h) or (i), or any one or more of the following is administered with the drug combination or therapeutic regimen of any combination of (a) to (i), or a combination of (a) and (b), (a) and (c), (a) and (d), (a) and (e), (a) and (f), (a) and (g), (a) and (h), (a) and (i), (b) and (c), (b) and (d), (b) and (e), (b) and (f), (b) and (g), (b) and (h), (b) and (i), (c) and (d), (c) and (e), (c) and (f), (c) and (g), (c) and (h), (c) and (i), (d) and (e), (d) and (f), (d) and (g), (d) and (h), (d) and (i), (e) and (f), (e) and (g), (e) and (h), (e) and (i), (f) and (g), (f) and (h), (f) and (i), (g) and (h), (g) and (i) and/or (h) and (i) of claim 1:
a thiazolide class drug, optionally nitazoxanide (or Alinia™, Nizonide™) or tizoxanide (or 2-Hydroxy-N-(5-nitro-2-thiazolyl)benzamide);
molnupiravir, optionally co-administered with and/or formulated with an avermectin class drug (optionally ivermectin), an antibiotic (optionally doxycycline or azithromycin) and/or zinc, zinc salt or zinc chelate, or co-administered with and/or formulated with ivermectin, hydroxychloroquine, an antibiotic (optionally doxycycline or azithromycin) and/or zinc, zinc salt or zinc chelate;
a mucolytic therapy or drug, optionally acetylcysteine, ambroxol, bromhexine (or BISOLVON™), carbocisteine, erdosteine, mecysteine or dornase alfa, or an expectorant, optionally guaifenesin;
an H2 antagonist, or H2RA, or H2 blockers, or a compound, drug or formulation that decreases stomach acid production or decreases stomach pH, wherein optionally the compound, drug or formulation comprises famotidine (or PEPCID™), ranitidine (or ZANTAC™), nizatidine (or AXID™ or TAZAC™), roxatidine acetate, lafutidine, or cimetidine (or TAGAMET™), and optionally the famotidine is administered at a dosage of between about 10 to 60 mg per day, or between about 20 to 40 mg per day;
a dendrimer, optionally astodrimer sodium (Starpharma, Melbourne, Australia);
an antihistamine class drug such as azelastine, or ASTELIN™, OPTIVAR™, ALLERGODIL™, bepotastine (or TALION™, BEPREVE™) brompheniramine, fexofenadine or ALLEGRA™, pheniramine or AVIL™, or chlorpheniramine;
a selective serotonin reuptake inhibitor (SSRI) class drug, optionally fluvoxamine, or LUVOX™ FAVERIN™ FLUVOXIN™;
a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™, or the PPAR agonist comprises bezafibrate, or BEZALIP™ or combination of bezafibrate and chenodeoxycholic acid, or HEPACONDA™, or aluminium clofibrate, or alfibrate, or ciprofibrate, or clinofibrate or LIPOCLIN™ or clofibrate or ATROMID-S™ or clofibride, or gemfibrozil or LOP ID™ or ronifibrate, or simfibrate or CHOLESOLVIN™ or any combination thereof, clofazimine, or LAMPENE™ optionally dosaged at about 100 mg per day, or between about 50 mg and 150 mg per day, and optionally also including colchicine;
clofazimine, or LAMPENE™ optionally dosaged at about 100 mg per day, or between about 50 mg and 150 mg per day, and chloroquine (or ARALEN™), chloroquine phosphate, chloroquine diphosphate and/or hydroxychloroquine (HCQ) (optionally, PLAQUENIL™), optionally also comprising zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day) and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid), and optionally also including administration of zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine,
a combination of clofazimine (optionally dosaged at about 100 mg or 150 mg per day, or between about 50 mg and 200 mg per day), fluvoxamine, and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), fluvoxamine and at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K;
vitamin A; vitamin E; and/or, vitamin C (optionally administered at 500 mg bid), and optionally further comprising zinc, zinc salt or zinc chelate (optionally a zinc sulphate, acetate, gluconate or picolinate, or zinc oxide nanoparticles, optionally at a dosage of between about 1 mg to 250 mg), and optionally also including colchicine;
hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);
chloroquine (or ARALEN™), chloroquine phosphate, chloroquine diphosphate and/or hydroxychloroquine (HCQ) (optionally, PLAQUENIL™);
a corticosteroid or glucocorticoid class drug such as such as ciclesonide (or Alvesco™, Omnaris™ Omniair™, Zetonna™ or Alvesco™), budesonide (optionally RHINOCORT™ or PULMICORT™), prednisolone (or ORAPRED™), methyl-prednisolone, prednisone (or DELTASONE™ or ORASONE™) or hydrocortisone (or CORTEF™), or a selective estrogen receptor modulator (SERM), or toremifene (or Fareston™), or clomifene or clomiphene (or CLOMID™, SEROPHENE™), wherein optionally the mode of administration for the corticosteroid or glucocorticoid class drug (optionally ciclesonide) is by inhalation (i.e., they are inhaled);
a hydrocortisone or cortisol (optionally CORTEF™, SOLUCORTEF™), optionally hydrocortisone sodium succinate or hydrocortisone acetate or dexamethasome (optionally Dextenza™ Ozurdex™ Neofordex™),
and optionally the corticosteroid or glucocorticoid class drug (for example budesonide or ciclesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,
and optionally the corticosteroid or glucocorticoid class drug (optionally budesonide or ciclesonide) is formulated as a powder or for administration in an inhaler or by nasal spray, or for rectal administration,
and optionally the corticosteroid or glucocorticoid class drug (for example, budesonide or ciclesonide) is administered together with or in combination with 10 mg to 80 mg, an antibiotic (optionally azithromycin or a tetracycline class drug,
wherein optionally the tetracycline class drug comprises doxycycline, or DORYX™, DOXYHEXA™, DOXYLIN™), zinc, zinc salt or zinc chelate and/or a vitamin (optionally vitamin D or calcifediol, D2 (or ergocalciferol), D3 (or cholecalciferol), C, E, B12, B6);
an anti-androgen drug, and optionally the anti-androgen drug is bicalutamide, optionally CASODEX™, or dutasteride (or AVODART™),
and optionally the anti-androgen drug is a nonsteroidal anti-androgen (NSAA) or an androgen receptor (AR) antagonist, and optionally the NSAA or AR antagonist comprises proxalutamide (or its developmental name GT-0918) (Suzhou Kintor Pharmaceuticals, Inc., a subsidiary of Kintor Pharmaceutical Limited), or flutamide (or niftolide, or EULEXIN™), or bicalutamide (or CASODEX™) or enzalutamide (or XTANDI™),
and optionally the anti-androgen drug comprises a 5α-reductase inhibitor, and optionally the 5α-reductase inhibitor comprises finasteride (or PROSCAR™, PROPECIA™, or FINIDE™),
and optionally the anti-androgen drug, or NSAA, or proxalutamide or bicalutamide, is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;
an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2),
and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin or a tetracycline class drug, wherein optionally the tetracycline class drug comprises doxycycline, or DORYX™, DOXYHEXA™, DOXYLIN™); chloroquine (or ARALEN™), chloroquine phosphate, chloroquine diphosphate and/or hydroxychloroquine (optionally, PLAQUENIL™); zinc, zinc salt or zinc chelate; remdesivir (optionally, GS-5734™, Gilead Sciences); oseltamivir (or TAMIFLU™); and/or, hydrocortisone; or, any combination thereof;
a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or a tetracycline tetracycline class drug, and optionally the tetracycline class drug comprises doxycycline, or DORYX™, DOXYHEXA™, DOXYLIN™;
a dendrimer, optionally astodrimer sodium (Starpharma, Melbourne, Australia);
an antihistamine class drug such as azelastine, or ASTELIN™ OPTIVAR™, ALLERGODIL™, brompheniramine, fexofenadine or ALLEGRA™′ pheniramine or AVIL™ or chlorpheniramine;
a selective serotonin reuptake inhibitor (SSRI) class drug, optionally fluvoxamine, or LUVOX™ FAVERIN™ FLUVOXIN™;
a nicotinic antagonist, a dopamine agonist or a noncompetitive N-Methyl-d-aspartic acid or N-Methyl-d-aspartate (NMDA) antagonist, wherein optionally the nicotinic antagonist, dopamine agonist or noncompetitive NMDA antagonist is 1-adamantylamine or amantadine, or GOCOVRI™, SYMADINE™, SYMMETREL™, optionally administered or dosaged at between about 50 mg to 150 mg, or about 100 mg, or 200 mg, per day for a period of between about 7 and 21 days, or about 14 days, and optionally the nicotinic antagonist, dopamine agonist or noncompetitive NMDA antagonist is also administered or formulated with an antibiotic (optionally azithromycin or doxycycline), ivermectin, hydroxychloroquine (optionally, PLAQUENIL™) and/or zinc, zinc salt or zinc chelate (optionally zinc sulfate, optionally at (50 mg daily), and optionally the amantadine is formulated or administered at 100 mg per day for the first two days of treatment, which optionally can then be elevated to 100 mg twice daily, optionally for the next 10 days;
an acetaldehyde dehydrogenase inhibitor, optionally disulfiram, or ANTABUS™, or ANTABUSE™, optionally formulated as an extended, sustained or slow-release disulfiram formulation, optionally the extended, sustained or slow-release disulfiram is formulated as a tablet, a capsule or in an injectable, amphiphilic, absorbable, depot-forming drug delivery system (DDS),
and optionally the DDS system comprises: a polyether ester urethane comprising 65% d,l-lactide, 19% polyethylene glycol, and 16% glycolide interlinked with an aliphatic di-isocyanate, or comprises VISCOPRENE™,
and optionally the acetaldehyde dehydrogenase inhibitor, optionally disulfiram, is formulated as an injectable formulation, optionally formulated in saline, optionally formulated as a slurry in saline as described in U.S. Pat. No. 4,678,809A, optionally formulated at about one gram (g) for a bolus injection, optionally subcutaneously;
an immunosuppressive drug, wherein optionally the immunosuppressive drug comprises tocilizumab or atlizumab, or Actemra™, or RoActemra™, or a calcineurin inhibitor (CNI), wherein the CNI comprises ciclosporin (or cyclosporine or cyclosporin), or Neoral™, or Sandimmune™, or tacrolimus, or Protopic™, or Prograf™, and optionally the immunosuppressive drug is also administered or formulated with an antibiotic (optionally azithromycin or doxycycline), ivermectin, hydroxychloroquine (optionally, PLAQUENIL™) and/or zinc, zinc salt or zinc chelate (optionally zinc sulfate, optionally at (50 mg daily),
and optionally the calcineurin inhibitor (CNI), wherein the CNI comprises ciclosporin (or cyclosporine or cyclosporin) is formulated combination of CNI (optionally cyclosporine) at a dose of 3 mg/kg (180 mg daily) together with 12 mg ivermectin once, and optionally also plus zinc 50 mg base and doxycycline 100 mg bid, optionally all for 10 days;
a protein kinase inhibitor, wherein optionally the protein kinase inhibitor is a p38 mitogen-activated protein kinase inhibitor, or ralimetinib, and optionally the protein kinase inhibitor is also administered or formulated with an antibiotic (optionally azithromycin or doxycycline), ivermectin, hydroxychloroquine (optionally, PLAQUENIL™) and/or zinc or any zinc salt or zinc chelate (optionally zinc sulfate, optionally at (50 mg daily);
an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally meplazumab), a cyclooxygenase (COX) (optionally, COX2) inhibitor, a glucocorticoid (optionally a synthetic glucocorticoid, hydrocortisone, dexamethasone (or DEXTENZA™, OZURDEX™, or NEOFORDEX™) or cortisol, or CORTEF™), plitidepsin or dehydrodidemnin B, or APLIDIN™, or a nonsteroidal anti-inflammatory drug (NSAID), wherein optionally the NSAID comprises indomethacin (or indomethacin) or INDOCID™ or INDOCIN™ or naproxen, or NAPROSYN™ or ALEVE™ or a cyclooxygenase inhibitor, or a COX-1 or an COX-2 inhibitor, or aspirin, or ibuprofen or ADVIL™′ MOTRIN™ or NUROFEN™ or celecoxib or CELEBREX™, or parecoxib or DYNASTAT™, or etoricoxib or ARCOXIA™,
and optionally the anti-inflammatory therapy or anti-inflammatory therapy drug is also administered or formulated with an antibiotic (optionally azithromycin or doxycycline), ivermectin, hydroxychloroquine (optionally, PLAQUENIL™) and/or zinc or any zinc salt or zinc chelate (optionally zinc sulfate, optionally at (50 mg daily),
and optionally opaganib, or YELIVA™, or opaganib, or YELIVA™ administered or formulated together with an oral and/or inhaled or aerosol chloroquine (or ARALEN™), chloroquine phosphate, chloroquine diphosphate and/or hydroxychloroquine (optionally, PLAQUENIL™),
and optionally the opaganib or YELIVA™ is formulated or administered at a dosage of QD (once a day), bid (twice a day) or tid (three times a day) at a dosage of between about 100 to 600 mg per day or per dosage, or at about 100, 200, 300, 400, 500 or 600 mg per day or per dosage,
and optionally the opaganib, or YELIVA™ is also administered or formulated with an antibiotic (optionally azithromycin or doxycycline), ivermectin (optionally at 12 mg ivermectin, optionally administered on days 1, 3, 6 and 8), hydroxychloroquine (optionally, PLAQUENIL™) and/or zinc or any zinc salt or zinc chelate (optionally zinc sulfate, optionally at (50 mg daily);
a calcium channel blocker, or verapamil (or ISOPTIN™ CALAN™), or a voltage gated potassium (KCNH2) channel or a voltage gated calcium channel (CACNA2D2) blocker, or amiodarone (or CORDARONE™ NEXTERONE™);
a suramin, or ANTRYPOL™ BAYER 305™, or GERMANIN™;
a PPAR agonist, optionally fenofibrate, or TRICOR™, FENOBRAT™, FENOGLIDE™ or LIPOFEN™ and optionally the PPAR agonist comprises bezafibrate, or BEZALIP™ or combination of bezafibrate and chenodeoxycholic acid, or HEPACONDA™, or aluminium clofibrate, or alfibrate, or ciprofibrate, or clinofibrate or LIPOCLIN™ or clofibrate or ATROMID-S™ or clofibride, or gemfibrozil or LOPID™, or ronifibrate, or simfibrate or CHOLESOLVIN™, or any combination thereof, or a combination of fenofibrate and simvastatin, or CHOLIB™;
a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™, Glenmark Pharmaceuticals),
wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 2 to 15 days, or for about 2, 3, 4, 5, 6, 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days,
and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin),
and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin) with an antibiotic, and optionally the antibiotic comprises azithromycin, minocycline, amoxicillin, niclosamide, nitazoxanide, hydroxychloroquine or doxycycline), and optionally the synthetic nucleoside analog or derivative, avermectin class drug, and antibiotic are administered together or as separate formulations, and optionally are administered every one, two, three, four or five weeks for between about one month and one year or more;
and optionally molnuvpiravir, ivermectin and hydroxychloroquine are administered together or as separate formulations, and optionally are administered every one, two, three, four or five weeks for between about one month and one year or more;
and optionally the synthetic nucleoside analog or derivative (optionally N4-hydroxycytidine, or the prodrug of N4-hydroxycytidine, optionally molnuvpiravir or favipiravir), and antibiotic (optionally doxycycline or hydroxychloroquine) is administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D),
and optionally the synthetic nucleoside analog or derivative, or N4-hydroxycytidine, or the prodrug of N4-hydroxycytidine, optionally molnuvpiravir or favipiravir, is administered with an antibiotic (optionally the antibiotic comprises azithromycin, minocycline, amoxicillin, niclosamide, nitazoxanide, hydroxychloroquine or doxycycline), optionally also administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
and optionally any of these combinations is administered very 2, 3, 4, 5, 6, 7, 8, 9 or 10 or more days for between about 1 month and one year or more;
an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2);
at least one vitamin, wherein optionally the at least one vitamin comprises: vitamin B3 (or pyridine-3-carboxylic acid, niacin or nicotinic acid, or vitamin B3 or niacin administered as a slow release form (or NIASPAN FCT™), vitamin D (optionally D2, or ergocalciferol), or Vitamin D3 or cholecalciferol, optionally administered at about 1000 to 4000 ugm/day; vitamin B12, vitamin B6 (or pyridoxine); vitamin K; vitamin A; vitamin E; and/or, vitamin C (optionally administered at 500 mg bid);
copper, optionally administered or formulated at a dosage of between about 1 to 200 mg per day, wherein optionally the copper is administered or formulated as cupric chloride and administered intravenously formulated at about 0.4 mg/ml;
selenium, optionally administered as selenious acid formulated at about 65.4 mcg/ml (or pimp, and optionally the selenium is administered at a dosage of between about 50 to 100 p/ml, optionally between about 60 to 100 pgm per day is administered to an adult, and only up to 60 pgm per day for pediatric patients;
favipiravir (or T-705, avigan, or favilavir), optionally at 800 mg bid;
zinc, zinc salt or zinc chelate (optionally a zinc sulphate, acetate, gluconate or picolinate) or zinc oxide nanoparticles, optionally at a dosage of between about 1 mg to 250 mg;
colchicine, or COLCRYS™, MITIGARE™;
at least one antibiotic or anti-viral (wherein optionally the antibiotic is doxycycline (optionally, DORYX™, DOXYHEXA™, DOXYLIN™) (optionally formulated or administered at a dosage of between about 25 mg to 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
at least one anti-viral drug or medication, or anti-microbial drug, or palliative agent or drug, wherein optionally the anti-viral drug or medication, or anti-microbial drug, is or comprises efavirenz (for example, SUSTIVA™), tenofovir (optionally tenofovir alafenamide or tenofovir disoproxil, or VIREAD™), emtricitabine and tenofovir, nevirapine (or the combination efavirenz with emtricitabine and tenofovir, or ATRIPLA™), amprenavir (for example, AGENERASE™), nelfinavir (for example, VIRACEPT™) and/or remdesivir (for example, GS-5734™, Gilead Sciences), a viral RNA-dependent RNA polymerase inhibitor, optionally favipiravir (optionally AVIGAN™) or sofosbuvir (optionally SOVALDI™, SOFORAL™); or, an adenosine analog (optionally galidesivir, optionally BCX4430, IMMUCILLIN-A™),
and optionally the anti-viral drug or medication is or comprises an anti-retroviral drug or drug combination, and optionally the anti-retroviral drug or drug combination comprises: darunavir and cobicistat (for example, REZOLSTA™ or PREZCOBIX™); atazanavir (or REYATAZ™) and cobicistat (or EVOTAZ™); abacavir, lamivudine and dolutegravir (TRIUMEQ™); tenofovir (or tenofovir disoproxil or tenofovir disoproxil, or VIREAD™, or emtricitabine) and elvitegravir and cobicistat (for example, STRIBILD™); tenofovir (or disoproxil or emtricitabine) and elvitegravir and cobicistat (COMPLERA™ or EVIPLERA™); efavirenz (optionally, SUSTIVA™), emtricitabine and tenofovir (or ATRIPLA™); lamivudine, nevirapine and stavudine (for example, TRIOMUNE™); atazanavir (or REYATAZ™) and cobicistat (for example, EVOTAZ™); lamivudine and raltegravir (for example, DUTREBIS™); lamivudine and dolutegravir (or DOVATO™); doravirine, lamivudine and tenofovir (for example, DELSTRIGO™); or lamivudine, zidovudine and nevirapine (for example, CUOVIR-NTH), and optionally the anti-viral drug or drug combination comprises daclatasvir (optionally DAKLINZA™);
a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day), and optionally also including colchicine;
a viral, or a coronavirus or a COVID-19, protease inhibitor, wherein optionally the protease inhibitor comprises: ASC09 (CAS registry no. 1000287-05-7) (Janssen Research and Development, LLC), ritonavir (optionally NORVIR™) or ASC09 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir, cobicistat or darunavir and cobicistat, or PF-07321332, or nirmatrelvir, or the combination of nirmatrelvir and ritonavir, or PAXLOVID™, PF-07304814 or PF-008335231 (Pfizer), or remdesivir (for example, GS-5734™, Gilead Sciences) or ritonavir (optionally NORVIR™) in combination with the PF-07321332, or nirmatrelvir, or the combination of nirmatrelvir and ritonavir, or PAXLOVID™, PF-07304814 or PF-008335231 (Pfizer) optionally as an oral formulation, optionally as a tablet, geltab or capsule, 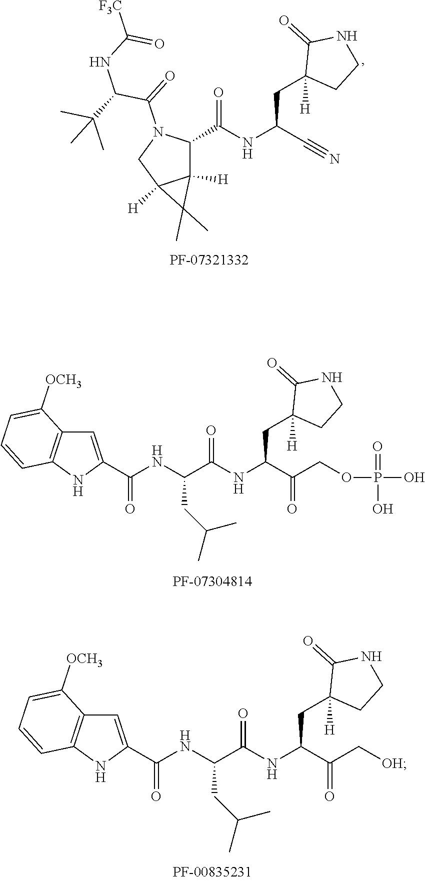 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C ; COc1cccc2[nH]c(C(=O)N[C@@H](CC(C)C)C(=O)N[C@@H](C[C@@H]3CCNC3=O)C(=O)COP(=O)(O)O)cc12 ; COc1cccc2[nH]c(C(=O)N[C@@H](CC(C)C)C(=O)N[C@@H](C[C@@H]3CCNC3=O)C(=O)CO)cc12]
a thiazolide class drug, optionally nitazoxanide (or ALINIA™′ NIZONIDE™) or tizoxanide (or 2-Hydroxy-N-(5-nitro-2-thiazolyl)benzamide);
a blood clot inhibiting drug such as aspirin, warfarin (or COUMADIN™) or rivaroxaban (or XARELTO™);
lopinavir, ritonavir (optionally NORVIR™) or the combination of lopinavir and ritonavir (or KALETRA™, ALTERA™, ALUVIA™, KALMELTREX, or LOPIMUNE™), opaganib (or YELIVA™), oseltamivir (or TAMIFLU™), and/or zanamivir (or RELENZA™);
an inhibitor or S-phase kinase-associated protein 2 (SKP2), or dioscin, or niclosamide, or NICLOCIDE™, FENASAL™, or PHENASAL™;
a tyrosine kinase inhibitor (TKi), wherein the TKi comprises: masitinib (or MASIVET™, or KINAVET™); or imatinib (or GLEEVEC™, GLIVEC™); or gefitinib (or IRESSA™), or erlotinib (or TARCEVA™), or dasatinib (or SPRYCEL™, DASANIX™);
ribavirin or tribavirin (or COPEGUS™, REBETOL™, or VIRAZOLE™), interferon beta 1 b, or a combination of ribavirin and interferon beta, or a combination of lopinavir and ritonavir (optionally NORVIR™) and interferon-beta-1b;
a nucleoside analog reverse-transcriptase inhibitor (NRTI) (optionally abacavir, or ZIAGEN™), acyclovir (or ZOVIRAX™), optionally ACICLOVIR™), adefovir (optionally HEPSERA™), amantadine (optionally GOCOVRI™, SYMADINE™, SYMMETREL™), rintatolimod (or AMPLIGEN™), amprenavir (optionally, AGENERASE™), aprepitant (or EMEND™), umifenovir (or ARBIDOL™), atazanavir (or REYATAZ™), tenofovir or tenofovir disoproxil (or VIREAD™), a combination of efavirenz and emtricitabine and tenofovir (or ATRIPLA™), balavir, baloxavir marboxil (XOFLUZA™), bepotastine (or TALION™, BEPREVE™), bevirimat, bictegravir, biktarvy, brilacidin, cidofovir, caspofungin, lamivudine and zidovudine (optionally, COMBVIR™), cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir (or VIRACEPT™), nevirapine, nexavir, nitazoxanide, norvir, a nucleoside analogue (optionally brincidofovir, didanosine, favipiravir (also known as T-705, AVIGAN™, or favilavir, Toyama Chemical, Fujifilm, Japan), vidarabine, galidesivir (optionally, BCX4430, IMMUCILLIN-A™), remdesivir (optionally, GS-5734™, Gilead Sciences), cytarabine, gemcitabine, emtricitabine, lamivudine, zalcitabine, entecavir, stavudine, telbivudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyrimidine, raltegravir, rifampicin, ribavirin or tribavirin (or COPEGUS™, REBETOL™, or VIRAZOLE™), rilpivirine, rimantadine, ritonavir (optionally NORVIR™), saquinavir, sofosbuvir, stavudine, telaprevir, tegobuv, tenofovir (optionally tenofovir alafenamide, tenofovir disoproxil or VIREAD™), tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™) or any combination thereof;
fenofibrate, or TRICOR™, FENOBRAT™, FENOGLIDE™, or LIPOFEN™, or a combination of fenofibrate and simvastatin, or CHOLIB™;
suramin, or ANTRYPOL™, BAYER 305™, or GERMANIN™;
a synthetic nucleoside analog or derivative, or N4-hydroxycytidine, or a prodrug of N4-hydroxycytidine, optionally molnuvpiravir (Merck), or favipiravir (also known as T-705 or AVIGAN™, or favilavir, Toyama Chemical, Fujifilm, Japan, or FABIFLU™ Glenmark Pharmaceuticals),
wherein the synthetic nucleoside analog or derivative, or N4-hydroxycytidine, or the prodrug of N4-hydroxycytidine, optionally molnuvpiravir or favipiravir, is given as between about 10 mg to 3 gm per dose, or between about 10 mg to 3 gm per day, or can be dosed either as a single dose or given one, two, three or four times a day, or is administered at 200 to 800 mg twice daily, or 200, 400, 600 or 800 mg twice daily, or at 200 to 800 mg three times a day, or at 200, 400, 600 or 800 mg three times a day, or is administered at 200 to 800 mg three times a day for between about 5 to 15 days, or for about 7, 8, 9, 10, 11 or 12 days, and optionally when combined with other drugs a lower dosage is used, optionally administered at 100 or 200 mg three times a day for between about 5 to 15 days, or for about 7, 8, 9, 10, 11 or 12 days,
and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin),
and optionally the synthetic nucleoside analog or derivative, or N4-hydroxycytidine, or the prodrug of N4-hydroxycytidine, optionally molnuvpiravir or favipiravir, is administered with an avermectin class drug (optionally ivermectin) and an antibiotic, and optionally the antibiotic comprises azithromycin, minocycline, amoxicillin, niclosamide, nitazoxanide, hydroxychloroquine or doxycycline), and optionally the synthetic nucleoside analog or derivative, avermectin class drug, and antibiotic are administered together or as separate formulations, and optionally are administered every one, two, three, four or five weeks for between about one month and one year or more;
and optionally molnuvpiravir, ivermectin and hydroxychloroquine are administered together or as separate formulations, and optionally are administered every one, two, three, four or five weeks for between about one month and one year or more;
and optionally the synthetic nucleoside analog or derivative (optionally N4-hydroxycytidine, or the prodrug of N4-hydroxycytidine, optionally molnuvpiravir or favipiravir), and antibiotic (optionally doxycycline or hydroxychloroquine) is administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D),
and optionally the synthetic nucleoside analog or derivative, or N4-hydroxycytidine, or the prodrug of N4-hydroxycytidine, optionally molnuvpiravir or favipiravir, is administered with at least two antibiotics (optionally the at least two antibiotics comprise azithromycin, minocycline, amoxicillin, niclosamide, nitazoxanide, hydroxychloroquine and/or doxycycline), and a zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
an antibody or antibody or vaccine therapy for treating, preventing or ameliorating a microbial or a viral infection (optionally a coronavirus infection, optionally a COVID-19 infection) or a microbial infection (optionally a protozoan, helminthiasis, insect and/or parasitic infection), and optionally the antibody comprises a monoclonal antibody, and optionally the monoclonal antibody comprises sotrovimab (GlaxoSmithKline and Vir Biotechnology), or casirivimab, imdevimab or casirivimab and imdevimab (REGEN-COV™) (Regeneron), or bamlanivimab oretesevimab or bamlanivimab and etesevimab (Junshi Biosciences), or tocilizumab or ACTEMRA™ or ROACTEMRA™ (Hoffmann-La Roche),
and optionally the antibody or vaccine therapy comprises tozinameran or COMIRNATY™ (Pfizer), or elasomeran or SPIKEVAX™ (Moderna), or SPUTNIK V™ or Gam-COVID-Vac (Gamaleya Research Institute), or AZD1222 or COVISHIELD™ or VAXZEVRIA™ (Oxford-AstraZeneca),
and optionally the antibody or antibody therapy comprises or is contained in a convalescent sera or plasma,
wherein optionally any of these combinations is administered very 2, 3, 4, 5, 6, 7, 8, 9 or 10 or more days for between about 1 month and one year or more; and/or
or any combination thereof.

14. The method of claim 1, wherein a combination of an avermectin class drug (optionally ivermectin) (optionally dosaged at between about 30 to 80 mg per day, or between about 36 to 60 mg per day), clofazimine (optionally dosaged at about 100 mg or 150 mg per day, or between about 50 mg and 200 mg per day), doxycycline or azithromycin (optionally dosaged at about 100 mg or 150 mg per day, or between about 50 mg and 200 mg per day) and zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) are administered:
(a) once a month; or
(b) for the first four, five, six or seven days of treatment an avermectin class drug (optionally ivermectin) is given at about 24 mg per day or between about 20 to 30 mg per day, doxycycline or azithromycin is given at about 100 mg per day or between about 50 and 150 mg per day, clofazimine is given about 100 mg per day or between about 50 and 150 mg per day, and zinc, zinc salt or zinc chelate (optionally a zinc sulphate, acetate, gluconate or picolinate, or zinc oxide nanoparticles) is administered at a dosage of between about 25 mg to 100 mg per day, or about 50 mg per day),
and after this initial first four, five, six or seven days of treatment a once a month maintenance regimen of an avermectin class drug (optionally ivermectin) dosaged at between about 60 to 80 mg, or about 60 mg, clofazimine dosaged at about 100 mg or between about 50 to 150 mg, doxycycline or azithromycin dosaged at about 100 mg or between about 50 to 150 mg, and zinc, zinc salt or zinc chelate (optionally a zinc sulphate, acetate, gluconate or picolinate, or zinc oxide nanoparticles) is administered at a dosage of between about 25 mg to 100 mg per day, or about 50 mg per day) is given,
and optionally the avermectin class drug (optionally ivermectin), clofazimine, doxycycline or azithromycin and zinc are formulated and administered in separate dosage units (optionally geltabs, tablets, capsules), or the avermectin class drug (optionally ivermectin), clofazimine, doxycycline or azithromycin and zinc, zinc salt or zinc chelate are formulated and administered in one unit dosage (optionally all in one a geltab, tablet, capsule).

15. The method of claim 1, wherein the individual in need thereof suffers from long term effects, or chronic effects or symptoms, of a viral infection, or the individual in need thereof has not fully recovered from the viral infection weeks or even months after first experiencing symptoms, or the individual in need thereof experiences continuous symptoms for weeks or months after being first diagnosed or treated with the viral infection, or the individual in need thereof feels better for weeks, then relapses with old or new symptoms,
and optionally the medication or the drug combination is administered to prevent a so-called “long-hauler” syndrome, or to treat or prevent continuous symptoms for weeks or months, or to prevent or treat relapsing with old or new symptoms,
wherein optionally the viral infection is a COVID-19 infection.

16. The method of claim 1, wherein the synthetic nucleoside analog or derivative, or N4-hydroxycytidine, or the prodrug of N4-hydroxycytidine, optionally molnuvpiravir or favipiravir, is administered with an antibiotic (optionally the antibiotic comprises azithromycin, minocycline, amoxicillin, niclosamide, nitazoxanide, hydroxychloroquine or doxycycline), optionally also administered with zinc, zinc salt or zinc chelate (optionally a zinc sulphate, acetate, gluconate or picolinate, or zinc oxide nanoparticles, optionally at a dosage of between about 1 mg to 250 mg, or about 50 mg per day) and/or a vitamin, optionally vitamin C or D,
and optionally the synthetic nucleoside analog or derivative, or N4-hydroxycytidine, or the prodrug of N4-hydroxycytidine, optionally molnuvpiravir or favipiravir, is administered with azithromycin and/or hydroxychloroquine and/or doxycycline with an avermectin class drug optionally comprising: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
and optionally the synthetic nucleoside analog or derivative, or N4-hydroxycytidine, or the prodrug of N4-hydroxycytidine, optionally molnuvpiravir or favipiravir, is administered with opinavir, ritonavir (optionally NORVIR™), or the combination lopinavir and ritonavir (or KALETRA™, ALTERA™, ALUVIA™, KALMELTREX or LOPIMUNE™), opaganib (or YELIVA™), oseltamivir (or TAMIFLU™), and/or zanamivir (or RELENZA™).

17. A product of manufacture comprising a drug or drug combination (or therapeutic drug combination) as set forth in claim 1, wherein optionally the product of manufacture comprise or is manufactured or fabricated as a kit, blister pack or package, a clamshell, a tray, a shrink wrap, or equivalent,
and optionally the product of manufacture contains or has fabricated therein a first delivery packet (or, what is indicated or configured on the package, kit or container comprises a blister package, a clamshell, a tray, a shrink wrap and the like to be the first dosage taken by the individual) comprising: dosage of an avermectin class drug (optionally ivermectin); or, a loading dosage of an avermectin class drug (optionally ivermectin),
and optionally the avermectin class drug is dosaged at about 30 mg/kg (or 30 mg per 2.2 pounds (lb)) or about 1200 mg or 1600 mg to about 1800 mg in a 60 kg (about 132 lb) person,
and optionally the loading dosage of the avermectin class drug is between about 30 to 60 mg/kg or is between about 1600 mg to 1800 mg to 3600 mg in a 60 kg (about 132 lb) person,
and optionally the product of manufacture contains or has fabricated therein one or more additional delivery packets containing therein an additional drug or drugs, or vitamin or nutritional supplement.

18-19. (canceled)
